SEQUENCE LISTING (11 sequences)

SEQ ID NO: 1 - protein, 253 aa, synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSASTGGGGSGGGGSGGGGSVHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 2 - protein, 125 aa, synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSAST

SEQ ID NO: 3 - protein, 9 aa, synthetic construct
ITFDDAVMH

SEQ ID NO: 4 - protein, 6 aa, synthetic construct
GISSNS

SEQ ID NO: 5 - protein, 15 aa, synthetic construct
VKGLYSNPRGGAFDI

SEQ ID NO: 6 - protein, 113 aa, synthetic construct
VHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 7 - protein, 11 aa, synthetic construct
GGNNIGTKSVH

SEQ ID NO: 8 - protein, 7 aa, synthetic construct
ADSDRPS

SEQ ID NO: 9 - protein, 12 aa, synthetic construct
QVWDSRSDHLWV

SEQ ID NO: 10 - protein, 510 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNAS

SEQ ID NO: 11 - protein, 788 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNASGGGGSGGGGSGGGGSGGGGSGGGGSEVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSASTGGGGSGGGGSGGGGSVHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

(19) United States
(12) Patent Application Publication
(10) Pub. No.: US 2025/0326811 A1
(43) Pub. Date: Oct. 23, 2025

(54) IL-12-ALBUMIN-BINDING DOMAIN FUSION PROTEIN FORMULATIONS AND METHODS OF USE THEREOF

(71) Applicant: SONNET BIOTHERAPEUTICS, INC., Princeton, NJ (US)

(72) Inventors: John K. CINI, Blairstown, NJ (US); Indu JAVERI, Wilmington, MA (US)

(21) Appl. No.: 18/869,328

(22) PCT Filed: May 26, 2023
(86) PCT No.: PCT/US2023/067566

§ 371 Date: Nov. 26, 2024

Related U.S. Application Data
(60) Provisional application No. 63/346,368, filed on May 27, 2022.

(51) Int. Cl. C07K 14/54; A61K 9/08; A61K 9/19; A61K 38/00; A61K 47/18; A61K 47/26

(52) U.S. Cl. CPC C07K 14/5434; A61K 9/08; A61K 9/19; A61K 47/183; A61K 47/26; A61K 38/00; C07K 2319/31

(57) ABSTRACT

The present disclosure provides a formulation of an IL-12-Albumin Binding Domain (ABD) fusion protein, comprising about 10 μg/mL to about 50 μg/mL fusion protein, about 25 mM to about 100 mM alanine, about 100 mM to about 400 mM trehalose, about 10 mM to about 50 mM glycylglycine, about 0.01% to about 0.04% polysorbate 20 (v/v), and about 5 μM to about 20 μM diethylenetriaminepentaacetic acid (DTPA), wherein the formulation has a pH of about 6.8 to about 8.0. Such formulations may be useful to treat and/or prevent IL-12 related diseases or disorders in a subject in need thereof.

### CROSS-REFERENCE TO RELATED APPLICATIONS

This application claims priority to U.S. Provisional Patent Application No. 63/346,368, filed on May 27, 2023, which is hereby incorporated by reference in its entirety.

### FIELD

The present disclosure generally provides IL-12-Albumin-Binding Domain (ABD) fusion protein formulations and method of use thereof including for the treatment of IL-12-related diseases and/or disorders.

### BACKGROUND

IL-12 is capable of mediating immune effector functions in a manner compatible to enhancing pro-inflammatory, endogenous anti-tumor immune response. (See, e.g., Boggio et al., J Exp Med 188:589-96 (1998); Cavallo et al., Cancer Res 59:414-21 (1999); Yu et al. Int Immunol 8:855-65 (1996); Nastala et al., J Immunol 153:1697:706 (1994); Brunda et al., J Exp Med 178:1223-30 (1993). IL-12 is known to induce an inflammatory Th1 CD4+ T cell response as well as enhance CD8+ T cell cytotoxicity. Studies have also shown that T cell secretion of IFNγ mediated by IL-12 may reverse T cell anergy and confer effector T cell resistance to immune suppressive regulatory T cells. The ability of IL-12 to not only activate the adaptive and the innate immune systems, but also further modulate the otherwise immune-hostile tumor microenvironment makes IL-12 an ideal candidate for tumor immunotherapy.

Serum albumin possesses a long half-life in the range of 2-4 weeks due to recycling through the neonatal Fc receptor (FcRn). Albumin is taken up by endothelial cells through macropinocytosis and binds to the FcRn in a pH-dependent manner in the acidic environment of the early endosome. Albumin-FcRn binding diverts albumin molecules from degradation in the lysosomal compartment and redirects the albumin molecules to the plasma membrane, where they are released back into the blood plasma due to the neutral pH.

Albumin binding domain (ABD) fusion proteins as described in U.S. Pat. No. 11,028,166 are useful for extending the half-lives of biologics (e.g., interleukins and antibodies). Such ABDs do not compete with FcRN for albumin binding and bind albumin at a pH range that allows for the ABD to also undergo FcRn-driven endosomal albumin recycling when bound to albumin. As such, biologics that include the ABD are capable of evading lysosomal degradation using the albumin-FcRn pathway and, consequently, exhibit longer serum half-lives than counterparts lacking ABDs. Moreover, such ABD containing therapeutics advantageously localize to tumors, which are known to contain high levels of serum albumin. Thus, such ABD containing therapeutics are particularly useful for the treatment of cancers.

The therapeutic use of ABD fusion proteins is facilitated by formulations that retain stability of the fusion protein under a variety of conditions. It is important that the therapeutic formulation permits storage without an unacceptable loss of activity of the active protein, minimizes the accumulation of undesirable products such as aggregates or degraded species (e.g., fragmented, oxidized, deamidated or isomerized species), accommodates appropriate concentrations of the protein, and does not contain components that are incompatible with therapeutic applications. Thus, there exists a need in the art for a stable pharmaceutical formulation comprising an IL-12-ABD fusion protein, which is suitable for therapeutic use.

### SUMMARY

The present disclosure provides IL12-ABD fusion protein formulations that are uniquely stable in both liquid and solid (e.g., lyophilized) forms at low concentrations of the fusion protein. Such stable liquid pharmaceutical formulations comprise about 10 μg/mL to about 50 μg/mL IL12-ABD fusion protein of SEQ ID NO: 11, about 25 mM to about 100 mM alanine, about 100 mM to about 400 mM trehalose, about 10 mM to about 50 mM glycylglycine, about 0.01% to about 0.04% polysorbate 20 (v/v); and about 5 μM to about 20 μM diethylenetriaminepentaacetic acid (DTPA), wherein the formulation has a pH of about 6.8 to about 8.0.

In some embodiments, the liquid formulation comprises the fusion protein of SEQ ID NO: 11 in an amount of about 10 μg/mL to about 30 μg/mL, about 15 μg/mL to about 25 μg/mL, about 10 μg/mL to about 15 μg/mL, about 15 μg/mL to about 20 μg/mL, about 20 μg/mL to about 25 μg/mL, about 25 μg/mL to about 30 μg/mL, about 30 μg/mL to about 35 μg/mL, about 35 μg/mL to about 40 μg/mL, about 40 μg/mL to about 45 μg/mL, about 45 μg/mL to about 50 μg/mL, about 10 μg/mL, about 15 μg/mL, about 20 μg/mL, about 25 μg/mL, about 30 μg/mL, about 35 μg/mL, about 40 μg/mL, about 45 μg/mL, and/or about 50 μg/mL.

In some embodiments, the liquid formulation comprises the fusion protein of SEQ ID NO: 11 in an amount of about 10 μg/mL to about 30 μg/mL.

In some embodiments, the liquid formulation comprises about 20 μg/mL fusion protein of SEQ ID NO: 11.

In some embodiments, the liquid formulation comprises about 25 mM to about 75 mM alanine, about 40 mM to about 60 mM alanine, about 25 mM to about 30 mM alanine, about 30 mM to about 40 mM alanine, about 40 mM to about 50 mM alanine, about 50 mM to about 60 mM alanine, about 60 mM to about 70 mM alanine, about 70 mM to about 80 mM alanine, about 80 mM to about 90 mM alanine, about 90 mM to about 100 mM alanine, about 25 mM alanine, about 30 mM alanine, about 40 mM alanine, about 50 mM alanine, about 60 mM alanine, about 70 mM alanine, about 80 mM alanine, about 90 mM alanine, and/or about 100 mM alanine.

In some embodiments, the liquid formulation comprises about 50 mM alanine.

In some embodiments, the liquid formulation comprises about 100 mM to about 300 mM trehalose, about 150 mM to about 250 mM trehalose, about 100 mM to about 150 mM trehalose, about 150 mM to about 200 mM trehalose, about 200 mM to about 250 mM trehalose, about 250 mM to about 300 mM trehalose, about 300 mM to about 350 mM trehalose, about 350 mM to about 400 mM trehalose, about 100 mM trehalose, about 150 mM trehalose, about 200 mM trehalose, about 250 mM trehalose, about 300 mM trehalose, about 350 mM trehalose, and/or about 400 mM trehalose.

In some embodiments, the liquid formulation comprises about 200 mM trehalose.

In some embodiments, the liquid formulation comprises about 10 mM to about 40 mM glycylglycine, about 10 mM to about 30 mM glycylglycine, about 10 mM to about 15 mM glycylglycine, about 15 mM to about 20 mM glycylglycine, about 20 mM to about 25 mM glycylglycine, about 25 mM to about 30 mM glycylglycine, about 35 mM to about 40 mM glycylglycine, about 40 mM to about 45 mM glycylglycine, about 45 mM to about 50 mM glycylglycine, about 10 mM glycylglycine, about 15 mM glycylglycine, about 20 mM glycylglycine, about 25 mM glycylglycine, about 30 mM glycylglycine, about 35 mM glycylglycine, about 40 mM glycylglycine, about 45 mM glycylglycine, and/or about 50 mM glycylglycine.

In some embodiments, the liquid formulation comprises about 25 mM glycylglycine.

In some embodiments, the liquid formulation comprises about 0.01% to about 0.03% polysorbate 20 (v/v), about 0.01% to about 0.02% polysorbate 20 (v/v), about 0.02% to about 0.03% polysorbate 20 (v/v), about 0.03% to about 0.04% polysorbate 20 (v/v), about 0.01% polysorbate 20 (v/v), about 0.02% polysorbate 20 (v/v), about 0.03% polysorbate 20 (v/v), and/or about 0.04% polysorbate 20 (v/v).

In some embodiments, the liquid formulation comprises about 0.02% polysorbate 20 (v/v).

In some embodiments, the liquid formulation comprises about 5 μM to about 15 μM DTPA, about 5 μM to about 10 μM DTPA, about 10 μM to about 15 μM DTPA, about 15 μM to about 20 μM DTPA, about 5 μM DTPA, about 10 μM DTPA, about 15 μM DTPA, and/or about 20 μM DTPA.

In some embodiments, the liquid formulation comprises about 10 μM DTPA.

In some embodiments, the liquid formulation has a pH of about 7.0 to about 7.8, about 7.2 to about 7.8, about 7.4 to about 7.6, about 7.0 to about 7.2, about 7.2 to about 7.4, about 7.4 to about 7.6, about 7.6 to about 7.8, about 7.8 to about 8.0, about 6.8, about 6.9, about 7.0, about 7.1, about 7.2, about 7.3, about 7.4, about 7.5, about 7.6, about 7.7, about 7.8, about 7.9, and/or about 8.0.

In some embodiments, the liquid formulation has a pH of about 7.5.

In some embodiments, the liquid pharmaceutical formulation comprises about 20 μg/mL fusion protein of SEQ ID NO: 11, about 50 mM alanine, about 200 mM trehalose, about 25 mM glycylglycine, about 0.02% polysorbate 20 (v/v), and about 10 μM DTPA, wherein the formulation has a pH of about 7.5.

In some embodiments, the liquid formulation is a reconstituted formulation.

In some embodiments, the reconstituted formulation is reconstituted from a lyophilized formulation.

The stable solid pharmaceutical formulations comprise about 0.01% to about 0.03% (w/w) IL12-ABD fusion protein of SEQ ID NO: 11, about 2% to about 8% alanine (w/w), about 50% to about 95% trehalose (w/w), about 2% to about 8% glycylglycine (w/w), about 0.1% to about 0.4% polysorbate 20 (w/w), and about 0.002% to about 0.006% diethylenetriaminepentaacetic acid (DTPA) (w/w).

In some embodiments, the solid pharmaceutical formulation comprises about 0.024 to about 0.028% (w/w) fusion protein of SEQ ID NO: 11, about 4.0% and about 5.0% alanine (w/w), about 70% to about 80% trehalose (w/w), about 3.0% to about 4.0% glycylglycine (w/w), about 0.15% to about 0.30% polysorbate 20 (w/w), and about 0.0045% to about 0.0055% DTPA (w/w).

In some embodiments, the solid formulation is a lyophilized formulation.

In some embodiments, the formulation is suitable for parenteral administration.

In some embodiments, the formulation is suitable for intravenous administration.

In some embodiments, the formulation is suitable for subcutaneous administration.

The present disclosure also provides a method of treating an IL-12-related disease or disorder in a subject in need thereof, the method comprising administering a formulation as disclosed herein to the subject.

### BRIEF DESCRIPTION OF THE DRAWINGS

FIG. 1 shows the sequence of an albumin binding domain included in the subject albumin binding domain fusion protein described herein. Included in the figure are the variable heavy domain and variable light domain sequences, as well as vhCDR1-3 and vICDR1-3 sequences.

FIG. 2 provides the amino acid sequence of an IL-12 cytokine included in the ABD fusion protein described herein. Also depicted in FIG. 2 is an IL12-ABD fusion protein where the ABD is A10m3.

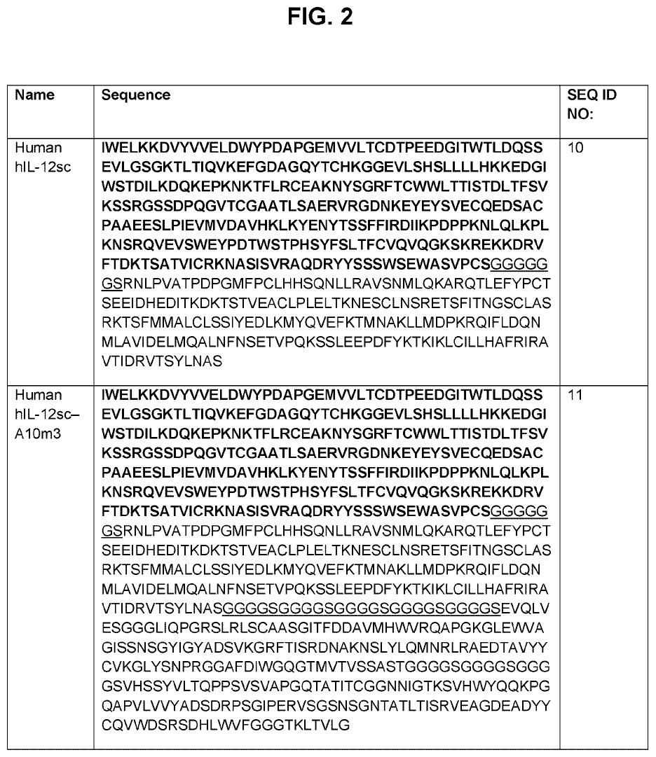

### DETAILED DESCRIPTION

### 1.1 Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a formulation” includes a plurality of such formulations, so that a formulation X includes a plurality of formulations X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit.

A “stable” IL12-ABD fusion protein formulation is one in which IL12-ABD fusion protein substantially retains its physical stability and/or chemical stability and/or its biological activity upon storage. The storage period is generally selected based on the intended shelf-life of the formulation. Various analytical techniques for measuring protein stability are available in the art. Examples of the analytical techniques are described below. By “substantially retains” is intended 85% or greater retention, such as at least 90% retention or at least 95% retention.

An IL12-ABD fusion protein “retains its physical stability” in a pharmaceutical formulation if it shows no significant physical changes such as aggregation, precipitation and/or denaturation upon visual examination of color and/or clarity, or as measured by UV light scattering or by size exclusion chromatography.

An IL12-ABD fusion protein “retains its chemical stability” in a pharmaceutical formulation if no significant chemical changes of the protein is shown. Chemical stability can be assessed by detecting and quantifying chemically altered forms of the protein. Chemical alteration may involve size modification (e.g., clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g., occurring as a result of deamidation, oxidation and/or isomerization) which can be evaluated by ion-exchange chromatography, for example.

An IL12-ABD fusion protein “retains its biological activity” in a pharmaceutical formulation if the biological activity of the protein at a given time is not significantly changed from the biological activity exhibited at the time the pharmaceutical formulation was prepared. “Biological activity” of an IL12-ABD fusion protein refers to the ability of the protein to produce a measurable biological response which can be measured in vitro or in vivo.

A “pharmaceutical formulation” refers to a preparation which contains no components in amounts that are unacceptably toxic to the patient to which the formulation would be administered.

Percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared. Unless otherwise specified, the percent sequence identity is determined using BLAST algorithms using default parameters.

By “subject” is meant a human or non-human mammal, including, but not limited to, bovine, equine, canine, ovine, feline, and rodent, including murine and rattus species, subjects. A “patient” is a human subject.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±1%, ±5%, or ±10%, of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals, substituents, and ranges. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2,” implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10,” it implies a continuous range that includes whole numbers and fractional numbers less than number 10, as discussed above.

Similarly, if the variable disclosed is a number greater than “number 10,” it implies a continuous range that includes whole numbers and fractional numbers greater than number 10.

These ranges can be modified by the term “about,” whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

### 1.2. IL12-ABD Fusion Proteins

Interleukin 12 (also known as IL-12 or IL12) refers to an interleukin that is a heterodimeric cytokine encoded by the IL-12A and IL-12B genes (Genbank Accession numbers: NM_000882 (IL-12A) and NM_002187 (IL-12B)). IL-12 is composed of a bundle of four alpha helices and is involved in the differentiation of native T cells into TH1 cells. IL-12 binds to the IL-12 receptor, which is a heterodimeric receptor formed by IL-12R-31 and IL-12R-B2. IL-12 is known as a T cell-stimulating factor that can stimulate the growth and function of T cells. In particular, IL-12 can stimulate the production of interferon gamma (IFN-γ), and tumor necrosis factor-alpha (TNF-α) from T cells and natural killer (NK) cells and reduce IL-4 mediated suppression of IFN-γ. IL-12 can further mediate enhancement of the cytotoxic activity of NK cells and CD8+ cytotoxic T lymphocytes. Moreover, IL-12 can also have anti-angiogenic activity by increasing production of interferon gamma, which in turn increases the production of the chemokine inducible protein-10 (IP-10 or CXCL10). IP-10 then mediates this anti-angiogenic effect. Without being bound by any particular theory of operation, it is believed that IL-12 through its ability to induce immune responses and its anti-angiogenic activity can be used to treat cancers.

In an embodiment, the IL-12 is a single chain IL-12 polypeptide comprising an IL-12 p35 subunit attached to an IL-12 p40 subunit. Such IL-12 single chain polypeptides advantageously retain one or more of the biological activities of wildtype IL-12. In some embodiments, the single chain IL-12 polypeptide described herein is according to the formula, from N-terminus to C-terminus, (p40)-(L)-(p35), wherein “p40” is an IL-12 p40 subunit, “p35” is IL-12 p35 subunit and L is a linker. In other embodiments, the single chain IL-12 is according to the formula from N-terminus to C-terminus, (p35)-(L)-(p40). Any suitable linker can be used in the single chain IL-12 polypeptide including those described herein. Suitable linkers can include, for example, linkers having the amino acid sequence (GGGGS)ₓ wherein x is an integer from 1-10. Other suitable linkers include, for example, the amino acid sequence GGGGGGS. Exemplary single chain IL-12 linkers than can be used with the subject single chain IL-12 polypeptides are also described in Lieschke et al., Nature Biotechnology 15:35-40 (1997), which is incorporated herein in its entirety by reference and particularly for its teaching of IL-12 polypeptide linkers.

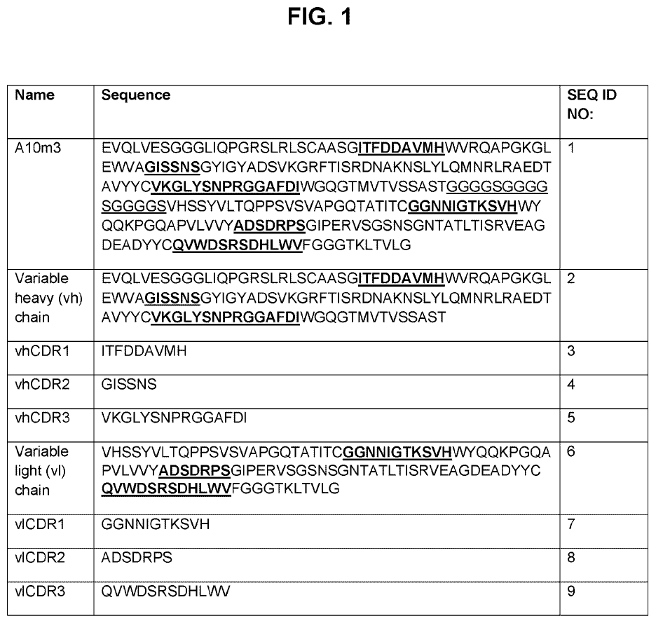

The IL12-ABD fusion proteins provided herein include an albumin binding domain attached to an IL12 fusion partner via a linker. As discussed herein, subject ABD fusion proteins are able to undergo FcRn mediated endosomal recycling and, thus, advantageously exhibit extended half life compared to counterparts that do not include such ABDs. The amino acids sequences of the ABDs, including vhCDR1, vhCDR2, vhCDR3, VICDR1, VICDR2, vICDR3, variable heavy chain and variable light chain sequences, are disclosed, for example, in FIG. 1

### 1.3. Formulation

In one aspect, provided herein is an aqueous liquid or a solid formulation (pharmaceutical composition), such as a lyophilized formulation, comprising an IL12-ABD fusion protein. In an embodiment, the formulation is a reconstituted formulation that has been reconstituted from a lyophilized formulation. In an embodiment, the formulation is sterile. In an embodiment, the formulation is stable. In an embodiment, the formulation further comprises at least one free amino acid. In an embodiment, the formulation further comprises at least one surfactant. In an embodiment, the formulation further comprises at least one buffering component. In an embodiment, the formulation further comprises at least one chelator. In an embodiment, the formulation comprises at least one saccharide. In an embodiment, the formulation comprises IL12-ABD fusion protein. In an embodiment, the formulation comprises at least one free amino acid, at least one surfactant, at least one buffering component, at least one chelator, at least one saccharide, and IL12-ABD fusion protein.

In an embodiment, the liquid formulation comprises about 10 μg/mL to about 50 μg/mL IL12-ABD fusion protein, about 25 mM to about 100 mM alanine, about 100 mM to about 400 mM trehalose, about 10 mM to about 50 mM glycylglycine, about 0.01% to about 0.04% polysorbate 20 (v/v), and about 5 μM to about 20 μM diethylenetriaminepentaacetic acid (DTPA), wherein the formulation has a pH of about 6.8 to about 8.0. In an embodiment, the liquid formulation comprises about 20 μg/mL to about 50 μg/mL IL12-ABD fusion protein, about 50 mM alanine, about 200 mM trehalose, about 25 mM glycylglycine, about 0.02% polysorbate 20 (v/v), and about 10 μM DTPA, wherein the formulation has a pH of about 7.5. In an embodiment, the liquid formulation comprises about 20 μg/mL IL12-ABD fusion protein, about 50 mM alanine, about 200 mM trehalose, about 25 mM glycylglycine, about 0.02% polysorbate 20 (v/v), and about 10 μM DTPA, wherein the formulation has a pH of about 7.5.

In an embodiment, the solid formulation comprises about 0.01% to about 0.03% (w/w) fusion protein, about 2% to about 8% alanine (w/w), about 50% to about 95% trehalose (w/w), about 2% to about 8% glycylglycine (w/w), about 0.1% to about 0.4% polysorbate 20 (w/w), and about 0.002% to about 0.006% diethylenetriaminepentaacetic acid (DTPA) (w/w). In an embodiment, the solid formulation comprises about 0.024 to about 0.028% (w/w) fusion protein, about 4.0% and about 5.0% alanine (w/w), about 70% to about 80% trehalose (w/w), about 3.0% to about 4.0% glycylglycine (w/w), about 0.15% to about 0.30% polysorbate 20 (w/w), and about 0.0045% to about 0.0055% DTPA (w/w). In an embodiment, the weight percentages are w/w of the lyophilized formulation.

### 1.3.1. IL12-ABD Fusion Protein Concentrations

In an embodiment, the IL12-ABD concentration in the liquid formulation is about 10 μg/mL to about 50 μg/mL, about 10 μg/mL to about 30 μg/mL, about 15 μg/mL to about 25 μg/mL, about 10 μg/mL to about 15 μg/mL, about 15 μg/mL to about 20 μg/mL, about 20 μg/mL to about 25 μg/mL, about 25 μg/mL to about 30 μg/mL, about 30 μg/mL to about 35 μg/mL, about 35 μg/mL to about 40 μg/mL, about 40 μg/mL to about 45 μg/mL, about 45 μg/mL to about 50 μg/mL, about 10 μg/mL, about 15 μg/mL, about 20 μg/mL, about 25 μg/mL, about 30 μg/mL, about 35 μg/mL, about 40 μg/mL, about 45 μg/mL, and/or about 50 μg/mL.

In an embodiment, the IL12-ABD concentration in the liquid formulation is about 10 μg/mL to about 50 μg/mL. In an embodiment, the IL12-ABD concentration in the liquid formulation is about 10 μg/mL to about 30 μg/mL. In an embodiment, the IL12-ABD concentration in the liquid formulation is about 20 μg/mL.

In an embodiment, the IL12-ABD concentration in the solid formulation is about 0.01% to about 0.03% (w/w), about 0.015% to about 0.03% (w/w), about 0.02% to about 0.03% (w/w), about 0.024% to about 0.028% (w/w), about 0.01% to about 0.015% (w/w), about 0.015% to about 0.02% (w/w), about 0.02% to about 0.022% (w/w), about 0.022% to about 0.024% (w/w), about 0.024% to about 0.026% (w/w), about 0.026% to about 0.028% (w/w), about 0.028% to about 0.03% (w/w), about 0.01% (w/w), about 0.015% (w/w), about 0.02% (w/w), about 0.022% (w/w), about 0.024% (w/w), about 0.025% (w/w), about 0.026% (w/w), about 0.028% (w/w), and/or about 0.03% (w/w).

In an embodiment, the IL12-ABD concentration in the solid formulation is about 0.01% to about 0.03% (w/w). In an embodiment, the IL12-ABD concentration in the solid formulation is about 0.024% to about 0.028% (w/w). In an embodiment, the IL12-ABD concentration in the solid formulation is about 0.026% (w/w).

In an embodiment, the IL12-ABD amount in the liquid or solid formulation is at least 10 μg, such as at least 20 μg, at least 30 μg, at least 40 μg, at least 50 μg, at least 60 μg, at least 80 μg, at least 90 μg, at least 100 μg, at least 110 μg, at least 120 μg, at least 130 μg, at least 140 μg, at least 150 μg, at least 160 μg, at least 180 μg, or at least 200 μg. In an embodiment, the IL12-ABD amount in the liquid or solid formulation is about 10 μg to about 200 μg, about 20 μg to about 180 μg, about 30 μg to about 160 μg, about 40 μg to about 150 μg, about 50 μg to about 140 μg, about 60 μg to about 130 μg, about 80 μg to about 120 μg, about 90 μg to about 110 μg, about 10 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 160 μg, about 160 μg to about 180 μg, about 180 μg to about 200 μg, about 10 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 180 μg, and/or about 200 μg.

### 1.3.2. Chelators

In an embodiment, the formulation comprises a chelator.

In an embodiment, the chelator in the liquid formulation is at a concentration of about 5 μM to about 20 μM, about 5 μM to about 15 μM, about 5 μM to about 10 μM, about 5 μM to about 10 μM, about 10 μM to about 15 μM, about 15 μM to about 20 μM, about 5 μM, about 10 μM, about 15 μM, and/or about 20 μM, preferably about 10 μM.

In an embodiment, the chelator in the solid formulation is at a concentration of about 0.002% to about 0.006% (w/w), about 0.003% to about 0.006% (w/w), about 0.004% to about 0.006% (w/w), about 0.0045% to about 0.0055% (w/w), about 0.002% to about 0.003% (w/w), about 0.003% to about 0.004% (w/w), about 0.004% to about 0.005% (w/w), about 0.005% to about 0.006% (w/w), about 0.002% (w/w), about 0.003% (w/w), about 0.004% (w/w), about 0.0045% (w/w), about 0.005% (w/w), about 0.0055% (w/w), and/or about 0.0066% (w/w), preferably about 0.005% (w/w).

In an embodiment, the chelator is a naturally occurring compound. In an embodiment, the chelator is a synthetic compound. In an embodiment, the chelator is diethylenetriamine pentaacetic acid (DTPA).

### 1.3.3. Surfactants

In an embodiment, the formulation comprises a surfactant.

The surfactant can lower surface tension of a liquid. In an embodiment, the surfactant is a nonionic surfactant. Examples of surfactants include polysorbate (polyoxyethylene sorbitan monolaurate, for example, polysorbate 20 and polysorbate 80); TRITON (t-octylphenoxypolyethoxyethanol); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; sorbitan monopalmitate; the MONAQUAT series (Mona Industries, Inc., Paterson, N.J.); polyethyl glycol (PEG), polypropylene glycol (PPG), and copolymers of poloxyethylene and poloxypropylene glycol (e.g. Pluronics/Poloxamer, PF68 etc.). In an embodiment, the surfactant is a polysorbate. In an embodiment, the polysorbate is polysorbate 20, polysorbate 40, polysorbate 60, or polysorbate 80.

In an embodiment, the liquid formulation comprises a surfactant at a concentration of about 0.01% to about 0.04%, about 0.01% to about 0.03%, about 0.01% to about 0.02%, about 0.02% to about 0.03%, about 0.03% to about 0.04%, about 0.01%, about 0.02%, about 0.03%, and/or about 0.04% (w/v), preferably about 0.02% (w/v).

In an embodiment, the solid formulation comprises a surfactant at a concentration of about 0.1% to about 0.4%, about 0.15% to about 0.3%, about 0.1% to about 0.15%, about 0.15% to about 0.2%, about 0.2% to about 0.3%, about 0.3% to about 0.4%, about 0.1%, about 0.15%, about 0.2%, about 0.3%, and/or about 0.4% (w/w), preferably about 0.2% (w/w).

### 1.3.4. Polysorbates

In an embodiment, the formulation comprises a polysorbate.

### 1.3.4.1. Polysorbate 80

In an embodiment, the formulation comprises polysorbate 80 (PS80).

In an embodiment, the liquid formulation comprises about 0.01% to about 0.04%, about 0.01% to about 0.03%, about 0.01% to about 0.02%, about 0.02% to about 0.03%, about 0.03% to about 0.04%, about 0.01%, about 0.02%, about 0.03%, and/or about 0.04% (w/v) polysorbate 80. In an embodiment, the formulation comprises about 0.02% (w/v) polysorbate 80.

In an embodiment, the solid formulation comprises about 0.1% to about 0.4%, about 0.15% to about 0.3%, about 0.1% to about 0.15%, about 0.15% to about 0.2%, about 0.2% to about 0.3%, about 0.3% to about 0.4%, about 0.1%, about 0.15%, about 0.2%, about 0.3%, and/or about 0.4% (w/w) polysorbate 80. In an embodiment, the formulation comprises about 0.2% (w/w) polysorbate 80.

### 1.3.4.2. Polysorbate 60

In an embodiment, the formulation comprises polysorbate 60 (PS60).

In an embodiment, the liquid formulation comprises about 0.01% to about 0.04%, about 0.01% to about 0.03%, about 0.01% to about 0.02%, about 0.02% to about 0.03%, about 0.03% to about 0.04%, about 0.01%, about 0.02%, about 0.03%, and/or about 0.04% (w/v) polysorbate 60. In an embodiment, the formulation comprises about 0.02% (w/v) polysorbate 60.

In an embodiment, the solid formulation comprises about 0.1% to about 0.4%, about 0.15% to about 0.3%, about 0.1% to about 0.15%, about 0.15% to about 0.2%, about 0.2% to about 0.3%, about 0.3% to about 0.4%, about 0.1%, about 0.15%, about 0.2%, about 0.3%, and/or about 0.4% (w/w) polysorbate 60. In an embodiment, the formulation comprises about 0.2% (w/w) polysorbate 60.

### 1.3.4.3. Polysorbate 40

In an embodiment, the formulation comprises polysorbate 40 (PS40).

In an embodiment, the liquid formulation comprises about 0.01% to about 0.04%, about 0.01% to about 0.03%, about 0.01% to about 0.02%, about 0.02% to about 0.03%, about 0.03% to about 0.04%, about 0.01%, about 0.02%, about 0.03%, and/or about 0.04% (w/v) polysorbate 40. In an embodiment, the formulation comprises about 0.02% (w/V) polysorbate 40.

In an embodiment, the solid formulation comprises about 0.1% to about 0.4%, about 0.15% to about 0.3%, about 0.1% to about 0.15%, about 0.15% to about 0.2%, about 0.2% to about 0.3%, about 0.3% to about 0.4%, about 0.1%, about 0.15%, about 0.2%, about 0.3%, and/or about 0.4% (w/w) polysorbate 40. In an embodiment, the formulation comprises about 0.2% (w/w) polysorbate 40.

### 1.3.4.4. Polysorbate 20

In an embodiment, the formulation comprises polysorbate 20 (PS20).

In an embodiment, the liquid formulation comprises about 0.01% to about 0.04%, about 0.01% to about 0.03%, about 0.01% to about 0.02%, about 0.02% to about 0.03%, about 0.03% to about 0.04%, about 0.01%, about 0.02%, about 0.03%, and/or about 0.04% (v/v) polysorbate 20. In an embodiment, the formulation comprises about 0.02% (v/V) polysorbate 20.

In an embodiment, the solid formulation comprises about 0.1% to about 0.4%, about 0.15% to about 0.3%, about 0.1% to about 0.15%, about 0.15% to about 0.2%, about 0.2% to about 0.3%, about 0.3% to about 0.4%, about 0.1%, about 0.15%, about 0.2%, about 0.3%, and/or about 0.4% (w/w) polysorbate 20. In an embodiment, the formulation comprises about 0.2% (w/w) polysorbate 20.

### 1.3.5. Saccharides

In an embodiment, the liquid formulation comprises a saccharide and derivatives thereof, including monosaccharides, disaccharides, trisaccharides, polysaccharides, sugar alcohols, reducing sugars, nonreducing sugars, and the like. Examples of saccharides include glucose, mannose, trehalose, lactose, fructose, maltose, dextran, dextrin, erythritol, glycerol, arabitol, sylitol, sorbitol, mannitol, mellibiose, melezitose, raffinose, mannotriose, stachyose, maltose, lactulose, maltulose, glucitol, maltitol, lactitol, isomaltulose, and the like. In an embodiment, the saccharide is a disaccharide. In an embodiment, the disaccharide is trehalose. In an embodiment, the disaccharide is at a concentration from about 1 to about 100 mg/mL.

### 1.3.5.1. Trehalose

In an embodiment, the formulation comprises trehalose.

In an embodiment, the liquid formulation comprises about 100 mM to about 400 mM, about 100 mM to about 300 mM, about 150 mM to about 250 mM, about 100 mM to about 150 mM, about 150 mM to about 200 mM, about 200 mM to about 250 mM, about 250 mM to about 300 mM, about 300 mM to about 350 mM, about 350 mM to about 400 mM, about 100 mM, about 150 mM, about 200 mM, about 250 mM, about 300 mM, about 350 mM, and/or about 400 mM trehalose. In an embodiment, the liquid formulation comprises about 200 mM trehalose.

In an embodiment, the solid formulation comprises about 50% to about 95% trehalose, about 60% to about 95% trehalose, about 70% to about 95% trehalose, about 80% to about 95% trehalose, about 85% to about 90% trehalose, about 50% to about 60% trehalose, about 60% to about 70% trehalose, about 70% to about 80% trehalose, about 80% to about 85% trehalose, about 85% to about 90% trehalose, about 90% to about 95% trehalose, about 50% trehalose, about 60% trehalose, about 70% trehalose, about 80% trehalose, about 85% trehalose, about 90% trehalose, about 95% trehalose (w/w). In an embodiment, the solid formulation comprises about 70% to about 80% trehalose (w/w). In an embodiment, the solid formulation comprises about 75% trehalose (w/w).

### 1.3.6. Free Amino Acids

In an embodiment, the formulation comprises at least one free amino acid. In an embodiment, the formulation comprises only one free amino acid. In an embodiment, the formulation comprises only two free amino acids. In an embodiment, the formulation comprises only three free amino acids. The free amino acid can be in the L-form, the D-form or a mixture of these forms.

In an embodiment, the at least one free amino acid is glycine, alanine, valine, leucine, isoleucine, proline, serine, threonine, glutamine, asparagine, cysteine, methionine, phenylalanine, tyrosine, tryptophan, aspartic acid, glutamic acid, histidine, arginine, or lysine. In an embodiment, the at least one free amino acid is glycine, alanine, proline, serine, aspartic acid, arginine, or lysine.

In an embodiment, the free amino acid is in the liquid formulation at a concentration of about 25 mM to about 100 mM, about 25 mM to about 75 mM, about 40 mM to about 60 mM, about 25 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, about 90 mM to about 100 mM, about 25 mM, about 30 mM, about 40 mM, about 50 mM, about 60 mM, about 70 mM, about 80 mM, about 90 mM, and/or about 100 mM. In an embodiment, the free amino acid is in the liquid formulation at a concentration of about 50 mM.

In an embodiment, the free amino acid is in the solid formulation at a concentration of about 2% to about 8%, about 3% to about 7%, about 3% to about 6%, about 4% to about 5%, about 4.2% to about 4.8%, about 4.4% to about 4.6%, about 2% to about 3%, about 3% to about 4%, about 4% to about 4.1%, about 4.1% to about 4.2%, about 4.2% to about 4.3%, about 4.3% to about 4.4%, about 4.4% to about 4.5%, about 4.5% to about 4.6%, about 4.6% to about 4.7%, about 4.7% to about 4.8%, about 4.8% to about 4.9%, about 4.9% to about 5%, about 2%, about 3%, about 4%, about 4.1%, about 4.2%, about 4.3%, about 4.4%, about 4.45%, about 4.5%, about 4.6%, about 4.7%, about 4.8%, about 4.9%, about 5%, about 6%, about 7%, and/or about 8% (w/w). In an embodiment, the free amino acid is in the solid formulation at a concentration of about 4% and about 5% (w/w). In an embodiment, the free amino acid is in the solid formulation at a concentration of about 4.45% (w/w).

In an embodiment, the formulation comprises alanine and at least one additional free amino acid selected from glycine, proline, serine, aspartic acid, arginine, and lysine. In an embodiment, the formulation comprises alanine and arginine.

### 1.3.6.1. Alanine

In an embodiment, the formulation comprises alanine

In an embodiment, the liquid formulation comprises about 25 mM to about 100 mM, about 25 mM to about 75 mM, about 40 mM to about 60 mM, about 25 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, about 90 mM to about 100 mM, about 25 mM, about 30 mM, about 40 mM, about 50 mM, about 60 mM, about 70 mM, about 80 mM, about 90 mM, and/or about 100 mM alanine. In an embodiment, the liquid formulation comprises about 50 mM alanine.

In an embodiment, the solid formulation comprises about 2% to about 8%, about 3% to about 7%, about 3% to about 6%, about 4% to about 5%, about 4.2% to about 4.8%, about 4.4% to about 4.6%, about 2% to about 3%, about 3% to about 4%, about 4% to about 4.1%, about 4.1% to about 4.2%, about 4.2% to about 4.3%, about 4.3% to about 4.4%, about 4.4% to about 4.5%, about 4.5% to about 4.6%, about 4.6% to about 4.7%, about 4.7% to about 4.8%, about 4.8% to about 4.9%, about 4.9% to about 5%, about 2%, about 3%, about 4%, about 4.1%, about 4.2%, about 4.3%, about 4.4%, about 4.45%, about 4.5%, about 4.6%, about 4.7%, about 4.8%, about 4.9%, about 5%, about 6%, about 7%, and/or about 8% (w/w) alanine (w/w). In an embodiment, the solid formulation comprises about 4% and about 5% alanine (w/w). In an embodiment, the solid formulation comprises about 4.45% alanine (w/w).

### 1.3.6.2. Arginine

In an embodiment, the formulation comprises arginine.

In an embodiment, the liquid formulation comprises about 25 mM to about 100 mM, about 25 mM to about 75 mM, about 40 mM to about 60 mM, about 25 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, about 90 mM to about 100 mM, about 25 mM, about 30 mM, about 40 mM, about 50 mM, about 60 mM, about 70 mM, about 80 mM, about 90 mM, and/or about 100 mM arginine. In an embodiment, the liquid formulation comprises about 50 mM arginine.

In an embodiment, the solid formulation comprises about 2% to about 8%, about 3% to about 7%, about 3% to about 6%, about 4% to about 5%, about 4.2% to about 4.8%, about 4.4% to about 4.6%, about 2% to about 3%, about 3% to about 4%, about 4% to about 4.1%, about 4.1% to about 4.2%, about 4.2% to about 4.3%, about 4.3% to about 4.4%, about 4.4% to about 4.5%, about 4.5% to about 4.6%, about 4.6% to about 4.7%, about 4.7% to about 4.8%, about 4.8% to about 4.9%, about 4.9% to about 5%, about 2%, about 3%, about 4%, about 4.1%, about 4.2%, about 4.3%, about 4.4%, about 4.45%, about 4.5%, about 4.6%, about 4.7%, about 4.8%, about 4.9%, about 5%, about 6%, about 7%, and/or about 8% (w/w) arginine (w/w). In an embodiment, the solid formulation comprises about 4% and about 5% arginine (w/w). In an embodiment, the solid formulation comprises about 4.45% arginine (w/w).

### 1.3.7. Buffering Agents

In an embodiment, the formulation comprises at least one buffering agent (buffering component). Typically, the buffering agent, when present, is used to adjust the pH of the formulation to about 6.8 to about 8.0, about 7.0 to about 7.8, about 7.2 to about 7.8, about 7.4 to about 7.6, about 6.8 to about 7.0, about 7.0 to about 7.2, about 7.2 to about 7.4, about 7.4 to about 7.6, about 7.6 to about 7.8, about 7.8 to about 8.0, about 6.8, about 6.9, about 7.0, about 7.1, about 7.2, about 7.3, about 7.4, about 7.5, about 7.6, about 7.7, about 7.8, about 7.9, and/or about 8.0.

In an embodiment the at least one buffering agent is selected from acetate, succinate, gluconate, histidine, citrate, phosphate, maleate, cacodylate, tris(hydroxymethyl)aminomethane (Tris), 2-[N-morpholino] ethanesulfonic acid (MES), bis(2-hydroxyethyl)iminotris [hydroxymethyl]methane (Bis-Tris), N-[2-acetamido]-2-iminodiacetic acid (ADA), glycylglycine and other organic acid buffers. In an embodiment, the buffering agent is glycylglycine, Tris, histidine, citrate, phosphate, succinate, or acetate.

In an embodiment, the buffering component is in the liquid formulation at a concentration from about 10 mM to about 50 mM, about 10 mM to about 40 mM, about 10 mM to about 30 mM, about 10 mM to about 15 mM, about 15 mM to about 20 mM, about 20 mM to about 25 mM, about 25 mM to about 30 mM, about 35 mM to about 40 mM, about 40 mM to about 45 mM, about 45 mM to about 50 mM, about 10 mM, about 15 mM, about 20 mM, about 25 mM, about 30 mM, about 35 mM, about 40 mM, about 45 mM, and/or about 50 mM. In an embodiment, the buffering component is in the liquid formulation at a concentration of about 25 mM.

In an embodiment, the buffering component is in the solid formulation at a concentration from about 2% to about 8%, about 3% to about 7%, about 3% to about 6%, about 3% to about 4%, about 3.2% to about 3.6%, about 2% to about 3%, about 3% to about 3.1%, about 3.1% to about 3.2%, about 3.2% to about 3.3%, about 3.3% to about 3.4%, about 3.4% to about 3.5%, about 3.5% to about 3.6%, about 3.6% to about 3.7%, about 3.7% to about 3.8%, about 3.8% to about 3.9%, about 3.9% to about 4%, about 4% to about 5%, about 5% to about 6%, about 6% to about 7%, about 7% to about 8%, about 2%, about 3%, about 3.1%, about 3.2%, about 3.3%, about 3.4%, about 3.5%, about 3.6%, about 3.7%, about 3.8%, about 3.9%, about 4%, about 5%, about 6%, about 7%, and/or about 8% (w/w). In an embodiment, the buffering component is in the solid formulation at a concentration of about 3% to about 4% (w/w). In an embodiment, the buffering component is in the solid formulation at a concentration of about 3.4% (w/w).

In an embodiment, the buffering agent is glycylglycine. In an embodiment, the formulation comprises glycylglycine.

In an embodiment, the liquid formulation comprises about 10 mM to about 40 mM, about 10 mM to about 30 mM, about 10 mM to about 15 mM, about 15 mM to about 20 mM, about 20 mM to about 25 mM, about 25 mM to about 30 mM, about 35 mM to about 40 mM, about 40 mM to about 45 mM, about 45 mM to about 50 mM, about 10 mM, about 15 mM, about 20 mM, about 25 mM, about 30 mM, about 35 mM, about 40 mM, about 45 mM, and/or about 50 mM glycylglycine. In an embodiment, the liquid formulation comprises about 25 mM glycylglycine.

In an embodiment, the solid formulation comprises about 2% to about 8%, about 3% to about 7%, about 3% to about 6%, about 3% to about 4%, about 3.2% to about 3.6%, about 2% to about 3%, about 3% to about 3.1%, about 3.1% to about 3.2%, about 3.2% to about 3.3%, about 3.3% to about 3.4%, about 3.4% to about 3.5%, about 3.5% to about 3.6%, about 3.6% to about 3.7%, about 3.7% to about 3.8%, about 3.8% to about 3.9%, about 3.9% to about 4%, about 4% to about 5%, about 5% to about 6%, about 6% to about 7%, about 7% to about 8%, about 2%, about 3%, about 3.1%, about 3.2%, about 3.3%, about 3.4%, about 3.5%, about 3.6%, about 3.7%, about 3.8%, about 3.9%, about 4%, about 5%, about 6%, about 7%, and/or about 8% glycylglycine (w/w). In an embodiment, the solid formulation comprises about 3% to about 4% glycylglycine (w/w). In an embodiment, the solid formulation comprises about 3.4% glycylglycine (w/w).

1.3.7.1. pH

In an embodiment, the liquid formulation has a pH of about 6.8 to about 8.0, such as about 7.0 to about 7.8, about 7.2 to about 7.8, about 7.4 to about 7.6, about 6.8 to about 7.0, about 7.0 to about 7.2, about 7.2 to about 7.4, about 7.4 to about 7.6, about 7.6 to about 7.8, about 7.8 to about 8.0, about 6.8, about 7.0, about 7.1, about 7.2, about 7.3, about 7.4, about 7.5, about 7.6, about 7.7, about 7.8, about 7.9, and/or about 8.0. In an embodiment, the formulation has a pH of about 7.5.

### 1.3.8. Stabilization

In an embodiment, the liquid formulation stabilizes the IL12-ABD fusion protein from thermal stress. In an embodiment, the liquid formulation reduces the formation of sub-visible particles, as measured by FlowCam after thermal stress at 40° C. for fourteen days. In an embodiment, the liquid formulation reduces the formation of high molecular weight (HMW) species, as measured by size exclusion-high-performance liquid chromatography (SEC-HPLC) after thermal stress at 40° C. for fourteen days. In an embodiment, the liquid formulation reduces the formation of low molecular weight (LMW) species, as measured by size exclusion-high-performance liquid chromatography (SEC-HPLC) after thermal stress at 40° C. for fourteen days.

In an embodiment, the liquid formulation stabilizes the IL12-ABD fusion protein from freeze-thaw stress. In an embodiment, the liquid formulation reduces the formation of sub-visible particles, as measured by FlowCam after freeze-thaw stress from five freeze-thaw cycles. In an embodiment, the liquid formulation reduces the formation of high molecular weight (HMW) species, as measured by size exclusion-high-performance liquid chromatography (SEC-HPLC) after freeze-thaw stress from five freeze-thaw cycles. In an embodiment, the liquid formulation reduces the formation of low molecular weight (LMW) species, as measured by size exclusion-high-performance liquid chromatography (SEC-HPLC) after freeze-thaw stress from five freeze-thaw cycles.

In an embodiment, the liquid formulation stabilizes the IL12-ABD fusion protein from shear stress. In an embodiment, the liquid formulation reduces the formation of sub-visible particles, as measured by FlowCam after simulated shear stress for 24 hours at 900 RPM. In an embodiment, the liquid formulation reduces the formation of high molecular weight (HMW) species, as measured by size exclusion-high-performance liquid chromatography (SEC-HPLC) after simulated shear stress for 24 hours at 900 RPM. In an embodiment, the liquid formulation reduces the formation of low molecular weight (LMW) species, as measured by size exclusion-high-performance liquid chromatography (SEC-HPLC) after simulated shear stress for 24 hours at 900 RPM.

### 1.4. Administration of the Formulation

The suitable routes of administration for the IL12-ABD fusion protein formulations described herein include are but not limited to, parenterally (such as by subcutaneous, intravenous, intramuscular, intradermal, or intrasternal injection or infusion (e.g., as sterile injectable aqueous or non-aqueous solutions or suspensions, etc.)). In certain embodiments, the formulations are suitable for parenteral injection. In certain embodiments, the formulations are suitable for intravenous injection. In certain embodiments, the formulations are suitable for subcutaneous injection.

In some embodiments, the formulations are suitable for administration in single or multiple doses.

### 1.5. Dosage Form

In another aspect, provided herein are dosage forms containing one or more unit doses of a pharmaceutical composition comprising an IL12-ABD fusion protein.

In various embodiments, the dosage form is a pre-filled syringe. In various embodiments, the dosage form is an auto-inject pen.

In various embodiments, the dosage form comprises one or more unit doses of a formulation as described hereinabove. In typical embodiments, the formulation is a liquid formulation. In other embodiments, the formulation is a dry formulation, including but not limited to a lyophilate. In particular embodiments, the dosage form comprises a dry formulation and a measured quantity of aqueous diluent.

In some embodiments, the unit dosage form comprises a formulation comprising about 10 μg/mL to about 50 μg/mL IL12-ABD fusion protein, about 25 mM to about 100 mM alanine, about 100 mM to about 400 mM trehalose, about 10 mM to about 50 mM glycylglycine, about 0.01% to about 0.04% polysorbate 20 (v/v), and about 5 M to about 20 μM diethylenetriaminepentaacetic acid (DTPA), wherein the formulation has a pH of about 6.8 to about 8.0.

In some embodiments, the unit dosage form comprises a formulation comprising about 0.01% to about 0.03% (w/w) IL12-ABD fusion protein, about 2% to about 8% alanine (w/w), about 50% to about 95% trehalose (w/w), about 2% to about 8% glycylglycine (w/w), about 0.1% to about 0.4% polysorbate 20 (w/w), and about 0.002% to about 0.006% diethylenetriaminepentaacetic acid (DTPA) (w/w).

### 1.6. Methods of Treatment

In another aspect, provided herein are methods of treating a disease or disorder in a patient comprising administering to the patient the IL12-ABD fusion protein formulation described herein. In some embodiments, the patient has an IL-12 related disease or disorder such as cancer, an autoimmune disease, or an infectious disease. In some embodiments, the IL12-ABD fusion protein is administered as an immune adjuvant for the treatment of cancer, an autoimmune disease, or an infectious disease, such as administering the IL12-ABD fusion protein with a vaccine antigen.

In another aspect, provided herein are methods of inhibiting tumor growth in a patient comprising administering to the patient the IL12-ABD fusion protein formulation described herein.

In another aspect, provided herein are methods of treating cancer in a patient comprising administering to the patient the IL12-ABD fusion protein formulation described herein.

In an embodiment, the cancer to be treated includes, but is not limited to, carcinoma, blastoma, sarcoma, and certain leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, skin cancer/melanoma, as well as head and neck cancer and metastases associated with any of the primary tumors.

In another aspect, provided herein are methods of treating an autoimmune disease in a patient comprising administering to the patient the IL12-ABD fusion protein formulation described herein.

In an embodiment, the autoimmune disease to be treated includes, but is not limited to, rheumatoid arthritis, juvenile iodiopathic arthritis, and graft versus host disease (GvHD).

In another aspect, provided herein are methods of treating an infectious disease in a patient comprising administering to the patient the IL12-ABD fusion protein formulation described herein.

In an embodiment, the infectious disease to be treated includes, but is not limited to, a local or systemic viral infection such as encephalitis, influenza, common cold, immunodeficiency, and herpes viral infections. In an embodiment, the infectious disease is caused by a bacterium, virus, protozoan, helminth, or other microbial pathogen. In an embodiment, the infection to be treated is caused by a hepatitis virus, a human immunodeficiency virus (HIV), a human T-lymphotrophic virus (HTLV), a herpes virus, an Epstein-Barr virus, or a human papilloma virus.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXAMPLES

### Example 1: Stability Studies for Formulations with Varying Buffer and pH

Formulations of IL12-ABD fusion protein SONE-IL12-ABD (SEQ ID NO: 11) were prepared as shown in Table 1.

TABLE 1

| | | Conc. of SONE-IL12- |
|---|---|---|
| No. | Formulation | ABD (mg/ml) |
| A1 | 10 mM phosphate buffer, pH 7.0, 0.02% | 0.290 |
| | PS20 | |
| A2 | 10 mM phosphate buffer, pH 7.0 | 0.289 |
| A3 | 10 mM TRIS buffer, pH 7.5, 0.02% PS20 | 0.287 |
| A4 | 10 mM TRIS buffer, pH 7.5 | 0.287 |
| A5 | 10 mM histidine, pH 6.0, 0.02% PS20 | 0.282 |
| A6 | 10 mM histidine, pH 6.0 | 0.281 |
| A7 | water, 0.02% PS20 | 0.244 |
| A8 | water | 0.245 |
| A9 | 10 mM acetate buffer, pH 5.5, 0.02% | 0.289 |
| | PS20 | |
| A10 | 10 mM acetate buffer, pH 5.5 | 0.279 |
| A11 | 10 mM citrate buffer, pH 5.5, 0.02% | 0.303 |
| | PS20 | |
| A12 | 10 mM citrate buffer, pH 5.5 | 0.289 |
| A13 | 10 mM succinate buffer, pH 5.5, 0.02% | 0.294 |
| | PS20 | |
| A14 | 10 mM succinate buffer, pH 5.5 | 0.298 |
| A-CF | 20 mM TRIS buffer, 150 mM NaCl, | 0.300 |
| | 0.02% PS20, pH 7.2 | |

### 1.1. Thermal Stress Study

The formulations were thermally stressed for 7 days at 40° C. and monitored for osmolality, pH, A280, right angle light scattering (RALS), DLS, Flowcam, SEC-HPLC, and reducing and non-reducing SDS-page. The results for osmolality, pH, A280, and RALS are shown in Table 2.

TABLE 2

| | Osmolality | | | | Concentration | | | | | RALS | |
|---|---|---|---|---|---|---|---|---|---|---|---|
| | (mOsm/kg) | | pH | | T0 | | T7 | | % | (525 Volts) | |
| No. | T0 | T7 | T0 | T7 | A280 | mg/ml | A280 | mg/ml | Recovery | T0 | T7 |
| A1 | 29 | 32 | 7.19 | 7.16 | 0.445 | 0.29 | 0.483 | 0.315 | 109 | 0.917 | 0.839 |
| A2 | 30 | 32 | 7.19 | 7.21 | 0.443 | 0.289 | 0.484 | 0.316 | 109 | 1.032 | 0.846 |
| A3 | 15 | 16 | 7.82 | 7.72 | 0.439 | 0.287 | 0.436 | 0.285 | 99 | 1.051 | 0.914 |
| A4 | 15 | 16 | 7.83 | 7.75 | 0.439 | 0.287 | 0.437 | 0.285 | 100 | 1.124 | 0.948 |
| A5 | 9 | 9 | 6.32 | 6.34 | 0.432 | 0.282 | 0.476 | 0.311 | 110 | 1.359 | 3.845 |
| A6 | 9 | 8 | 6.33 | 6.34 | 0.43 | 0.281 | 0.452 | 0.295 | 105 | 1.322 | 3.722 |
| A7 | 2 | 0 | 5.41 | 6.31 | 0.374 | 0.244 | 0.389 | 0.254 | 104 | 0.766 | 2.328 |
| A8 | −1 | 0 | 5.47 | 6.28 | 0.376 | 0.245 | 0.372 | 0.243 | 99 | 1.057 | 2.531 |
| A9 | 19 | 17 | 5.82 | 5.88 | 0.442 | 0.289 | 0.473 | 0.309 | 107 | 1.220 | 9.999 |
| A10 | 18 | 19 | 5.81 | 5.86 | 0.427 | 0.279 | 0.491 | 0.32 | 115 | 1.254 | 9.999 |
| A11 | 40 | 41 | 5.6 | 5.7 | 0.464 | 0.303 | 0.463 | 0.302 | 100 | 1.080 | 2.845 |
| A12 | 41 | 46 | 5.62 | 5.69 | 0.442 | 0.289 | 0.491 | 0.32 | 111 | 0.951 | 2.698 |
| A13 | 31 | 32 | 5.67 | 5.67 | 0.451 | 0.294 | 0.492 | 0.321 | 109 | 1.064 | 8.237 |
| A14 | 31 | 31 | 5.67 | 5.68 | 0.456 | 0.298 | 0.483 | 0.315 | 106 | 1.039 | 8.376 |
| A-CF | 357 | 364 | 7.2 | 7.29 | 0.459 | 0.3 | 0.474 | 0.309 | 103 | 0.835 | 0.739 |

As shown in Table 2, concentration after incubation for 7 days at 40° C. increased the most for formulations containing acetate and citrate. Additionally, as assessed using RALS, higher pH formulations and formulations containing citrate (with three carboxyl groups) had less light scattering particles (i.e., lower RALS values) after thermal stress compared to the formulations containing acetate (with one carboxyl group) and formulations containing succinate (with two carboxyl groups).

The formulations were assessed by DLS after incubation for 7 days at 40° C. As shown in Table 3, particle size diameters increased for formulations with pH less than 7 after thermal stress.

TABLE 3

| | T0 | | | T7 | | |
|---|---|---|---|---|---|---|
| No. | PDI | P1 d (nm) | Vol % | PDI | P1 d (nm) | Vol % |
| A1 | 0.503 | 10.51 | 100 | 0.283 | 12.82 | 100 |
| A2 | 0.222 | 10.54 | 100 | 0.265 | 12.23 | 100 |
| A3 | 0.295 | 11.61 | 100 | 0.345 | 12.32 | 100 |
| A4 | 0.160 | 9.982 | 100 | 0.308 | 6.89 | 82.6 |
| A5 | 0.148 | 12.32 | 100 | 0.152 | 28.41 | 100 |
| A6 | 0.215 | 11.71 | 100 | 0.117 | 24.51 | 100 |
| A7 | 0.226 | 12.16 | 100 | 0.202 | 18.41 | 100 |
| A8 | 0.203 | 11.48 | 100 | 0.161 | 11.36 | 82.3 |
| A9 | 0.267 | 11.53 | 100 | 0.195 | 22.86 | 75.6 |
| A10 | 0.212 | 13.22 | 100 | 0.193 | 27.33 | 82.5 |
| A11 | 0.324 | 11.47 | 100 | 0.157 | 25.02 | 100 |
| A12 | 0.213 | 11.81 | 100 | 0.173 | 10.84 | 65.5 |
| A13 | 0.299 | 10.37 | 100 | 0.196 | 25.15 | 100 |
| A14 | 0.34 | 11.01 | 100 | 0.208 | 41.11 | 100 |
| A-CF | 0.152 | 9.42 | 100 | 0.233 | 9.86 | 100 |

The formulations were assessed for presence of sub-particulates by Flowcam after incubation for 7 days at 40° C. The results are shown in Table 4.

TABLE 4

| | T0 | | | T7 | | |
|---|---|---|---|---|---|---|
| No. | 2-10 um | 10-25 um | 25-50 um | 2-10 um | 10-25 um | 25-50 um |
| A1 | 1334 | 90 | 0 | 479 | 65 | 0 |
| A2 | 214 | 15 | 8 | 138 | 54 | 0 |
| A3 | 138 | 23 | 0 | 164 | 31 | 8 |
| A4 | 235 | 39 | 0 | 693 | 99 | 0 |
| A5 | 135 | 25 | 0 | 243 | 17 | 25 |
| A6 | 130 | 31 | 0 | 123 | 0 | 15 |
| A7 | 358 | 31 | 0 | 476 | 37 | 0 |
| A8 | 2114 | 38 | 8 | 976 | 57 | 0 |
| A9 | 436 | 8 | 15 | 1082 | 237 | 34 |
| A10 | 276 | 31 | 0 | 514 | 43 | 17 |
| A11 | 406 | 8 | 0 | 246 | 33 | 22 |
| A12 | 352 | 15 | 8 | 356 | 51 | 8 |
| A13 | 1057 | 61 | 8 | 1191 | 35 | 0 |
| A14 | 398 | 31 | 8 | 785 | 117 | 36 |
| A-CF | 115 | 15 | 8 | 758 | 54 | 0 |

The formulations were thermally stressed for 7 days at 40° C. and monitored by SEC-HPLC for total recovery, fusion protein recovery, and percent impurities consisting of HMW and LMW. The results are shown in Tables 5 and 6. At T7 after the thermal stress, the formulations at pH<7.0 shows higher % HMW species and lower % Main recovery. Formulations with higher pH>7.0 (A1-A4 and A-CF) were stable against thermal stress at T7 with the least amount of aggregation.

TABLE 5

| | T0 | | | | | | |
|---|---|---|---|---|---|---|---|
| No. | HMWAUC | MAIN AUC | LMW AUC | TOTAL AUC | % HMW | % MAIN | % LMW |
| A1 | 722 | 70360 | 2954 | 74035 | 0.97 | 95.04 | 3.99 |
| A2 | 801 | 70621 | 2705 | 74127 | 1.08 | 95.27 | 3.65 |
| A3 | 865 | 68561 | 2441 | 71867 | 1.20 | 95.40 | 3.40 |
| A4 | 869 | 68204 | 2272 | 71345 | 1.22 | 95.60 | 3.18 |
| A5 | 1021 | 65934 | 3523 | 70478 | 1.45 | 93.55 | 5.00 |
| A6 | 866 | 67373 | 2245 | 70484 | 1.23 | 95.59 | 3.19 |
| A7 | 1302 | 64671 | 2461 | 68434 | 1.90 | 94.50 | 3.60 |
| A8 | 930 | 63231 | 2696 | 66857 | 1.39 | 94.58 | 4.03 |
| A9 | 1018 | 66465 | 2813 | 70295 | 1.45 | 94.55 | 4.00 |
| A10 | 863 | 69291 | 3032 | 73185 | 1.18 | 94.68 | 4.14 |
| A11 | 945 | 68034 | 3087 | 72066 | 1.31 | 94.41 | 4.28 |
| A12 | 734 | 70844 | 3400 | 74979 | 0.98 | 94.49 | 4.53 |
| A13 | 893 | 67184 | 3340 | 71417 | 1.25 | 94.07 | 4.68 |
| A14 | 757 | 67286 | 3387 | 71430 | 1.06 | 94.20 | 4.74 |
| A-CF | 693 | 70626 | 3138 | 74457 | 0.93 | 94.85 | 4.21 |

TABLE 6

| | T7 | | | | | | | | |
|---|---|---|---|---|---|---|---|---|---|
| | | MAIN | LMW | TOTAL | % | % | % | % main | % total |
| No. | HMWAUC | AUC | AUC | AUC | HMW | MAIN | LMW | recovery | recovery |
| A1 | 17373 | 40899 | 4423 | 62696 | 27.71 | 65.23 | 7.06 | 58.13 | 84.68 |
| A2 | 17232 | 46021 | 5371 | 68624 | 25.11 | 67.06 | 7.83 | 65.17 | 92.58 |
| A3 | 27557 | 36461 | 6226 | 70243 | 39.23 | 51.91 | 8.86 | 53.18 | 97.74 |
| A4 | 27011 | 36679 | 6255 | 69945 | 38.62 | 52.44 | 8.94 | 53.78 | 98.04 |
| A5 | 68309 | 8077 | 2904 | 79289 | 86.15 | 10.19 | 3.66 | 12.25 | 112.5 |
| A6 | 68735 | 7729 | 1456 | 77921 | 88.21 | 9.92 | 1.87 | 11.47 | 110.55 |
| A7 | 45581 | 7298 | 4742 | 57620 | 79.11 | 12.66 | 8.23 | 11.28 | 84.2 |
| A8 | 45990 | 6541 | 2092 | 54623 | 84.20 | 11.97 | 3.83 | 10.34 | 81.7 |
| A9 | 60065 | 6869 | 1980 | 68914 | 87.16 | 9.97 | 2.87 | 10.33 | 98.03 |
| A10 | 53375 | 6209 | 1671 | 61255 | 87.14 | 10.14 | 2.73 | 8.96 | 83.7 |
| A11 | 56616 | 21869 | 3530 | 82015 | 69.03 | 26.66 | 4.3 | 32.14 | 113.8 |
| A12 | 52299 | 21677 | 2502 | 76478 | 68.38 | 28.34 | 3.27 | 30.6 | 102 |
| A13 | 64646 | 10658 | 2617 | 77921 | 82.96 | 13.68 | 3.36 | 15.86 | 109.11 |
| A14 | 42851 | 7498 | 1485 | 51834 | 82.67 | 14.47 | 2.86 | 11.14 | 72.57 |
| A-CF | 6566 | 59182 | 5627 | 71374 | 9.2 | 82.92 | 7.88 | 83.8 | 95.86 |

The formulations that were thermally stressed for 7 days at 40° C. were also analyzed by reducing and non-reducing SDS-PAGE. Each formulation was consistent with the A-CF on the reduced gels. Non-reduced gels showed more variability and extra HMW bands in A5 and A6 containing histidine with and without PS20, A7 and A8 containing water with and without PS20, A9 and A10 containing acetate with and without PS20, and A11 and A12 containing citrate with and without PS20.

### 1.2. Freeze-Thaw Study

The formulations at T0 were subjected to five freeze-thaws and then analyzed for osmolality, pH, RALS, A280, DLS, SEC-HPLC, and reducing and non-reducing SDS-page. The results for osmolality, pH, A280, and right angle light scattering (RALS) are shown in Table 7.

TABLE 7

| | Osmolality (mOsm/kg) | | pH | | Concentration | | | | | RALS (525 Volts) | |
|---|---|---|---|---|---|---|---|---|---|---|---|
| | | Freeze- | | Freeze | T0 | | Freeze-thaw | | % | | Freeze |
| No. | T0 | thaw | T0 | thaw | A280 | mg/ml | A280 | mg/ml | Recovery | T0 | thaw |
| A1 | 29 | 34 | 7.19 | 7.22 | 0.445 | 0.29 | 0.461 | 0.301 | 104 | 0.917 | 0.896 |
| A2 | 30 | 30 | 7.19 | 7.21 | 0.443 | 0.289 | 0.45 | 0.294 | 102 | 1.032 | 0.729 |
| A3 | 15 | 16 | 7.82 | 7.73 | 0.439 | 0.287 | 0.448 | 0.292 | 102 | 1.051 | 0.927 |
| A4 | 15 | 17 | 7.83 | 7.74 | 0.439 | 0.287 | 0.443 | 0.289 | 101 | 1.124 | 0.798 |
| A5 | 9 | 9 | 6.32 | 6.33 | 0.432 | 0.282 | 0.408 | 0.266 | 94 | 1.359 | 1.405 |
| A6 | 9 | 10 | 6.33 | 6.34 | 0.43 | 0.281 | 0.402 | 0.262 | 93 | 1.322 | 1.563 |
| A7 | 2 | 0 | 5.41 | 5.99 | 0.374 | 0.244 | 0.28 | 0.183 | 75 | 0.766 | 1.8 |
| A8 | −1 | 0 | 5.47 | 6.16 | 0.376 | 0.245 | 0.298 | 0.195 | 79 | 1.057 | 1.133 |
| A9 | 19 | 18 | 5.82 | 6.16 | 0.442 | 0.289 | 0.385 | 0.251 | 87 | 1.22 | 1.084 |
| A10 | 18 | 19 | 5.81 | 5.81 | 0.427 | 0.279 | 0.387 | 0.253 | 91 | 1.254 | 1.581 |
| A11 | 40 | 41 | 5.6 | 5.69 | 0.464 | 0.303 | 0.475 | 0.31 | 102 | 1.08 | 0.82 |
| A12 | 41 | 41 | 5.62 | 5.69 | 0.442 | 0.289 | 0.442 | 0.289 | 100 | 0.951 | 0.87 |
| A13 | 31 | 30 | 5.67 | 5.67 | 0.451 | 0.294 | 0.455 | 0.297 | 101 | 1.064 | 1.273 |
| A14 | 31 | 30 | 5.67 | 5.66 | 0.456 | 0.298 | 0.401 | 0.262 | 88 | 1.039 | 1.269 |
| A-CF | 357 | 387 | 7.20 | 7.22 | 0.459 | 0.3 | 0.458 | 0.299 | 100 | 0.835 | 0.694 |

As shown in Table 7, after freeze-thaw cycling, A7 and A8 containing water with and without PS20, A9 containing acetate with PS20, and A14 containing succinate had low percent recoveries below 90 percent for concentration. Additionally, as assessed using RALS, the trends are consistent with the temperature stress, where the lowest RALS values were for formulations with higher pH, along with formulations containing citrate.

The formulations were assessed by DLS after incubation after five freeze-thaw cycles. As shown in Table 8, the polydispersity index (PDI) increased for all formulations except for A-CF after five freeze-thaws.

TABLE 8

| | T0 | | | Freeze-thaw | | |
|---|---|---|---|---|---|---|
| No. | PDI | P1 | Vol % | PDI | P1 | Vol % |
| A1 | 0.503 | 10.51 | 100 | 0.297 | 8.578 | 88.1 |
| A2 | 0.222 | 10.54 | 100 | 0.313 | 10.87 | 100 |
| A3 | 0.295 | 11.61 | 100 | 0.465 | 10.91 | 99.9 |
| A4 | 0.16 | 9.982 | 100 | 0.688 | 11.01 | 100 |
| A5 | 0.148 | 12.32 | 100 | 0.690 | 11.66 | 99.6 |
| A6 | 0.215 | 11.71 | 100 | 0.663 | 12.05 | 99.8 |
| A7 | 0.226 | 12.16 | 100 | 0.343 | 13.45 | 99.2 |
| A8 | 0.203 | 11.48 | 100 | 0.458 | 11.68 | 99.7 |
| A9 | 0.267 | 11.53 | 100 | 0.744 | 11.78 | 99.9 |
| A10 | 0.212 | 13.22 | 100 | 0.653 | 11.74 | 99.5 |
| A11 | 0.324 | 11.47 | 100 | 0.394 | 9.775 | 99.9 |
| A12 | 0.213 | 11.81 | 100 | 1.000 | 8.789 | 100 |
| A13 | 0.299 | 10.37 | 100 | 0.562 | 10.79 | 99.8 |
| A14 | 0.34 | 11.01 | 100 | 0.785 | 10.43 | 99.8 |
| A-CF | 0.152 | 9.42 | 100 | 0.289 | 9.497 | 100 |

The formulations were subjected to five freeze-thaws and monitored by SEC-HPLC for total recovery, fusion protein recovery, and percent impurities consisting of HMW and LMW. The results are shown in Tables 9 and 10. The formulations are freeze-thaw sensitive. Consistent with the time and temperature effect (thermal stress study), the formulations at higher pH had relatively higher % recoveries for both main peak and total peak and had less increase in LMW species.

TABLE 9

| | T0 | | | | | | |
|---|---|---|---|---|---|---|---|
| No. | HMWAUC | MAIN AUC | LMW AUC | TOTAL AUC | % HMW | % MAIN | % LMW |
| A1 | 722 | 70360 | 2954 | 74035 | 0.97 | 95.04 | 3.99 |
| A2 | 801 | 70621 | 2705 | 74127 | 1.08 | 95.27 | 3.65 |
| A3 | 865 | 68561 | 2441 | 71867 | 1.20 | 95.40 | 3.40 |
| A4 | 869 | 68204 | 2272 | 71345 | 1.22 | 95.60 | 3.18 |
| A5 | 1021 | 65934 | 3523 | 70478 | 1.45 | 93.55 | 5.00 |
| A6 | 866 | 67373 | 2245 | 70484 | 1.23 | 95.59 | 3.19 |
| A7 | 1302 | 64671 | 2461 | 68434 | 1.9 | 94.50 | 3.60 |
| A8 | 930 | 63231 | 2696 | 66857 | 1.39 | 94.58 | 4.03 |
| A9 | 1018 | 66465 | 2813 | 70295 | 1.45 | 94.55 | 4.00 |
| A10 | 863 | 69291 | 3032 | 73185 | 1.18 | 94.68 | 4.14 |
| A11 | 945 | 68034 | 3087 | 72066 | 1.31 | 94.41 | 4.28 |
| A12 | 734 | 70844 | 3400 | 74979 | 0.98 | 94.49 | 4.53 |
| A13 | 893 | 67184 | 3340 | 71417 | 1.25 | 94.07 | 4.68 |
| A14 | 757 | 67286 | 3387 | 71430 | 1.06 | 94.20 | 4.74 |
| A-CF | 693 | 70626 | 3138 | 74457 | 0.93 | 94.85 | 4.21 |

TABLE 10

| | Freeze-thaw | | | | | | | | |
|---|---|---|---|---|---|---|---|---|---|
| | HMW | MAIN | LMW | TOTAL | % | % | % | % main | % total |
| No. | AUC | AUC | AUC | AUC | HMW | MAIN | LMW | recovery | recovery |
| A1 | 6803 | 46121 | 2686 | 55611 | 12.23 | 82.94 | 4.83 | 65.55 | 75.11 |
| A2 | 2600 | 60236 | 3558 | 66395 | 3.92 | 90.72 | 5.36 | 85.30 | 89.57 |
| A3 | 816 | 57701 | 2460 | 60977 | 1.34 | 94.63 | 4.03 | 84.16 | 84.85 |
| A4 | 679 | 59587 | 2961 | 63226 | 1.07 | 94.24 | 4.68 | 87.37 | 88.62 |
| A5 | 994 | 55013 | 3100 | 59106 | 1.68 | 93.07 | 5.25 | 83.44 | 83.87 |
| A6 | 970 | 55464 | 3228 | 59662 | 1.63 | 92.96 | 5.41 | 82.32 | 84.65 |
| A7 | 666 | 34920 | 1745 | 37331 | 1.78 | 93.54 | 4.67 | 54.00 | 54.55 |
| A8 | 789 | 35230 | 1272 | 37291 | 2.12 | 94.47 | 3.41 | 55.72 | 55.78 |
| A9 | 1060 | 48387 | 2862 | 52310 | 2.03 | 92.50 | 5.47 | 72.80 | 74.41 |
| A10 | 1047 | 49032 | 3212 | 53290 | 1.96 | 92.01 | 6.03 | 70.76 | 72.82 |
| A11 | 674 | 62333 | 3208 | 66215 | 1.02 | 94.14 | 4.85 | 91.62 | 91.88 |
| A12 | 408 | 61667 | 3257 | 65332 | 0.62 | 94.39 | 4.99 | 87.05 | 87.13 |
| A13 | 819 | 56449 | 3061 | 60329 | 1.36 | 93.57 | 5.07 | 84.02 | 84.47 |
| A14 | 789 | 57319 | 4089 | 62196 | 1.27 | 92.16 | 6.57 | 85.19 | 87.07 |
| A-CF | 330 | 64412 | 2413 | 67154 | 0.49 | 95.92 | 3.59 | 91.20 | 90.19 |

The formulations that were subjected to five freeze-thaws were also analyzed by reducing and non-reducing SDS-PAGE. Each formulation was consistent with the A-CF on both the reduced gels and non-reduced gels.

### 1.3. Simulated Shear Stress Study

The formulations at T0 were subjected to simulated shear stress for 24 hours at 900 RPM at room temperature and analysed for osmolality, pH, RALS, DLS, SEC, and SDS-PAGE. The results for osmolality, pH, and RALS are shown in Table 11.

TABLE 11

| | | | | | RALS | | | |
|---|---|---|---|---|---|---|---|---|
| | Osmolality (mOsm/kg) | | pH | | T0 | Shear | Shear | APPEARANCE |
| No. | T0 | Shear | T0 | Shear | (525 Volts) | (525 Volts) | (450 Volts) | Shear |
| A1 | 29 | * | 7.19 | * | 0.917 | 9.999 | 3.215 | clear |
| A2 | 30 | 30 | 7.19 | 7.18 | 1.032 | 9.999 | 3.508 | cloudy |
| A3 | 15 | 20 | 7.82 | * | 1.051 | 8.552 | 1.278 | clear |
| A4 | 15 | 19 | 7.83 | * | 1.124 | 9.999 | 1.654 | clear |
| A5 | 9 | 9 | 6.32 | * | 1.359 | 9.999 | 4.431 | clear |
| A6 | 9 | 12 | 6.33 | * | 1.322 | 9.999 | 4.156 | clear |
| A7 | 2 | 0 | 5.41 | 6.28 | 0.766 | 9.999 | 6.717 | cloudy |
| A8 | −1 | 0 | 5.47 | 6.05 | 1.057 | 9.999 | 6.491 | cloudy |
| A9 | 19 | 17 | 5.82 | 5.88 | 1.22 | 9.999 | 3.022 | slightly turbid |
| A10 | 18 | 19 | 5.81 | 5.95 | 1.254 | 9.999 | 9.275 | cloudy |
| A11 | 40 | 40 | 5.6 | 5.69 | 1.08 | 9.999 | 5.791 | slightly turbid |
| A12 | 41 | 40 | 5.62 | 5.92 | 0.951 | 9.999 | 3.985 | cloudy |
| A13 | 31 | 30 | 5.67 | 5.71 | 1.064 | 9.999 | 5.525 | slightly turbid |
| A14 | 31 | 33 | 5.67 | * | 1.039 | 9.999 | 2.359 | slightly turbid |
| A-CF | 357 | 355 | 7.2 | 7.33 | 0.835 | 9.999 | 5.621 | cloudy |

*Insufficient sample

Consistent with the results obtained for thermal and freeze-thaw stress, formulations at pH higher than pH 7.0 demonstrated relatively less light scattering after simulated shear stress, as shown in Table 11. The data also demonstrate that SONE-IL12-ABD formulations are susceptible to simulated shear stress. All formulations above pH 6.0 remained clear upon simulated shear stress except for formulations containing phosphate without PS20 at pH 7.0.

The formulations were assessed by DLS after incubation after simulated shear stress. As shown in Table 12, all formulations had significant increases in particle size diameter after simulated shear stress, while formulations at pH>7.0, had relatively lower particle size compared to other formulations.

TABLE 12

| | T0 | | | Simulated shear stress | | |
|---|---|---|---|---|---|---|
| No. | PDI | P1 d (nm) | Vol % | PDI | P1 d (nm) | Vol % |
| A1 | 0.503 | 10.51 | 100 | 0.273 | 307.2 | 100 |
| A2 | 0.222 | 10.54 | 100 | 0.599 | 186.0 | 48.4 |
| A3 | 0.295 | 11.61 | 100 | 0.643 | 165.7 | 100 |
| A4 | 0.16 | 9.982 | 100 | 0.598 | 141.4 | 68.9 |
| A5 | 0.148 | 12.32 | 100 | 0.24 | 293.9 | 92.0 |
| A6 | 0.215 | 11.71 | 100 | 0.221 | 300.9 | 100 |
| A7 | 0.226 | 12.16 | 100 | 0.851 | 1322 | 87.2 |
| A8 | 0.203 | 11.48 | 100 | 0.446 | 1067 | 70.6 |
| A9 | 0.267 | 11.53 | 100 | 0.377 | 894.5 | 94.6 |
| A10 | 0.212 | 13.22 | 100 | 0.257 | 617.8 | 73.7 |
| A11 | 0.324 | 11.47 | 100 | 0.238 | 369.5 | 82.7 |
| A12 | 0.213 | 11.81 | 100 | 0.268 | 1168 | 62.6 |
| A13 | 0.299 | 10.37 | 100 | 0.243 | 426.2 | 64.9 |
| A14 | 0.34 | 11.01 | 100 | 0.237 | 392.5 | 100 |
| A-CF | 0.152 | 9.42 | 100 | 0.187 | 482.9 | 79.7 |

The formulations were subjected to simulated shear stress for 24 hours at 900 RPM and monitored by SEC-HPLC for total recovery, fusion protein recovery, and percent impurities consisting of HMW and LMW. The results are shown in Tables 13 and 14. Aggregation was higher in non-PS20 formulations A10 containing acetate and A12 containing citrate. The highest recovery after simulated shear stress was demonstrated in formulations containing Tris.

TABLE 13

| | T0 | | | | | | |
|---|---|---|---|---|---|---|---|
| No. | HMWAUC | MAIN AUC | LMW AUC | TOTAL AUC | % HMW | % MAIN | % LMW |
| A1 | 722 | 70360 | 2954 | 74035 | 0.97 | 95.04 | 3.99 |
| A2 | 801 | 70621 | 2705 | 74127 | 1.08 | 95.27 | 3.65 |
| A3 | 865 | 68561 | 2441 | 71867 | 1.20 | 95.40 | 3.4 |
| A4 | 869 | 68204 | 2272 | 71345 | 1.22 | 95.6 | 3.18 |
| A5 | 1021 | 65934 | 3523 | 70478 | 1.45 | 93.55 | 5.00 |
| A6 | 866 | 67373 | 2245 | 70484 | 1.23 | 95.59 | 3.19 |
| A7 | 1302 | 64671 | 2461 | 68434 | 1.90 | 94.5 | 3.6 |
| A8 | 930 | 63231 | 2696 | 66857 | 1.39 | 94.58 | 4.03 |
| A9 | 1018 | 66465 | 2813 | 70295 | 1.45 | 94.55 | 4.00 |
| A10 | 863 | 69291 | 3032 | 73185 | 1.18 | 94.68 | 4.14 |
| A11 | 945 | 68034 | 3087 | 72066 | 1.31 | 94.41 | 4.28 |
| A12 | 734 | 70844 | 3400 | 74979 | 0.98 | 94.49 | 4.53 |
| A13 | 893 | 67184 | 3340 | 71417 | 1.25 | 94.07 | 4.68 |
| A14 | 757 | 67286 | 3387 | 71430 | 1.06 | 94.2 | 4.74 |
| A-CF | 693 | 70626 | 3138 | 74457 | 0.93 | 94.85 | 4.21 |

TABLE 14

| | Simulated Shear Stress | | | | | | | | |
|---|---|---|---|---|---|---|---|---|---|
| | HMW | MAIN | LMW | TOTAL | % | % | % | % main | % total |
| No. | AUC | AUC | AUC | AUC | HMW | MAIN | LMW | recovery | recovery |
| A1 | 9445 | 37186 | 3266 | 49897 | 18.93 | 74.53 | 6.55 | 52.85 | 67.4 |
| A2 | 6692 | 43831 | 3427 | 53949 | 12.4 | 81.25 | 6.35 | 62.07 | 72.78 |
| A3 | 4592 | 48979 | 5003 | 58574 | 7.84 | 83.62 | 8.54 | 71.44 | 81.5 |
| A4 | 7373 | 46851 | 4474 | 58698 | 12.56 | 79.82 | 7.62 | 68.69 | 82.27 |
| A5 | 1962 | 46038 | 4055 | 52055 | 3.77 | 88.44 | 7.79 | 69.82 | 73.86 |
| A6 | 2546 | 50561 | 4002 | 57109 | 4.46 | 88.53 | 7.01 | 75.05 | 81.02 |
| A7 | 1311 | 25413 | 2251 | 28975 | 4.52 | 87.71 | 7.77 | 39.3 | 42.34 |
| A8 | 1438 | 21746 | 2034 | 25218 | 5.70 | 86.23 | 8.07 | 34.39 | 37.72 |
| A9 | 1496 | 42339 | 3900 | 47736 | 3.13 | 88.7 | 8.17 | 63.7 | 67.91 |
| A10 | 1756 | 18312 | 1237 | 21304 | 8.24 | 85.95 | 5.80 | 26.43 | 29.11 |
| A11 | 781 | 42282 | 2984 | 46047 | 1.70 | 91.82 | 6.48 | 62.15 | 63.90 |
| A12 | 1088 | 18211 | 694 | 19993 | 5.44 | 91.09 | 3.47 | 25.71 | 26.67 |
| A13 | 1394 | 40159 | 2608 | 44161 | 3.16 | 90.94 | 5.91 | 59.77 | 61.84 |
| A14 | 1055 | 53691 | 3384 | 58131 | 1.82 | 92.36 | 5.82 | 79.79 | 81.38 |
| A-CF | 4614 | 10217 | 1445 | 16276 | 28.35 | 62.78 | 8.88 | 14.47 | 21.86 |

The formulations that were subjected to simulated shear stress were also analysed by reducing and non-reducing SDS-PAGE. Each formulation was consistent with the A-CF on reduced gels, except for formulation A12 containing citrate, after shear stress. Non-reduced gels showed more variability and increased degradation in formulation A3 containing Tris with PS20 and HMW streaking in several samples after shear stress.

The results demonstrated that SONE-IL12-ABD formulations containing only buffers with and without PS20 are thermal, freeze-thaw, and shear sensitive. Formulation stability is pH-dependent and formulations at or above pH 7.0 demonstrated relatively more stability when analysed after thermal stress, freeze thaw stress, and simulated shear stress. Formulations containing phosphate and Tris with and without PS20 were relatively more stable compared to other formulations under physical and thermal stress. An example of such a formulation contained 10 mM Tris at pH 7.5 with 0.02% PS20

### Example 2: Stability Studies for Formulations with Varying Ionic Strength

Formulations of SONE-IL12-ABD were prepared as shown in Table 15 using a SONE-IL12-ABD concentration of approximately 0.050 mg/ml.

TABLE 15

| | | Conc. of SONE-IL12- |
|---|---|---|
| No. | Formulation | ABD (mg/ml) |
| B1 | 10 mM glygly, pH 7.5 | 0.039 |
| B2 | 10 mM NaCl, 10 mM glygly, pH 7.5 | 0.044 |
| B3 | 50 mM NaCl, 10 mM glygly, pH 7.5 | 0.047 |
| B4 | 100 mM NaCl, 10 mM glygly, pH 7.5 | 0.038 |
| B5 | 150 mM NaCl, 10 mM glygly, pH 7.5 | 0.021 |
| B6 | 10 mM Tris buffer, pH 7.5 | 0.037 |
| B7 | 10 mM histidine buffer, pH 7.5 | 0.05 |
| B-CF | 20 mM citrate buffer, 10 μM DTPA, | 0.04 |
| | 0.02% PS20, pH 6.2 | |

The formulations were subjected to temperature ramping RALS and IF to find Tagg and Tm of each formulation. Tm and Tagg values are shown in Table 16.

TABLE 16

| No. | Tm (° C.) | Tagg (° C.) |
|---|---|---|
| B1 | 50 | 59 |
| B2 | 49 | 53 |
| B3 | 53 | 56 |
| B4 | 54 | 56 |
| B5 | 54 | 54 |
| B6 | 49 | 59 |
| B7 | 47 | 61 |
| B-CF | 53 | 64 |

Tm was highest for samples with high salt and B-CF. Tagg was the highest for samples with no salt (B1 containing no NaCl, B6 containing Tris, B7 containing histidine, and B-CF).

### 2.1. Thermal Stress Study

The formulations were thermally stressed for 14 days at 40° C. and monitored for osmolality, pH, A280, RALS, Flowcam, SEC-HPLC, and reducing and non-reducing SDS-page. The results for osmolality, pH, A280, and RALS are shown in Tables 17 and 18.

TABLE 17

| | Osmolality | | | | | | RALS | | |
|---|---|---|---|---|---|---|---|---|---|
| | (mOsm/kg) | | | pH | | | (600 Volts) | | |
| No. | T0 | T7 | T14 | T0 | T7 | T14 | T0 | T7 | T14 |
| B1 | 15 | 25 | 14 | 7.73 | 7.85 | 7.91 | 1.081 | 0.854 | 0.979 |
| B2 | 34 | 36 | 33 | 7.66 | 7.81 | 7.87 | 1.108 | 1.06 | 1.014 |
| B3 | 110 | 146 | 111 | 7.64 | 7.81 | 7.83 | 0.964 | 1.06 | 0.834 |
| B4 | 203 | 207 | 202 | 7.63 | 7.75 | 7.80 | 1.018 | 0.866 | 0.929 |
| B5 | 292 | 297 | 292 | 7.63 | 7.78 | 7.80 | 1.060 | 0.910 | 0.866 |
| B6 | 17 | 20 | 21 | 7.61 | 7.74 | 7.75 | 1.310 | 1.023 | 1.155 |
| B7 | 12 | 16 | 14 | 7.63 | 7.91 | 7.96 | 2.513 | 1.072 | 1.550 |
| B-CF | 61 | 63 | 61 | 6.95 | 7.02 | 7.08 | 1.627 | 1.222 | 1.597 |

TABLE 18

| | T0 | | T7 | | T14 | | % Recovery |
|---|---|---|---|---|---|---|---|
| No. | A280 | mg/ml | A280 | mg/ml | A280 | mg/ml | at T14 |
| B1 | 0.06 | 0.039 | 0.058 | 0.038 | 0.052 | 0.034 | 87.1 |
| B2 | 0.067 | 0.044 | 0.059 | 0.039 | 0.052 | 0.034 | 77.3 |
| B3 | 0.072 | 0.047 | 0.061 | 0.04 | 0.044 | 0.029 | 61.7 |
| B4 | 0.058 | 0.038 | 0.06 | 0.039 | 0.051 | 0.033 | 86.8 |
| B5 | 0.032 | 0.021 | 0.065 | 0.042 | 0.045 | 0.029 | 138.1 |
| B6 | 0.056 | 0.037 | 0.074 | 0.048 | 0.073 | 0.048 | 129.7 |
| B7 | 0.077 | 0.050 | 0.071 | 0.046 | 0.078 | 0.051 | 102.0 |
| B-CF | 0.061 | 0.040 | 0.077 | 0.050 | 0.075 | 0.049 | 122.5 |

As shown in Table 18, percent recovery at T14 was low for formulations B2 and B3 containing increasing amounts of salt. Formulation B5 containing high salt had high recovery, as well as formulation B6 containing Tris and B-CF. B1 containing no salt, B2 containing 10 mM NaCl, and B7 containing histidine had recovery between 85% and 105%.

The formulations were assessed for presence of sub-particulates by Flowcam after incubation for 7 and 14 days at 40° C. The results are shown in Table 19.

TABLE 19

| | T0 (P/mL) | | | T7 (P/mL) | | T14 (P/mL) | | |
|---|---|---|---|---|---|---|---|---|
| No. | 2 to 10 μm | 10 to 25 μm | 25 to 50 μm | 2 to 10 μm | 10 to 25 μm | 2 to 10 μm | 10 to 25 μm | 25 to 50 μm |
| B1 | 1899 | 245 | 0 | 500 | 0 | 316 | 64 | 64 |
| B2 | 1072 | 31 | 0 | 75 | 40 | 888 | 276 | 92 |
| B3 | 329 | 110 | 37 | 195 | 40 | 216 | 124 | 32 |
| B4 | 154 | 38 | 0 | 75 | 0 | 216 | 32 | 0 |
| B5 | Insufficient sample | | | 190 | 0 | 536 | 188 | 0 |
| B6 | 245 | 0 | 31 | 115 | 0 | 216 | 60 | 0 |
| B7 | 214 | 31 | 0 | 840 | 0 | 216 | 60 | 32 |
| B-CF | 797 | 153 | 61 | 380 | 0 | 644 | 152 | 60 |

The formulations were thermally stressed for 14 days at 40° C. and monitored by SEC-HPLC for total recovery, fusion protein recovery, and percent impurities consisting of HMW and LMW. The results are shown in Tables 20 to 22. B3 containing 50 mM NaCl had the least amount of % LMW at T14. Formulations containing Tris and histidine (B6 and B7) had a significant increase in HMW and LMW. The formulation containing histidine (B7) had the highest total recovery at T14.

TABLE 20

| | T0 | | | | | | |
|---|---|---|---|---|---|---|---|
| No. | HMWAUC | MAIN AUC | LMW AUC | TOTAL AUC | % HMW | % MAIN | % LMW |
| B1 | 46 | 3172 | 47 | 3265 | 1.4 | 97.2 | 1.4 |
| B2 | 50 | 2970 | 24 | 3044 | 1.6 | 97.6 | 0.8 |
| B3 | 31 | 2923 | 25 | 2979 | 1.1 | 98.1 | 0.8 |
| B4 | 38 | 2977 | 24 | 3039 | 1.2 | 98.0 | 0.8 |
| B5 | 33 | 3124 | 66 | 3223 | 1.0 | 96.9 | 2.0 |
| B6 | 81 | 3621 | 80 | 3782 | 2.2 | 95.7 | 2.1 |
| B7 | 54 | 3449 | 59 | 3562 | 1.5 | 96.8 | 1.7 |
| B-CF | 46 | 3961 | 42 | 4049 | 1.1 | 97.8 | 1.0 |

TABLE 21

| | T7 | | | | | | | |
|---|---|---|---|---|---|---|---|---|
| No. | HMWAUC | MAIN AUC | LMW AUC | TOTAL AUC | % HMW | % MAIN | % LMW | % total recovery |
| B1 | 295 | 1774 | 154 | 2223 | 13.3 | 79.8 | 7.0 | 68.1 |
| B2 | 177 | 1716 | 105 | 1997 | 8.8 | 85.9 | 5.2 | 65.6 |
| B3 | 49 | 1926 | 20 | 1995 | 2.5 | 96.6 | 1.0 | 67.0 |
| B4 | 27 | 1948 | 73 | 2048 | 1.3 | 95.1 | 3.6 | 67.4 |
| B5 | 43 | 2109 | 101 | 2253 | 1.9 | 93.6 | 4.5 | 69.9 |
| B6 | 442 | 2201 | 213 | 2856 | 15.5 | 77.1 | 7.5 | 75.5 |
| B7 | 313 | 2624 | 261 | 3197 | 9.8 | 82.1 | 8.2 | 89.8 |
| B-CF | 59 | 3477 | 176 | 3712 | 1.6 | 93.7 | 4.8 | 91.7 |

TABLE 22

| | T14 | | | | | | | |
|---|---|---|---|---|---|---|---|---|
| No. | HMWAUC | MAIN AUC | LMW AUC | TOTAL AUC | % HMW | % MAIN | % LMW | % total recovery |
| B1 | 483 | 2160 | 92 | 2735 | 17.7 | 79.0 | 3.3 | 83.8 |
| B2 | 378 | 1872 | 73 | 2323 | 16.3 | 80.6 | 3.1 | 76.3 |
| B3 | 110 | 1953 | 39 | 2103 | 5.3 | 92.9 | 1.9 | 70.6 |
| B4 | 48 | 2042 | 45 | 2135 | 2.3 | 95.7 | 2.1 | 70.3 |
| B5 | 34 | 2080 | 54 | 2168 | 1.6 | 95.9 | 2.5 | 67.3 |
| B6 | 811 | 2439 | 130 | 3379 | 24.0 | 72.2 | 3.8 | 89.3 |
| B7 | 470 | 2814 | 124 | 3408 | 13.8 | 82.6 | 3.7 | 95.7 |
| B-CF | 83 | 3564 | 87 | 3734 | 2.2 | 95.4 | 2.3 | 92.2 |

The formulations that were thermally stressed for 14 days at 40° C. were also analyzed by reducing and non-reducing SDS-PAGE. In non-reduced gels, B1 containing no salt, B2 containing 10 mM NaCl, B6 containing Tris, and B7 containing histidine had large HMW bands. B1 containing no salt, B6 containing Tris, B7 containing histidine, and B-CF had higher concentration of LMW bands as well.

### 2.2. Freeze-Thaw Study

The formulations at T0 were subjected to five freeze-thaws and then analyzed for osmolality, pH, RALS, A280, Flowcam, SEC-HPLC, and reducing and non-reducing SDS-page. The results for osmolality, pH, A280, and RALS are shown in Table 23.

TABLE 23

| | Osmolality (mOsm/kg) | | pH | | Concentration | | | | | RALS (600 Volts) | |
|---|---|---|---|---|---|---|---|---|---|---|---|
| | | Freeze- | | Freeze | T0 | | Freeze-thaw | | % | | Freeze- |
| No. | T0 | thaw | T0 | thaw | A280 | mg/ml | A280 | mg/ml | Recovery | T0 | thaw |
| B1 | 15 | 14 | 7.73 | 7.84 | 0.06 | 0.039 | 0.061 | 0.04 | 102.6 | 1.081 | 1.685 |
| B2 | 34 | 33 | 7.66 | 7.81 | 0.067 | 0.044 | 0.056 | 0.037 | 84.10 | 1.108 | 0.888 |
| B3 | 110 | 110 | 7.64 | 7.79 | 0.072 | 0.047 | 0.05 | 0.033 | 70.20 | 0.964 | 0.875 |
| B4 | 203 | 209 | 7.63 | 7.74 | 0.058 | 0.038 | 0.059 | 0.039 | 102.6 | 1.018 | 0.947 |
| B5 | 292 | 312 | 7.63 | 7.67 | 0.032 | 0.021 | 0.057 | 0.037 | 176.2 | 1.06 | 0.894 |
| B6 | 17 | 24 | 7.61 | 7.71 | 0.056 | 0.037 | 0.078 | 0.051 | 137.8 | 1.31 | 3.643 |
| B7 | 12 | 20 | 7.63 | 7.9 | 0.077 | 0.05 | 0.075 | 0.049 | 98.0 | 2.513 | 1.152 |
| B-CF | 61 | 75 | 6.95 | 7.0 | 0.061 | 0.04 | 0.086 | 0.056 | 140.0 | 1.627 | 1.464 |

As shown in Table 23, after freeze-thaw cycling, Formulation B3 containing 50 mM NaCl had low recovery for concentration after freeze-thaw. B5 containing high salt, B6 containing Tris, and B-CF had recoveries above 100%. Further, RALS values after freeze-thaw increased slightly formulation B6 containing Tris.

The formulations were assessed for presence of sub-particulates by Flowcam after five freeze-thaw cycles. The results are shown in Table 24.

TABLE 24

| | T0 FlowCam (P/mL) | | | Freeze-thaw FlowCam (P/mL) | |
|---|---|---|---|---|---|
| | 2 to | 10 to | 25 to | 2 to | 10 to |
| No. | 10 μm | 25 μm | 50 μm | 10 μm | 25 μm |
| B1 | 1899 | 245 | 0 | 216 | 0 |
| B2 | 1072 | 31 | 0 | 216 | 24 |
| B3 | 329 | 110 | 37 | 60 | 0 |
| B4 | 154 | 38 | 0 | 124 | 0 |
| B5 | Insufficient sample | | | 0 | 248 |
| B6 | 245 | 0 | 31 | 92 | 0 |
| B7 | 214 | 31 | 0 | 92 | 0 |
| B-CF | 797 | 153 | 61 | 308 | 0 |

The formulations were subjected to five freeze-thaws and monitored by SEC-HPLC for total recovery, fusion protein recovery, and percent impurities consisting of HMW and LMW. The results are shown in Tables 25 and 26. Formulation B5 containing high salt had no LMW after freeze-thaw. B6 containing Tris, B7 containing histidine, and B-CF all containing no salt had the highest total recoveries after freeze-thaw.

TABLE 25

| | T0 | | | | | | |
|---|---|---|---|---|---|---|---|
| No. | HMWAUC | MAIN AUC | LMW AUC | TOTAL AUC | % HMW | % MAIN | % LMW |
| B1 | 46 | 3172 | 47 | 3265 | 1.4 | 97.2 | 1.4 |
| B2 | 50 | 2970 | 24 | 3044 | 1.6 | 97.6 | 0.8 |
| B3 | 31 | 2923 | 25 | 2979 | 1.1 | 98.1 | 0.8 |
| B4 | 38 | 2977 | 24 | 3039 | 1.2 | 98.0 | 0.8 |
| B5 | 33 | 3124 | 66 | 3223 | 1.0 | 96.9 | 2.0 |
| B6 | 81 | 3621 | 80 | 3782 | 2.2 | 95.7 | 2.1 |
| B7 | 54 | 3449 | 59 | 3562 | 1.5 | 96.8 | 1.7 |
| B-CF | 46 | 3961 | 42 | 4049 | 1.1 | 97.8 | 1.0 |

TABLE 26

| | Freeze-thaw | | | | | | | |
|---|---|---|---|---|---|---|---|---|
| No. | HMW AUC | MAIN AUC | LMW AUC | TOTAL AUC | % HMW | % MAIN | % LMW | % total recovery |
| B1 | 56 | 2337 | 104 | 2497 | 2.2 | 93.6 | 4.2 | 76.5 |
| B2 | 55 | 2096 | 79 | 2231 | 2.5 | 94.0 | 3.6 | 73.3 |
| B3 | 36 | 2155 | 58 | 2249 | 1.6 | 95.8 | 2.6 | 75.5 |
| B4 | 24 | 2093 | 48 | 2165 | 1.1 | 96.7 | 2.2 | 71.2 |
| B5 | 25 | 2297 | 0 | 2322 | 1.1 | 98.9 | 0.0 | 72.0 |
| B6 | 97 | 2898 | 160 | 3155 | 3.1 | 91.9 | 5.1 | 83.4 |
| B7 | 76 | 3038 | 119 | 3233 | 2.4 | 94.0 | 3.7 | 90.8 |
| B-CF | 36 | 3790 | 129 | 3955 | 0.9 | 95.8 | 3.3 | 97.7 |

The formulations that were subjected to five freeze-thaws were also analyzed by reducing and non-reducing SDS-PAGE. No significant differences were observed by SDS-PAGE after five freeze-thaws.

### 2.3. Simulated Shear Stress Study

The formulations at T0 were subjected to simulated shear stress for 24 hours at 900 RPM at room temperature and analysed for osmolality, pH, RALS, DLS, SEC, and SDS-PAGE. The results for osmolality, pH, and RALS are shown in Table 27.

TABLE 27

| | | | | | RALS | |
|---|---|---|---|---|---|---|
| | Osmolality (mOsm/kg) | | pH | | T0 (525 | Shear (525 |
| No. | T0 | Shear | T0 | Shear | Volts) | Volts) |
| B1 | 15 | 14 | 7.73 | 7.79 | 0.229 | 2.144 |
| B2 | 34 | 33 | 7.66 | 7.74 | 0.254 | 2.605 |
| B3 | 110 | 111 | 7.64 | 7.61 | 0.21 | 5.598 |
| B4 | 203 | 203 | 7.63 | 7.68 | 0.233 | 7.637 |
| B5 | 292 | 292 | 7.63 | 7.67 | 0.284 | 3.402 |
| B6 | 17 | 17 | 7.61 | 7.59 | 0.282 | 2.833 |
| B7 | 12 | 13 | 7.63 | 7.7 | 0.559 | 5.434 |
| B-CF | 61 | 62 | 6.95 | 6.95 | 0.379 | 2.245 |

As shown in Table 27, RALS values increased for all formulations, especially for formulations containing 50 mM NaCl, 100 mM NaCl, and histidine (B3, B4, and B7).

The formulations were assessed by DLS after incubation after simulated shear stress. As shown in Table 28, all formulations had large particle size diameters after simulated shear stress. The concentration of the sample was ˜0.050 mg/ml, hence there was no dynamic light scattering observed at this low concentration, however, upon shear stress, the particles increase in size and are therefore measurable by DLS. This was used as a screening tool for simulated shear stressed formulations. The data suggests that formulations containing 0 mM or 10 mM NaCl (D1 and D2) exhibited low light scattering measured by DLS and had a lower particle size relative to other formulations.

TABLE 28

| | Simulated shear stress | | |
|---|---|---|---|
| No. | PDI | P1 d (nm) | Vol % |
| B1 | 0.697 | 88 | 59 |
| B2 | 0.362 | 61 | 64 |
| B3 | 0.297 | 307 | 54 |
| B4 | 0.285 | 73 | 41 |
| B5 | 0.345 | 323 | 100 |
| B6 | 0.609 | 101 | 58 |
| B7 | 0.509 | 132 | 39 |
| B-CF | 0.403 | 137 | 45 |

The formulations were subjected to simulated shear stress for 24 hours at 900 RPM and monitored by SEC-HPLC for total recovery, fusion protein recovery, and percent impurities consisting of HMW and LMW. The results are shown in Tables 29 and 30. Formulation B5 containing high salt and B-CF had the highest % Main after simulated shear stress. Formulation B5 containing high salt and CF also had very low HMW and LMW compared to other formulations. Formulation 1 containing no salt and CF had the highest total percent recoveries above 90%.

TABLE 29

| | T0 | | | | | | |
|---|---|---|---|---|---|---|---|
| No. | HMWAUC | MAIN AUC | LMW AUC | TOTAL AUC | % HMW | % MAIN | % LMW |
| B1 | 46 | 3172 | 47 | 3265 | 1.4 | 97.2 | 1.4 |
| B2 | 50 | 2970 | 24 | 3044 | 1.6 | 97.6 | 0.8 |
| B3 | 31 | 2923 | 25 | 2979 | 1.1 | 98.1 | 0.8 |
| B4 | 38 | 2977 | 24 | 3039 | 1.2 | 98.0 | 0.8 |
| B5 | 33 | 3124 | 66 | 3223 | 1.0 | 96.9 | 2.0 |
| B6 | 81 | 3621 | 80 | 3782 | 2.2 | 95.7 | 2.1 |
| B7 | 54 | 3449 | 59 | 3562 | 1.5 | 96.8 | 1.7 |
| B-CF | 46 | 3961 | 42 | 4049 | 1.1 | 97.8 | 1.0 |

TABLE 30

| | Simulated Shear Stress | | | | | | | |
|---|---|---|---|---|---|---|---|---|
| No. | HMW AUC | MAIN AUC | LMW AUC | TOTAL AUC | % HMW | % MAIN | % LMW | total recovery |
| B1 | 1325 | 1227 | 501 | 3054 | 43.4 | 40.2 | 16.4 | 93.5 |
| B2 | 561 | 1166 | 197 | 1923 | 29.2 | 60.6 | 10.2 | 63.2 |
| B3 | 200 | 101 | 89 | 390 | 51.2 | 26.0 | 22.7 | 13.1 |
| B4 | 120 | 54 | 16 | 189 | 63.3 | 28.5 | 8.3 | 6.2 |
| B5 | 120 | 1228 | 70 | 1418 | 8.5 | 86.6 | 4.9 | 44.0 |
| B6 | 1416 | 809 | 571 | 2797 | 50.6 | 28.9 | 20.4 | 74.0 |
| B7 | 1076 | 1598 | 450 | 3125 | 34.4 | 51.2 | 14.4 | 87.7 |
| B-CF | 188 | 3386 | 347 | 3921 | 4.8 | 86.4 | 8.9 | 96 |

The formulations that were subjected to simulated shear stress were also analysed by reducing and non-reducing SDS-PAGE. Higher aggregation was observed relative to B-CF in the non-reduced gel. Fragmentation was observed in B2 containing 10 mM NaCl and B3 containing 50 mM NaCl.

The results demonstrated that in presence of NaCl SONE-IL12-ABD formulations more susceptible to simulated shear stress. Tm measurements are lower for the lower salt formulations at T0 and Tagg measurements are highest for formulations with no added salt at pH 7.5. The freeze-thaw studies demonstrated similar sensitivity among the formulations, except for B-CF. Further, the temperature stress studies demonstrated that formulations containing no salt had relatively higher total recoveries compared to the formulations containing salt.

### Example 3: Stability Studies for Formulations with Varying Surfactants

Formulations of SONE-IL12-ABD were prepared as shown in Table 31 using a SONE-IL12-ABD concentration of approximately 0.050 mg/ml.

TABLE 31

| No. | Formulation |
|---|---|
| C1 | 25 mM glygly |
| C2 | 25 mM glygly, 0.02% PS20 |
| C3 | 25 mM glygly, 0.05% PS80 |
| C4 | 25 mM glygly, 0.05% Poloxamer 188 |
| C5 | 25 mM glygly, 1.0% PEG 3350 |
| C6 | 25 mM glygly, 0.02% PS20, 10 μM DTPA |
| C-CF | 20 mM citrate, 10 μM DTPA, |
| | 0.02% PS20, pH 6.2 |

The formulations were subjected to temperature ramping RALS and IF to find Tagg and Tm of each formulation. Tm and Tagg values are shown in Table 32.

TABLE 32

| No | Tm (° C.) | Tagg (° C.) |
|---|---|---|
| C1 | 50 | 59 |
| C2 | 50 | 70.5 |
| C3 | 53 | 56.5 |
| C4 | 54.5 | 57.7 |
| C5 | 50 | 60 |
| C6 | 54 | 70 |
| C-CF | 54.5 | 68.5 |

Formulation C6 (0.05 mg/ml SONE-IL12-ABD, 25 mM glygly, 0.02% PS20, 10 μM DTPA) demonstrated high values of both Tm and Tagg, at 54° C. and 70° C. respectively.

### 3.1. Thermal Stress Study

The formulations were thermally stressed for 14 days at 40° C. and monitored for osmolality, pH, A280, RALS, Flowcam, SEC-HPLC, and reducing and non-reducing SDS-page. The results for osmolality, pH, A280, and RALS are shown in Tables 33 and 34.

TABLE 33

| | Osmolality | | | | | | RALS | | |
|---|---|---|---|---|---|---|---|---|---|
| | (mOsm/kg) | | | pH | | | (600 Volts) | | |
| No. | T0 | T7 | T14 | T0 | T7 | T14 | T0 | T7 | T14 |
| C1 | 36 | 33 | 37 | 7.53 | 7.53 | 7.61 | 0.93 | 0.825 | 1.072 |
| C2 | 33 | 30 | 34 | 7.53 | 7.55 | 7.59 | 1.09 | 1.566 | 1.022 |
| C3 | 33 | 31 | 34 | 7.53 | 7.55 | 7.59 | 2.9 | 2.492 | 2.223 |
| C4 | 33 | 31 | 35 | 7.52 | 7.56 | 7.60 | 1.09 | 1.195 | 1.016 |
| C5 | 41 | 38 | 41 | 7.54 | 7.59 | 7.61 | 2.43 | 1.726 | 2.250 |
| C6 | 33 | 30 | 38 | 7.54 | 7.57 | 7.61 | 1.54 | 1.239 | 1.198 |
| C-CF | 62 | 61 | 63 | 7.01 | 7.07 | 7.06 | 1.51 | 1.291 | 1.153 |

TABLE 34

| | T0 | | T7 | | T14 | | % Recovery |
|---|---|---|---|---|---|---|---|
| No. | A280 | mg/ml | A280 | mg/ml | A280 | mg/ml | at T14 |
| C1 | 0.070 | 0.046 | 0.055 | 0.036 | 0.044 | 0.029 | 63.0 |
| C2 | 0.079 | 0.052 | 0.066 | 0.043 | 0.059 | 0.039 | 75.0 |
| C3 | 0.077 | 0.05 | 0.066 | 0.043 | 0.047 | 0.031 | 62.0 |
| C4 | 0.08 | 0.052 | 0.068 | 0.044 | 0.063 | 0.041 | 78.8 |
| C5 | 0.067 | 0.044 | 0.06 | 0.039 | 0.061 | 0.04 | 90.9 |
| C6 | 0.071 | 0.046 | 0.068 | 0.044 | 0.051 | 0.033 | 71.7 |
| C-CF | 0.07 | 0.046 | 0.069 | 0.045 | 0.062 | 0.04 | 87.0 |

As shown in Table 33, there was a slight increase in pH during the 14-day incubation. Formulation C5 containing PEG 3350 and C-CF had the highest percent recovery in concentration by A280 at T14 of around 90%.

The formulations were assessed for presence of sub-particulates by Flowcam after incubation for 7 and 14 days at 40° C. As shown in Table 35, sub-visible particle counts increased for each formulation at T7 and T14, and particles increased the most for C4 containing poloxamer 188.

TABLE 35

| | 2 to 10 | 10 to 25 | 25 to 50 | 50 to 100 | 100+ |
|---|---|---|---|---|---|
| No. | μm | μm | μm | μm | μm |
| | T0 FlowCam (P/mL) | | | | |
| C1 | 92 | 31 | 0 | 0 | 0 |
| C2 | 124 | 31 | 31 | 0 | 0 |
| C3 | 736 | 184 | 60 | 0 | 31 |
| C4 | 644 | 38 | 8 | 0 | 0 |
| C5 | 92 | 60 | 0 | 0 | 0 |
| C6 | 124 | 31 | 0 | 0 | 0 |
| C-CF | 184 | 92 | 31 | 0 | 0 |
| | T7 FlowCam (P/mL) | | | | |
| C1 | 668 | 180 | 88 | 0 | 0 |
| C2 | 492 | 92 | 0 | 31 | 31 |
| C3 | 888 | 216 | 92 | 0 | 0 |
| C4 | 400 | 92 | 31 | 0 | 0 |
| C5 | 764 | 31 | 0 | 0 | 0 |
| C6 | 704 | 0 | 0 | 0 | 0 |
| C-CF | 552 | 124 | 60 | 0 | 0 |
| | T14 FlowCam (P/mL) | | | | |
| C1 | 460 | 60 | 31 | 31 | 0 |
| C2 | 492 | 92 | 31 | 0 | 0 |
| C3 | 368 | 0 | 31 | 0 | 0 |
| C4 | 2452 | 552 | 60 | 0 | 0 |
| C5 | 1012 | 31 | 0 | 0 | 0 |
| C6 | 1592 | 92 | 0 | 0 | 0 |
| C-CF | 764 | 92 | 60 | 0 | 0 |

The formulations were thermally stressed for 14 days at 40° C. and monitored by SEC-HPLC for total recovery, fusion protein recovery, and percent impurities consisting of HMW and LMW. The results are shown in Tables 36 to 38. After 14 days of incubation, all formulations except C-CF demonstrated a large increase in % HMW. The total recoveries at T14 ranged from 88% for formulation C2 (containing 25 mM glygly, 0.02% PS20) to 96% for formulation C6 (containing 25 mM glygly, 0.02% PS20, and 10 μM DTPA). C-CF had a percent recovery of 103%.

TABLE 36

| | T0 | | | | | | |
|---|---|---|---|---|---|---|---|
| No. | % HMW | % Main | % LMW | HMW AUG | Main AUC | LMW AUC | Total AUC |
| C1 | 1.4 | 97.3 | 1.3 | 70 | 4799 | 62 | 4931 |
| C2 | 0.6 | 98.0 | 1.4 | 33 | 5411 | 76 | 5520 |
| C3 | 0.9 | 95.5 | 3.6 | 48 | 5082 | 189 | 5320 |
| C4 | 0.8 | 93.4 | 5.8 | 41 | 4995 | 312 | 5348 |
| C5 | 0.7 | 95.8 | 3.5 | 34 | 4616 | 170 | 4820 |
| C6 | 0.6 | 98.5 | 0.9 | 28 | 4733 | 45 | 4806 |
| C-CF | 0.3 | 99.1 | 0.6 | 17 | 5427 | 32 | 5476 |

TABLE 37

| | T7 | | | | | | | |
|---|---|---|---|---|---|---|---|---|
| No. | % HMW | % Main | % LMW | HMW AUC | Main AUC | LMW AUC | Total AUC | % total recovery |
| C1 | 7.3 | 88.7 | 4.1 | 293 | 3573 | 165 | 4031 | 81.7 |
| C2 | 8.1 | 86.0 | 6.0 | 389 | 4138 | 287 | 4814 | 87.2 |
| C3 | 5.8 | 90.5 | 3.7 | 273 | 4253 | 174 | 4701 | 88.4 |
| C4 | 5.9 | 90.5 | 3.6 | 300 | 4612 | 184 | 5096 | 95.3 |
| C5 | 8.1 | 86.7 | 5.1 | 371 | 3947 | 234 | 4552 | 94.4 |
| C6 | 5.8 | 91.6 | 2.6 | 272 | 4286 | 122 | 4680 | 97.4 |
| C-CF | 0.9 | 98.0 | 1.1 | 50 | 5164 | 57 | 5271 | 96.3 |

TABLE 38

| | T14 | | | | | | | |
|---|---|---|---|---|---|---|---|---|
| No. | % HMW | % Main | % LMW | HMW AUC | Main AUC | LMW AUC | Total AUC | % Total recovery |
| C1 | 12.5 | 83.5 | 4.0 | 551 | 3696 | 177 | 4424 | 89.7 |
| C2 | 10.5 | 88.6 | 0.9 | 511 | 4320 | 43 | 4874 | 88.3 |
| C3 | 7.6 | 91.2 | 1.2 | 374 | 4480 | 59 | 4913 | 92.3 |
| C4 | 11.1 | 87.4 | 1.6 | 564 | 4454 | 81 | 5098 | 95.3 |
| C5 | 12.5 | 86.8 | 0.7 | 550 | 3805 | 30 | 4385 | 91.0 |
| C6 | 12.0 | 86.8 | 1.2 | 559 | 4025 | 56 | 4640 | 96.5 |
| C-CF | 2.4 | 94.2 | 3.4 | 136 | 5338 | 194 | 5668 | 103.5 |

The formulations that were thermally stressed for 14 days at 40° C. were also analyzed by reducing and non-reducing SDS-PAGE. T0 samples demonstrated no HMW in non-reduced gels, and non-reduced gels showed more variability with increased degradation. C6 (containing PS20 and DTPA) demonstrated 100% main in non-reduced gels at T14.

### 3.2. Freeze-Thaw Study

The formulations at T0 were subjected to five freeze-thaws and then analyzed for osmolality, pH, RALS, A280, Flowcam, SEC-HPLC, and reducing and non-reducing SDS-page. The results for osmolality, pH, A280, and RALS are shown in Table 39.

TABLE 39

| | Osmolality (mOsm/kg) | | pH | | Concentration | | | | | RALS (600 Volts) | |
|---|---|---|---|---|---|---|---|---|---|---|---|
| | | Freeze- | | Freeze- | T0 | | Freeze-thaw | | % | | Freeze- |
| No. | T0 | thaw | T0 | thaw | A280 | mg/ml | A280 | mg/ml | Recovery | T0 | thaw |
| C1 | 36 | 32 | 7.53 | 7.56 | 0.07 | 0.046 | 0.058 | 0.038 | 82.3 | 0.93 | 0.82 |
| C2 | 33 | 32 | 7.53 | 7.55 | 0.079 | 0.052 | 0.081 | 0.053 | 101.7 | 1.09 | 1.07 |
| C3 | 33 | 32 | 7.53 | 7.56 | 0.077 | 0.05 | 0.065 | 0.042 | 84.9 | 2.9 | 2.48 |
| C4 | 33 | 32 | 7.52 | 7.55 | 0.08 | 0.052 | 0.07 | 0.046 | 87.9 | 1.09 | 1.04 |
| C5 | 41 | 38 | 7.54 | 7.56 | 0.067 | 0.044 | 0.063 | 0.041 | 93.5 | 2.43 | 1.77 |
| C6 | 33 | 32 | 7.54 | 7.56 | 0.071 | 0.046 | 0.067 | 0.044 | 95.1 | 1.54 | 1.16 |
| C-CF | 62 | 60 | 7.01 | 7.04 | 0.07 | 0.046 | 0.066 | 0.043 | 93.7 | 1.51 | 1.1 |

As shown in Table 39, there was a slight decrease in concentration after freeze-thaw except for C2 (25 mM glygly, 0.02% PS20), at 101% recovery.

The formulations were assessed for presence of sub-particulates by Flowcam after five freeze-thaw cycles. The results are shown in Table 40. Sub-visible particles increased for each formulation after freeze-thaw. Particles increased the most for C6 (25 mM glygly, 0.02% PS20, 10 μM DTPA).

TABLE 40

| | 2 to 10 | 10 to 25 | 25 to 50 | 50 to 100 | 100+ |
|---|---|---|---|---|---|
| No. | μm | μm | μm | μm | μm |
| | T0 FlowCam (P/mL) | | | | |
| C1 | 92 | 31 | 0 | 0 | 0 |
| C2 | 124 | 31 | 31 | 0 | 0 |
| C3 | 736 | 184 | 60 | 0 | 31 |
| C4 | 644 | 38 | 8 | 0 | 0 |
| C5 | 92 | 60 | 0 | 0 | 0 |
| C6 | 124 | 31 | 0 | 0 | 0 |
| C-CF | 184 | 92 | 31 | 0 | 0 |
| | Freeze-thaw FlowCam (P/mL) | | | | |
| C1 | 368 | 31 | 31 | 0 | 0 |
| C2 | 2052 | 552 | 400 | 0 | 0 |
| C3 | 1348 | 152 | 0 | 31 | 0 |
| C4 | 644 | 216 | 0 | 60 | 0 |
| C5 | 1072 | 60 | 0 | 0 | 0 |
| C6 | 2848 | 644 | 124 | 31 | 0 |
| C-CF | 492 | 92 | 31 | 0 | 0 |

The formulations were subjected to five freeze-thaws and monitored by SEC-HPLC for total recovery, fusion protein recovery, and percent impurities consisting of HMW and LMW. The results are shown in Tables 41 and 42. C3 containing PS80, C6 containing PS20 and DTPA, and C-CF had 100% recovery after freeze-thaw on SEC. There was a slight increase in HMW in all formulations except C1 containing glygly. Each formulation had a percent main peak of above 94%. C1 containing glygly had the least amount of LMW after freeze-thaw.

TABLE 41

| | T0 | | | | | | |
|---|---|---|---|---|---|---|---|
| | % | % | % | HMW | Main | LMW | Total |
| No. | HMW | Main | LMW | AUC | AUC | AUC | AUC |
| C1 | 1.4 | 97.3 | 1.3 | 70 | 4799 | 62 | 4931 |
| C2 | 0.6 | 98.0 | 1.4 | 33 | 5411 | 76 | 5520 |
| C3 | 0.9 | 95.5 | 3.6 | 48 | 5082 | 189 | 5320 |
| C4 | 0.8 | 93.4 | 5.8 | 41 | 4995 | 312 | 5348 |
| C5 | 0.7 | 95.8 | 3.5 | 34 | 4616 | 170 | 4820 |
| C6 | 0.6 | 98.5 | 0.9 | 28 | 4733 | 45 | 4806 |
| C-CF | 0.3 | 99.1 | 0.6 | 17 | 5427 | 32 | 5476 |

TABLE 42

| | Freeze-thaw | | | | | | | |
|---|---|---|---|---|---|---|---|---|
| No. | % HMW | % Main | % LMW | HMW AUC | Main AUC | LMW AUC | Total AUC | % total recovery |
| C1 | 1.0 | 98.8 | 0.2 | 39 | 4012 | 10 | 4061 | 82.4 |
| C2 | 0.9 | 97.5 | 1.6 | 46 | 5213 | 86 | 5345 | 96.8 |
| C3 | 1.7 | 95.6 | 2.7 | 92 | 5100 | 142 | 5334 | 100.3 |
| C4 | 1.2 | 94.4 | 4.4 | 57 | 4574 | 212 | 4843 | 90.6 |
| C5 | 1.5 | 97.6 | 0.9 | 64 | 4017 | 36 | 4117 | 85.4 |
| C6 | 2.1 | 95.4 | 2.5 | 103 | 4647 | 122 | 4872 | 101.4 |
| C-CF | 1.8 | 96.5 | 1.7 | 104 | 5536 | 96 | 5737 | 104.8 |

The formulations that were subjected to five freeze-thaws were also analyzed by reducing and non-reducing SDS-PAGE. % LMW increased slightly for each formulation on reduced gels after freeze-thaw. There was no change seen in non-reduced gels and showed 100% main peak.

### 3.3. Simulated Shear Stress Study

The formulations at T0 were subjected to simulated shear stress for 24 hours at 900 RPM at room temperature and analysed for osmolality, pH, A280, RALS, SEC, and SDS-PAGE. The results for osmolality, pH, A280, and RALS are shown in Table 43.

TABLE 43

| | | | | | Concentration | | | | | | APPEARANCE |
|---|---|---|---|---|---|---|---|---|---|---|---|
| | Osmolality (mOsm/kg) | | pH | | T0 | | Shear | | RALS (600 Volts) | | after simulated |
| No. | T0 | Shear | T0 | Shear | A280 | mg/ml | A280 | mg/ml | T0 | Shear | shear |
| C1 | 36 | 33 | 7.53 | 7.55 | 0.07 | 0.046 | 0.066 | 0.043 | 0.93 | 8.57 | Clear |
| C2 | 33 | 32 | 7.53 | 7.56 | 0.079 | 0.052 | 0.064 | 0.042 | 1.09 | 4.70 | Cloudy |
| C3 | 33 | 34 | 7.53 | 7.55 | 0.077 | 0.05 | 0.076 | 0.05 | 2.9 | 8.12 | Cloudy |
| C4 | 33 | 32 | 7.52 | 7.54 | 0.08 | 0.052 | 0.068 | 0.044 | 1.09 | 4.64 | Cloudy |
| C5 | 41 | 40 | 7.54 | 7.56 | 0.067 | 0.044 | 0.073 | 0.048 | 2.43 | 5.86 | Clear |
| C6 | 33 | 33 | 7.54 | 7.54 | 0.071 | 0.046 | 0.066 | 0.043 | 1.54 | 3.75 | Clear |
| C-CF | 62 | 60 | 7.01 | 7.01 | 0.07 | 0.046 | 0.078 | 0.051 | 1.51 | 9.99 | Clear |

As shown in Table 43, RALS values increased significantly for the formulation containing glygly (C1), the formulation containing PS80 (C3), and C-CF after simulated shear stress indicating these formulations are relatively more sensitive to shear stress. C6 containing 25 mM glygly, 0.02% PS20, 10 μM DPTA had the lowest RALS value indicating that this formulation is relatively less sensitive to shear stress. Formulations containing PS20, PS80 and poloxamer (C2, C3, and C4), respectively, had a cloudy appearance after simulated shear stress.

The formulations were subjected to simulated shear stress for 24 hours at 900 RPM and monitored by SEC-HPLC for total recovery, fusion protein recovery, and percent impurities consisting of HMW and LMW. The results are shown in Tables 44 and 45. The results demonstrated that C6 (25 mM glygly, 0.02% PS20, 10 μM DTPA) had relatively higher total area recovery compared to the other formulations.

TABLE 44

| | T0 | | | | | | |
|---|---|---|---|---|---|---|---|
| | % | % | % | HMW | Main | LMW | Total |
| No. | HMW | Main | LMW | AUC | AUC | AUC | AUC |
| C1 | 1.4 | 97.3 | 1.3 | 70 | 4799 | 62 | 4931 |
| C2 | 0.6 | 98.0 | 1.4 | 33 | 5411 | 76 | 5520 |
| C3 | 0.9 | 95.5 | 3.6 | 48 | 5082 | 189 | 5320 |
| C4 | 0.8 | 93.4 | 5.8 | 41 | 4995 | 312 | 5348 |
| C5 | 0.7 | 95.8 | 3.5 | 34 | 4616 | 170 | 4820 |
| C6 | 0.6 | 98.5 | 0.9 | 28 | 4733 | 45 | 4806 |
| C-CF | 0.3 | 99.1 | 0.6 | 17 | 5427 | 32 | 5476 |

TABLE 45

| | Simulated Shear Stress | | | | | | | |
|---|---|---|---|---|---|---|---|---|
| No. | % HMW | % Main | % LMW | HMW AUC | Main AUC | LMW AUC | Total AUC | % total recovery |
| C1 | 38.4 | 58.6 | 3.0 | 765 | 1166 | 60 | 1991 | 40.4 |
| C2 | 33.0 | 64.9 | 2.1 | 1160 | 2283 | 73 | 3516 | 63.7 |
| C3 | 47.5 | 50.9 | 1.7 | 1507 | 1616 | 52 | 3176 | 59.7 |
| C4 | 30.8 | 66.2 | 3.0 | 1121 | 2406 | 109 | 3636 | 68.0 |
| C5 | 67.8 | 29.7 | 2.6 | 2035 | 892 | 77 | 3004 | 62.3 |
| C6 | 14.5 | 80.8 | 4.7 | 547 | 3046 | 179 | 3772 | 78.5 |
| C-CF | 56.7 | 40.9 | 2.4 | 1601 | 1153 | 68 | 2823 | 51.6 |

The formulations that were subjected to simulated shear stress were also analysed by reducing and non-reducing SDS-PAGE. Reduced gels did not show any significant differences after shear, but non-reduced gels showed an increase in HMW. C6 (25 mM glygly, 0.02% PS20, 10 μM DTPA) had the least amount of HMW on the gels after shear.

The SDS-PAGE and SEC data after thermal stress, and the RALS and SDS-PAGE data after simulated shear stress, demonstrated that PS20 improves stability. The SEC total recovery data after thermal stress demonstrated that C6 (containing PS20 and DTPA) and C-CF are relatively more stable among the formulations. Further, testing after freeze-thaw stress demonstrated that C2 (containing PS20, C3 containing PS80, C6 containing PS20 and DTPA, and C-CF) were relatively more stable against freeze-thaw stress compared to other formulations.

### Example 4: Stability Studies for Formulations with Varying Amino Acid Stabilizers

Formulations of SONE-IL12-ABD were prepared as shown in Table 46 using a SONE-IL12-ABD concentration of approximately 20 μg/ml.

TABLE 46

| No. | Formulation |
|---|---|
| D-CF | 25 mM GlyGly, 10 μM DTPA, 0.02% PS20 |
| D1 | 10 mM aspartic acid, 25 mM GlyGly, 10 μM DTPA, 0.02% PS20 |
| D2 | 50 mM serine, 25 mM GlyGly, 10 μM DTPA, 0.02% PS20 |
| D3 | 50 mM alanine, 25 mM GlyGly, 10 μM DTPA, 0.02% PS20 |
| D4 | 50 mM glycine, 25 mM GlyGly, 10 μM DTPA, 0.02% PS20 |
| D5 | 50 mM arginine, 25 mM GlyGly, 10 μM DTPA, 0.02% PS20 |
| D6 | 50 mM lysine, 25 mM GlyGly, 10 μM DTPA, 0.02% PS20 |
| D7 | 50 mM proline, 25 mM GlyGly, 10 μM DTPA, 0.02% PS20 |

The formulations were subjected to temperature ramping RALS and IF to find Tagg and Tm of each formulation. Tm and Tagg values are shown in Table 46A.

TABLE 46A

| No | Tm (° C.) | Tagg (° C.) |
|---|---|---|
| D-CF | 46 | 68 |
| D1 | 46 | 69 |
| D2 | 47 | 69 |
| D3 | 47 | 71 |
| D4 | 46 | 68 |
| D5 | 48 | 69 |
| D6 | 48 | 69 |
| D7 | 48 | 71 |

### 4.1. Thermal Stress Study

The formulations were thermally stressed for 14 days at 40° C. and monitored for osmolality, pH, A280, RALS, Flowcam, SEC-HPLC, and reducing and non-reducing SDS-page. The results for osmolality, pH, A280, and RALS are shown in Tables 47 and 48.

TABLE 47

| | Osmolality | | | | | | RALS | | |
|---|---|---|---|---|---|---|---|---|---|
| | (mOsm/kg) | | | pH | | | (565 Volts) | | |
| No. | T0 | T7 | T14 | T0 | T7 | T14 | T0 | T7 | T14 |
| D-CF | 30 | 34 | 31 | 7.52 | 7.51 | 7.49 | 1.08 | 0.93 | 0.91 |
| D1 | 44 | 46 | 44 | 7.53 | 7.52 | 7.58 | 1.12 | 0.99 | 0.86 |
| D2 | 83 | 87 | 83 | 7.48 | 7.46 | 7.52 | 1.00 | 1.07 | 1.28 |
| D3 | 78 | 80 | 76 | 7.55 | 7.59 | 7.6 | 1.13 | 1.31 | 0.89 |
| D4 | 78 | 78 | 78 | 7.51 | 7.62 | 7.54 | 1.26 | 0.93 | 0.90 |
| D5 | 121 | 121 | 122 | 7.51 | 7.47 | 7.46 | 1.08 | 0.94 | 0.92 |
| D6 | 122 | 123 | 124 | 7.53 | 7.54 | 7.48 | 1.07 | 0.99 | 0.89 |
| D7 | 80 | 82 | 80 | 7.52 | 7.58 | 7.51 | 1.25 | 0.91 | 1.01 |

TABLE 48

| | | | | | % | | | % |
|---|---|---|---|---|---|---|---|---|
| | T0 | | T7 | | Recovery | T14 | | Recovery |
| No. | A280 | μg/ml | A280 | μg/ml | at T7 | A280 | μg/ml | at T14 |
| D-CF | 0.033 | 21.54 | 0.025 | 16.32 | 75.8 | 0.023 | 15.01 | 69.7 |
| D1 | 0.034 | 22.19 | 0.026 | 16.97 | 76.5 | 0.025 | 16.32 | 73.6 |
| D2 | 0.029 | 18.93 | 0.026 | 16.97 | 89.7 | 0.031 | 20.23 | 106.9 |
| D3 | 0.033 | 21.54 | 0.026 | 16.97 | 78.8 | 0.026 | 16.97 | 78.8 |
| D4 | 0.029 | 18.93 | 0.026 | 16.97 | 89.7 | 0.024 | 15.67 | 84.4 |
| D5 | 0.035 | 22.85 | 0.025 | 16.32 | 71.4 | 0.027 | 17.62 | 77.1 |
| D6 | 0.032 | 20.89 | 0.022 | 14.36 | 68.7 | 0.029 | 18.93 | 90.6 |
| D7 | 0.03 | 19.58 | 0.022 | 14.36 | 73.3 | 0.026 | 16.97 | 86.7 |

As shown in Table 48, concentration decreased for each formulation after 14 days of incubation except for formulation D2 (containing serine).

The formulations were assessed for presence of sub-particulates by Flowcam after incubation for 7 and 14 days at 40° C. As shown in Table 49, sub-visible particle counts at T14 increased for most formulations, with the exception of formulation D5 (containing arginine). Sub-visible particle counts at T14 for formulation D7 (containing proline) increased significantly.

TABLE 49

| | | | | |
|---|---|---|---|---|
| No. | 2-10 um | 10-25 um | 25-50 um | 50-100 um |

T0

| | | | | |
|---|---|---|---|---|
| D-CF | 612 | 124 | 0 | 0 |
| D1 | 428 | 184 | 152 | 0 |
| D2 | 460 | 152 | 92 | 31 |
| D3 | 400 | 216 | 60 | 0 |
| D4 | 244 | 60 | 0 | 31 |
| D5 | 520 | 184 | 60 | 0 |
| D6 | 980 | 552 | 276 | 31 |
| D7 | 948 | 244 | 60 | 0 |

T7

| | | | | |
|---|---|---|---|---|
| D-CF | 500 | 124 | 31 | 0 |
| D1 | 336 | 31 | 0 | 0 |
| D2 | 1980 | 588 | 184 | 0 |
| D3 | 672 | 244 | 31 | 0 |
| D4 | 428 | 124 | 31 | 0 |
| D5 | 1308 | 312 | 124 | 0 |
| D6 | 1132 | 368 | 92 | 92 |
| D7 | 1776 | 308 | 31 | 60 |

T14

| | | | | |
|---|---|---|---|---|
| D-CF | 1808 | 308 | 31 | 0 |
| D1 | 2480 | 492 | 0 | 31 |
| D2 | 4500 | 1608 | 128 | 0 |
| D3 | 1304 | 152 | 116 | 0 |
| D4 | 1392 | 308 | 60 | 0 |
| D5 | 1012 | 244 | 140 | 0 |
| D6 | 3236 | 468 | 31 | 31 |
| D7 | 21964 | 380 | 0 | 0 |

The formulations were thermally stressed for 14 days at 40° C. and monitored by SEC-HPLC for total recovery, fusion protein recovery, and percent impurities consisting of HMW and LMW. The results are shown in Tables 50 to 52. Formulations D5 (containing arginine) and D6 (containing lysine) had the highest percent main peak on SEC. HMW increased at T7 and T14, with the lowers increases demonstrated for D5 (containing arginine) and D6 (containing lysine).

TABLE 50

| | T0 | | | | | | |
|---|---|---|---|---|---|---|---|
| | % | % | % | HMW | Main | LMW | Total |
| No. | HMW | Main | LMW | AUC | AUC | AUC | AUC |
| D-CF | 1.2 | 71.9 | 26.9 | 134 | 7787 | 2910 | 10832 |
| D1 | 1.3 | 90.3 | 8.4 | 117 | 8203 | 761 | 9081 |
| D2 | 1.2 | 95.3 | 3.5 | 93 | 7543 | 275 | 7912 |
| D3 | 1.4 | 92.5 | 6.1 | 117 | 7569 | 498 | 8185 |
| D4 | 1.5 | 95.6 | 2.9 | 124 | 7837 | 237 | 8198 |
| D5 | 0.6 | 96.5 | 2.9 | 49 | 7758 | 236 | 8043 |
| D6 | 0.6 | 97.2 | 2.2 | 46 | 8050 | 182 | 8278 |
| D7 | 3.9 | 93.1 | 3.0 | 321 | 7625 | 241 | 8189 |

TABLE 51

| | T7 | | | | | | | |
|---|---|---|---|---|---|---|---|---|
| No. | % HMW | % Main | % LMW | HMW AUC | Main AUC | LMW AUC | Total AUC | % total recovery |
| D-CF | 16.1 | 77.0 | 6.9 | 1175 | 5613 | 506 | 7294 | 67.3 |
| D1 | 10.6 | 86.2 | 3.2 | 747 | 6103 | 225 | 7076 | 77.9 |
| D2 | 16.4 | 79.1 | 4.5 | 1188 | 5748 | 326 | 7263 | 91.8 |
| D3 | 13.4 | 84.2 | 2.4 | 936 | 5887 | 170 | 6995 | 85.5 |
| D4 | 14.5 | 84.0 | 1.5 | 986 | 5724 | 103 | 6815 | 83.1 |
| D5 | 1.5 | 95.3 | 3.2 | 108 | 6995 | 236 | 7340 | 91.3 |
| D6 | 1.0 | 96.5 | 2.5 | 82 | 7693 | 196 | 7972 | 96.3 |
| D7 | 12.6 | 85.6 | 1.7 | 886 | 6019 | 122 | 7028 | 85.8 |

TABLE 52

| | T14 | | | | | | | |
|---|---|---|---|---|---|---|---|---|
| No. | % HMW | % Main | % LMW | HMW AUC | Main AUC | LMW AUC | Total AUC | % Total recovery |
| D-CF | 18.8 | 66.8 | 14.5 | 1634 | 5811 | 1255 | 8704 | 80.4 |
| D1 | 14.0 | 80.1 | 5.9 | 1145 | 6562 | 482 | 8192 | 90.2 |
| D2 | 25.6 | 69.4 | 5.0 | 1955 | 5311 | 381 | 7651 | 96.7 |
| D3 | 16.5 | 78.6 | 4.8 | 1267 | 6033 | 368 | 7672 | 93.7 |
| D4 | 19.1 | 76.2 | 4.6 | 1487 | 5929 | 358 | 7777 | 94.9 |
| D5 | 1.7 | 92.9 | 5.4 | 137 | 7653 | 444 | 8236 | 102.4 |
| D6 | 2.0 | 93.0 | 5.0 | 165 | 7828 | 422 | 8417 | 101.7 |
| D7 | 21.0 | 74.7 | 4.3 | 1690 | 6021 | 347 | 8061 | 98.4 |

The formulations that were thermally stressed for 14 days at 40° C. were also analyzed by reducing and non-reducing SDS-PAGE. The results are shown in Tables 53 and 54. T0 samples demonstrated 100% main band with no HMW or LMW. Each formulation behaved similarly at T7 and T14. Some HMW was seen in reduced gels, whereas some LMW was present in non-reduced gels.

TABLE 53

| | |
|---|---|
| T7 Non-Reduced | T7 Reduced |

| No | % Main | % HMW | No. | % Main | % LMW |
|---|---|---|---|---|---|
| D-CF | 99.06 | 0.94 | D-CF | 97.21 | 2.79 |
| D1 | 99.35 | 0.65 | D1 | 100 | 0 |
| D2 | 100 | 0 | D2 | 97.08 | 2.92 |
| D3 | 99.16 | 0.84 | D3 | 99.02 | 0.98 |
| D4 | 99.8 | 0.2 | D4 | 99.3 | 0.7 |
| D5 | 100 | 0 | D5 | 99.05 | 0.95 |
| D6 | 100 | 0 | DE | 98.72 | 1.28 |
| D7 | 100 | 0 | D7 | 99.08 | 0.92 |

TABLE 54

| | |
|---|---|
| T14 Non-Reduced | T14 Reduced |

| No. | % Main | % HMW | No | % Main | % LMW |
|---|---|---|---|---|---|
| D-CF | 98.88 | 1.12 | D-CF | 92.02 | 7.98 |
| D1 | 99.44 | 0.56 | D1 | 98.66 | 1.34 |
| D2 | 97.73 | 2.27 | D2 | 90.72 | 9.28 |
| D3 | 99.33 | 0.67 | D3 | 99 | 1 |
| D4 | 99.19 | 0.81 | D4 | 99.21 | 0.79 |
| D5 | 100 | 0 | D5 | 98.98 | 1.02 |
| D6 | 100 | 0 | D6 | 99.22 | 0.78 |
| D7 | 99.15 | 0.85 | D7 | 99.83 | 0.17 |

### 4.2. Freeze-Thaw Study

The formulations at T0 were subjected to five freeze-thaws and then analyzed for osmolality, pH, RALS, A280, Flowcam, SEC-HPLC, and reducing and non-reducing SDS-page. The results for osmolality, pH, A280, and RALS are shown in Table 55.

TABLE 55

| | Osmolality (mOsm/kg) | | pH | | Concentration | | | | RALS (565 Volts) | |
|---|---|---|---|---|---|---|---|---|---|---|
| | | Freeze- | | Freeze- | T0 | | Freeze-thaw | | | Freeze- |
| No. | T0 | thaw | T0 | thaw | A280 | μg/ml | A280 | μg/ml | T0 | thaw |
| D-CF | 30 | 30 | 7.52 | 7.55 | 0.033 | 21.54 | 0.028 | 18.28 | 1.08 | 0.957 |
| D1 | 44 | 44 | 7.53 | 7.54 | 0.034 | 22.19 | 0.03 | 19.58 | 1.12 | 0.968 |
| D2 | 83 | 82 | 7.48 | 7.51 | 0.029 | 18.93 | 0.03 | 19.58 | 1.00 | 0.955 |
| D3 | 78 | 77 | 7.55 | 7.56 | 0.033 | 21.54 | 0.028 | 18.28 | 1.13 | 0.931 |
| D4 | 78 | 78 | 7.51 | 7.52 | 0.029 | 18.93 | 0.023 | 15.01 | 1.26 | 1.086 |
| D5 | 121 | 121 | 7.51 | 7.5 | 0.035 | 22.85 | 0.032 | 20.89 | 1.08 | 1.005 |
| D6 | 122 | 122 | 7.53 | 7.5 | 0.032 | 20.89 | 0.026 | 16.97 | 1.07 | 0.943 |
| D7 | 80 | 81 | 7.52 | 7.51 | 0.03 | 19.58 | 0.057 | 37.21 | 1.25 | 1.915 |

As shown in Table 55, concentration decreased slightly after freeze-thaw, except for formulations D2 (containing serine) and D7 (containing proline).

The formulations were assessed for presence of sub-particulates by Flowcam after five freeze-thaw cycles. The results are shown in Table 56. Sub-visible particles increased for most formulations.

TABLE 56

| | | | | |
|---|---|---|---|---|
| No. | 2-10 um | 10-25 um | 25-50 um | 50-100 um |

T0

| | | | | |
|---|---|---|---|---|
| D-CF | 612 | 124 | 0 | 0 |
| D1 | 428 | 184 | 152 | 0 |
| D2 | 460 | 152 | 92 | 31 |
| D3 | 400 | 216 | 60 | 0 |
| D4 | 244 | 60 | 0 | 31 |
| D5 | 520 | 184 | 60 | 0 |
| D6 | 980 | 552 | 276 | 31 |
| D7 | 948 | 244 | 60 | 0 |

FT

| | | | | |
|---|---|---|---|---|
| D-CF | 492 | 216 | 60 | 60 |
| D1 | 184 | 92 | 31 | 31 |
| D2 | 856 | 184 | 31 | 0 |
| D3 | 556 | 244 | 104 | 0 |
| D4 | 276 | 184 | 124 | 0 |
| D5 | 736 | 216 | 60 | 0 |
| D6 | 1408 | 276 | 152 | 0 |
| D7 | 1716 | 492 | 60 | 31 |

The formulations were subjected to five freeze-thaws and monitored by SEC-HPLC for total recovery, fusion protein recovery, and percent impurities consisting of HMW and LMW. The results are shown in Tables 57 and 58. Formulation D7 (containing proline) showed relatively higher HMW, demonstrating sensitivity to repeated freeze-thaw. The SEC data demonstrate that formulations containing arginine, lysine, serine, alanine, and glycine are relatively insensitive to repeated freeze-thaw, with nearly 100% recovery at T14.

TABLE 57

| | T0 | | | | | | |
|---|---|---|---|---|---|---|---|
| | % | % | % | HMW | Main | LMW | Total |
| No. | HMW | Main | LMW | AUC | AUC | AUC | AUC |
| D-CF | 1.2 | 71.9 | 26.9 | 134 | 7787 | 2910 | 10832 |
| D1 | 1.3 | 90.3 | 8.4 | 117 | 8203 | 761 | 9081 |
| D2 | 1.2 | 95.3 | 3.5 | 93 | 7543 | 275 | 7912 |
| D3 | 1.4 | 92.5 | 6.1 | 117 | 7569 | 498 | 8185 |
| D4 | 1.5 | 95.6 | 2.9 | 124 | 7837 | 237 | 8198 |
| D5 | 0.6 | 96.5 | 2.9 | 49 | 7758 | 236 | 8043 |
| D6 | 0.6 | 97.2 | 2.2 | 46 | 8050 | 182 | 8278 |
| D7 | 3.9 | 93.1 | 3.0 | 321 | 7625 | 241 | 8189 |

TABLE 58

| | Freeze-thaw | | | | | | | |
|---|---|---|---|---|---|---|---|---|
| No. | % HMW | % Main | % LMW | HMW AUC | Main AUC | LMW AUC | Total AUC | % total recovery |
| D-CF | 10.1 | 84.1 | 5.8 | 877 | 7338 | 507 | 8724 | 80.5 |
| D1 | 1.3 | 97.5 | 1.2 | 108 | 7943 | 96 | 8148 | 89.7 |
| D2 | 1.1 | 96.3 | 2.6 | 89 | 7759 | 208 | 8056 | 101.8 |
| D3 | 1.3 | 95.7 | 3.1 | 100 | 7620 | 244 | 7965 | 97.3 |
| D4 | 1.6 | 95.6 | 2.8 | 122 | 7504 | 222 | 7849 | 95.7 |
| D5 | 0.5 | 96.9 | 2.6 | 42 | 7900 | 210 | 8153 | 101.4 |
| D6 | 0.6 | 97.5 | 1.8 | 49 | 7963 | 150 | 8163 | 98.6 |
| D7 | 27.6 | 80.7 | 1.7 | 3128 | 8006 | 193 | 11329 | 138.3 |

The formulations that were subjected to five freeze-thaws were also analyzed by reducing and non-reducing SDS-PAGE. The results are shown in Table 59. Some LMW and HMW was observed after freeze-thaw, for example, in formulation D7 (containing proline).

TABLE 59

| FT Non-Reduced | | | | FT Reduced | | |
|---|---|---|---|---|---|---|
| No. | % Main | % LMW | % HMW | No | % Main | % LMW |
| D-CF | 98.09 | 1.91 | 0 | D-CF | 98.03 | 1.97 |
| D1 | 99.13 | 0.87 | 0 | D1 | 98.33 | 1.67 |
| D2 | 98.05 | 1.95 | 0 | D2 | 97.2 | 2.8 |
| D3 | 98.64 | 1.36 | 0 | D3 | 96.24 | 3.76 |
| D4 | 98.33 | 1.67 | 0 | D4 | 97.99 | 2.01 |
| D5 | 98.68 | 1.32 | 0 | D5 | 99 | 1 |
| D6 | 93.53 | 6.47 | 0 | D6 | 94.73 | 5.27 |
| D7 | 64.51 | 1.52 | 33.97 | D7 | 74.35 | 25.65 |

### 4.3. Simulated Shear Stress Study

The formulations at T0 were subjected to simulated shear stress for 24 hours at 900 RPM at room temperature and analysed for osmolality, pH, A280, RALS, SEC, and SDS-PAGE. The results for osmolality, pH, A280, and RALS are shown in Tables 60 and 61.

TABLE 60

| | Osmolality | | | | Concentration | | | |
|---|---|---|---|---|---|---|---|---|
| | (mOsm/kg) | | pH | | T0 | | Shear | |
| No. | T0 | Shear | T0 | Shear | A280 | μg/ml | A280 | μg/ml |
| D-CF | 30 | 3 | 7.52 | 7.54 | 0.033 | 21.54 | 0.048 | 31.33 |
| D1 | 44 | 43 | 7.53 | 7.51 | 0.034 | 22.19 | 0.042 | 27.42 |
| D2 | 83 | 84 | 7.48 | 7.51 | 0.029 | 18.93 | 0.135 | 88.12 |
| D3 | 78 | 77 | 7.55 | 7.58 | 0.033 | 21.54 | 0.068 | 44.39 |
| D4 | 78 | 79 | 7.51 | 7.51 | 0.029 | 18.93 | 0.079 | 51.57 |
| D5 | 121 | 121 | 7.51 | 7.45 | 0.035 | 22.85 | 0.068 | 44.39 |
| D6 | 122 | 122 | 7.53 | 7.53 | 0.032 | 20.89 | 0.074 | 48.30 |
| D7 | 80 | 82 | 7.52 | 7.54 | 0.03 | 19.58 | 0.04 | 26.11 |

As shown in Table 60, concentration increased after simulated shear stress, particularly for formulation D2 (containing serine).

TABLE 61
RALS

| | T0 | | | |
|---|---|---|---|---|
| | RALS | Shear RALS | | |
| | 565 | 565 | 515 | 465 |
| No. | Volts | Volts | Volts | Volts |
| D-CF | 1.08 | 7.732 | 2.737 | 0.771 |
| D1 | 1.12 | 6.501 | 2.202 | 0.636 |
| D2 | 1 | 9.999 | 6.822 | 1.94 |
| D3 | 1.13 | 4.174 | 1.451 | 0.412 |
| D4 | 1.26 | 9.999 | 5.525 | 1.544 |
| D5 | 1.08 | 9.999 | 6.6 | 1.925 |
| D6 | 1.07 | 9.999 | 6.524 | 1.893 |
| D7 | 1.25 | 4.632 | 1.585 | 0.453 |

As shown in Table 61, RALS values increased after simulated shear stress across all formulations. Formulations containing alanine and proline (D3 and D7) had the lowest RALS values, indicating improved stability during shear.

The formulations were subjected to simulated shear stress for 24 hours at 900 RPM and monitored by SEC-HPLC for total recovery, fusion protein recovery, and percent impurities consisting of HMW and LMW. The results are shown in Tables 62 and 63. The results demonstrated that the formulations containing serine and alanine (D2 and D3) exhibited higher total recovery after simulated shear stress, indicating relatively less sensitivity to shear stress.

TABLE 62

| | T0 | | | | | | |
|---|---|---|---|---|---|---|---|
| | % | % | % | HMW | Main | LMW | Total |
| No. | HMW | Main | LMW | AUC | AUC | AUC | AUC |
| D-CF | 1.2 | 71.9 | 26.9 | 134 | 7787 | 2910 | 10832 |
| D1 | 1.3 | 90.3 | 8.4 | 117 | 8203 | 761 | 9081 |
| D2 | 1.2 | 95.3 | 3.5 | 93 | 7543 | 275 | 7912 |
| D3 | 1.4 | 92.5 | 6.1 | 117 | 7569 | 498 | 8185 |
| D4 | 1.5 | 95.6 | 2.9 | 124 | 7837 | 237 | 8198 |
| D5 | 0.6 | 96.5 | 2.9 | 49 | 7758 | 236 | 8043 |
| D6 | 0.6 | 97.2 | 2.2 | 46 | 8050 | 182 | 8278 |
| D7 | 3.9 | 93.1 | 3.0 | 321 | 7625 | 241 | 8189 |

TABLE 63

| | | | | | | | | % |
|---|---|---|---|---|---|---|---|---|
| | | | | | | | | total |
| | Simulated Shear Stress | | | | | | | re- |
| | % | % | % | HMW | Main | LMW | Total | cov- |
| No. | HMW | Main | LMW | AUC | AUC | AUC | AUC | ery |
| D-CF | 67.2 | 29.2 | 3.6 | 3637 | 1579 | 197 | 5415 | 50.0 |
| D1 | 73.9 | 25.6 | 0.4 | 3506 | 1216 | 21 | 4744 | 52.2 |
| D2 | 74.2 | 21.7 | 4.2 | 6882 | 2011 | 388 | 9284 | 117.3 |
| D3 | 65.5 | 33.8 | 0.7 | 6177 | 3194 | 63 | 9439 | 115.3 |
| D4 | 65.3 | 30.1 | 4.5 | 4665 | 2151 | 324 | 7142 | 87.1 |
| D5 | 48.3 | 32.2 | 19.5 | 1233 | 823 | 497 | 2555 | 31.8 |
| D6 | 61.8 | 37.7 | 0.5 | 2715 | 1654 | 21 | 4391 | 53.0 |
| D7 | 52.9 | 45.3 | 1.7 | 3564 | 3052 | 115 | 6735 | 82.2 |

The formulations that were subjected to simulated shear stress were also analysed by reducing and non-reducing SDS-PAGE. The results are shown in Table 64. HMW and LMW were observed in all formulations during shear stress on gels. Formulations containing arginine and proline (D5 and D7) demonstrated relatively lower percentages of HMW and LMW during shear stress, indicating improved stability.

TABLE 64

| Shear Non-Reduced | | | | Shear Reduced | | | |
|---|---|---|---|---|---|---|---|
| | % | % | % | | % | % | % |
| No. | Main | HMW | LMW | No. | Main | HMW | LMW |
| D-CF | 85.72 | 12.57 | 1.71 | D-CF | 97.73 | 0 | 2.27 |
| D1 | 81.84 | 16.63 | 1.53 | D1 | 96.54 | 0.59 | 2.88 |
| D2 | 61.04 | 34.97 | 3.99 | D2 | 67.58 | 4.94 | 27.48 |
| D3 | 58.6 | 40.04 | 1.35 | D3 | 77.12 | 3.95 | 18.93 |
| D4 | 76.51 | 16.01 | 7.48 | D4 | 82.88 | 0 | 17.12 |
| D5 | 85.09 | 11.05 | 3.85 | D5 | 92.14 | 5.6 | 5.6 |
| D6 | 85.4 | 13.1 | 1.5 | D6 | 94.53 | 4.24 | 4.24 |
| D7 | 94.59 | 4.22 | 1.19 | D7 | 95.01 | 4.99 | 4.99 |

Based on SEC data with thermal stress, the formulations containing arginine and lysine (D5 and D6 containing basic amino acids) demonstrated relatively more stability compared to other formulations. The formulation containing proline (D7), however, demonstrated a large amount of HMW after freeze-thaw. The formulation containing arginine (D5) demonstrated the most amount of LMW during simulated shear stress, indicating relatively more sensitivity for shear stress. Further, the results suggest that the formulations containing alanine and proline (D3 and D7) protect against agitation/simulated shear stress, whereas formulations containing arginine and lysine (D5 and D6) protect against thermal stress.

### Example 5: Stability Studies for Formulations with Varying Carbohydrate Stabilizers, Cryoprotectants and/or Lyoprotectants

Formulations of SONE-IL12-ABD were prepared as shown in Table 65 using a SONE-IL12-ABD concentration of approximately 20 μg/ml.

TABLE 65

| No. | Formulation |
|---|---|
| E-CF | 25 mM GlyGly, 10 μM DTPA, 0.02% PS20, pH 7.5 |
| E1 | 280 mM trehalose, 25 mM GlyGly, 10 μM DTPA, 0.02% PS20 |
| E2 | 50 mM alanine, 240 mM trehalose, 25 mM GlyGly, 10 μM |
| | DTPA, 0.02% PS20 |
| E3 | 50 mM alanine, 240 mM sucrose, 25 mM GlyGly, 10 μM |
| | DTPA, 0.02% PS20 |
| E4 | 50 mM alanine, 240 mM mannitol, 25 mM GlyGly, 10 μM |
| | DTPA, 0.02% PS20 |
| E5 | 50 mM alanine, 120 mM sucrose, 120 mM mannitol, 25 mM |
| | GlyGly, 10 μM DTPA, 0.02% PS20 |
| E6 | 50 mM arginine, 240 mM trehalose, 25 mM GlyGly, 10 μM |
| | DTPA, 0.02% PS20 |
| E7 | 50 mM proline, 240 mM trehalose, 25 mM GlyGly, 10 μM |
| | DTPA, 0.02% PS20 |
| E8 | 50 mM alanine, 50 mM arginine, 190 mM trehalose, 25 mM |
| | GlyGly, 10 μM DTPA, 0.02% PS20 |

The formulations shown in Table 65 were also lyophilized according to the lyophilization cycle shown in Table 66.

TABLE 66

| | | Temperature | |
|---|---|---|---|
| Step | Temperature | Ramp (° C./min) | Pressure |
| Loading | ambient | N/A | NA |
| Freeze | −50° C. | 1° C./min. | NA |
| Primary Drying | −20° C. | 1° C./min | 70 mTorr |
| Secondary Drying | +20° C. | 1° C./min | 70 mTorr |

The liquid formulations were subjected to temperature ramping RALS and IF to find Tagg and Tm of each formulation. Tm and Tagg values are shown in Table 67. Tm and Tagg are similar across all formulations. Formulations containing arginine and trehalose (E6), and alanine, arginine, and trehalose (E8), have the highest Tm.

TABLE 67

| No | Tm (° C.) | Tagg (° C.) |
|---|---|---|
| E-CF | 46 | 69 |
| E1 | 49 | 68 |
| E2 | 46 | 66 |
| E3 | 47 | 69 |
| E4 | 48 | 68 |
| E5 | 48 | 68 |
| E6 | 53 | 67 |
| E7 | 49 | 67 |
| E8 | 54 | 66 |

### 5.1. Thermal Stress Study

The liquid and lyophilized formulations were thermally stressed for 14 days at 40° C. and monitored for visual appearance, osmolality, pH, A280, RALS, Flowcam, SEC-HPLC, reducing and non-reducing SDS-page, and ELISA. The results for visual appearance are shown in Table 68, for osmolality, pH, and RALS in Tables 69 and 70, and for A280 in Tables 71 and 72.

As shown in Table 68, all liquid and reconstituted samples were clear, colorless, and had no visible particles. The lyophilized cake for the formulation containing mannitol (E4) had the no cracks or pulling away from the glass. The lyophilized cakes for E-CF and for the formulation containing alanine, sucrose, and mannitol (E5) were completely collapsed at T14

TABLE 68

| | T0, T7, T14 | T0, T7, T14 | T0, T7, T14 lyo |
|---|---|---|---|
| No. | liquid | lyophilized cake | reconstituted |
| E-CF | Clear, colorless, no | White, completely | Clear, colorless, no |
| | visible particles | collapsed cake (No | visible particles |
| | | change from T0) | |
| E1 | Clear, colorless, no | White, 10% pulling | Clear, colorless, no |
| | visible particles | from glass (No | visible particles |
| | | change from T0) | |
| E2 | Clear, colorless, no | White, 10% pulling | Clear, colorless, no |
| | visible particles | from glass (No | visible particles |
| | | change from T0) | |
| E3 | Clear, colorless, no | White, 50% pulling | Clear, colorless, no |
| | visible particles | away from glass, | visible particles |
| | | very shrunken at | |
| | | T14 | |
| E4 | Clear, colorless, no | White, not | Clear, colorless, no |
| | visible particles | collapsed, | visible particles |
| | | no cracks, | |
| | | no pulling away | |
| | | from glass (No | |
| | | change from T0) | |
| E5 | Clear, colorless, no | White, completely | Clear, colorless, no |
| | visible particles | collapsed at | visible particles |
| | | T14 | |
| E6 | Clear, colorless, no | White, cracked, | Clear, colorless, no |
| | visible particles | 10% shrinkage, (No | visible particles |
| | | change from T0) | |
| E7 | Clear, colorless, no | White, 20% pulling | Clear, colorless, no |
| | visible particles | away from glass (No | visible particles |
| | | change from T0) | |
| E8 | Clear, colorless, no | White, 10% pulling | Clear, colorless, no |
| | visible particles | from glass (No | visible particles |
| | | change form T0) | |

TABLE 69

| | Osmolality | | | | | | RALS | | |
|---|---|---|---|---|---|---|---|---|---|
| No. | (mOsm/kg) | | | pH | | | (565 Volts) | | |
| (liquid) | T0 | T7 | T14 | T0 | T7 | T14 | T0 | T7 | T14 |
| E-CF | 30 | 30 | 32 | 7.54 | 7.58 | 7.56 | 0.764 | 0.718 | 0.675 |
| E1 | 348 | 349 | 349 | 7.52 | 7.45 | 7.44 | 1.416 | 1.297 | 1.412 |
| E2 | 347 | 349 | 346 | 7.53 | 7.5 | 7.5 | 1.337 | 1.275 | 1.250 |
| E3 | 348 | 349 | 347 | 7.54 | 7.5 | 7.48 | 1.656 | 1.289 | 1.355 |
| E4 | 332 | 335 | 334 | 7.53 | 7.58 | 7.62 | 0.942 | 0.889 | 0.800 |
| E5 | 340 | 343 | 342 | 7.52 | 7.47 | 7.45 | 1.401 | 1.219 | 1.101 |
| E6 | 384 | 386 | 380 | 7.57 | 7.65 | 7.65 | 1.339 | 1.304 | 1.222 |
| E7 | 353 | 353 | 353 | 7.58 | 7.56 | 7.55 | 1.404 | 1.230 | 1.252 |
| E8 | 381 | 385 | 384 | 7.57 | 7.63 | 7.61 | 1.255 | 1.133 | 1.152 |

TABLE 70

| No. | Osmolality | | | | | | RALS | | |
|---|---|---|---|---|---|---|---|---|---|
| (lyoph- | (mOsm/kg) | | | pH | | | (565 Volts) | | |
| ilized) | T0 | T7 | T14 | T0 | T7 | T14 | T0 | T7 | T14 |
| E-CF | 31 | 31 | 33 | 7.58 | 7.57 | 7.60 | 0.844 | 0.792 | 0.909 |
| E1 | 342 | 345 | 343 | 7.53 | 7.46 | 7.50 | 1.484 | 1.384 | 1.441 |
| E2 | 342 | 346 | 342 | 7.48 | 7.53 | 7.52 | 1.475 | 1.485 | 1.665 |
| E3 | 342 | 348 | 346 | 7.50 | 7.50 | 7.45 | 1.745 | 1.522 | 2.661 |
| E4 | 341 | 345 | 337 | 7.57 | 7.60 | 7.60 | 0.888 | 1.275 | 2.734 |
| E5 | 342 | 341 | 344 | 7.55 | 7.48 | 7.47 | 1.230 | 1.105 | 2.009 |
| E6 | 381 | 385 | 380 | 7.63 | 7.66 | 7.63 | 1.472 | 1.332 | 2.154 |
| E7 | 353 | 353 | 349 | 7.58 | 7.53 | 7.56 | 1.378 | 1.592 | 1.390 |
| E8 | 376 | 380 | 379 | 7.61 | 7.64 | 7.60 | 1.459 | 1.644 | 1.414 |

TABLE 71

| | | | | | % Re- | | | % Re- |
|---|---|---|---|---|---|---|---|---|
| No. | T0 | | T7 | | covery | T14 | | covery |
| (liquid) | A280 | μg/ml | A280 | μg/ml | at T7 | A280 | μg/ml | at T14 |
| E-CF | 0.034 | 22.19 | 0.032 | 20.89 | 94.14 | 0.034 | 22.19 | 100 |
| E1 | 0.036 | 23.5 | 0.03 | 19.58 | 83.32 | 0.029 | 18.93 | 80.55 |
| E2 | 0.037 | 24.15 | 0.032 | 20.89 | 86.5 | 0.032 | 20.89 | 86.5 |
| E3 | 0.037 | 24.15 | 0.03 | 19.58 | 81.08 | 0.031 | 20.23 | 83.77 |
| E4 | 0.034 | 22.19 | 0.032 | 20.89 | 94.14 | 0.03 | 19.58 | 88.24 |
| E5 | 0.037 | 24.15 | 0.03 | 19.58 | 81.08 | 0.038 | 24.8 | 102.69 |
| E6 | 0.034 | 22.19 | 0.037 | 24.15 | 108.83 | 0.033 | 21.54 | 97.07 |
| E7 | 0.04 | 26.11 | 0.035 | 22.85 | 87.51 | 0.035 | 22.85 | 87.51 |
| E8 | 0.033 | 21.54 | 0.034 | 22.19 | 103.02 | 0.031 | 20.23 | 93.92 |

TABLE 72

| No. | | | | | % Re- | | | % Re- |
|---|---|---|---|---|---|---|---|---|
| (lyoph- | T0 | | T7 | | covery | T14 | | covery |
| ilized) | A280 | μg/ml | A280 | μg/ml | at T7 | A280 | μg/ml | at T14 |
| E-CF | 0.036 | 23.5 | 0.04 | 26.11 | 111.11 | 0.038 | 24.8 | 105.53 |
| E1 | 0.029 | 18.93 | 0.032 | 20.89 | 110.35 | 0.034 | 22.19 | 117.22 |
| E2 | 0.029 | 18.93 | 0.035 | 22.85 | 120.71 | 0.031 | 20.23 | 106.87 |
| E3 | 0.036 | 23.5 | 0.032 | 20.89 | 88.89 | 0.032 | 20.89 | 88.89 |
| E4 | 0.03 | 19.58 | 0.038 | 24.8 | 126.66 | 0.036 | 23.50 | 120.02 |
| E5 | 0.031 | 20.23 | 0.039 | 25.46 | 125.85 | 0.043 | 28.07 | 138.75 |
| E6 | 0.033 | 21.54 | 0.035 | 22.85 | 106.08 | 0.035 | 22.85 | 106.08 |
| E7 | 0.033 | 21.54 | 0.039 | 25.46 | 118.2 | 0.034 | 22.19 | 103.02 |
| E8 | 0.034 | 22.19 | 0.04 | 26.11 | 117.67 | 0.034 | 22.19 | 100 |

As shown in Table 70, there was a slight increase in RALS for the lyophilized formulation containing alanine and sucrose (E3), the lyophilized formulation containing alanine and mannitol (E4), the lyophilized formulation containing alanine, sucrose, and mannitol (E5), and the lyophilized formulation containing arginine and trehalose (E6).

The formulations were assessed for presence of sub-particulates by Flowcam after incubation for 7 and 14 days at 40° C. As shown in Table 73, sub-visible particle counts increased for all liquid formulations after incubation for 14 days, with the formulation containing proline (E7) having relatively higher particulate counts. As shown in Table 74, sub-visible particle counts increased for all lyophilized formulations after incubation for 14 days, with the formulation containing proline and trehalose (E7) having relatively higher particulate counts.

TABLE 73

| | T0 (liquid) | | | | | T7 (liquid) | | | | T14 (liquid) | | | |
|---|---|---|---|---|---|---|---|---|---|---|---|---|---|
| | 2-10 | 10-25 | 25-50 | 50-100 | 100 | 2-10 | 10-25 | 25-50 | 50-100 | 2-10 | 10-25 | 25-50 | 50-100 |
| No. | um | um | um | um | um+ | um | um | um | um | um | um | um | um |
| E-CF | 580 | 152 | 92 | 0 | 0 | 620 | 256 | 72 | 0 | 3844 | 888 | 0 | 0 |
| E1 | 1684 | 152 | 92 | 0 | 0 | 1196 | 184 | 0 | 0 | 3924 | 252 | 0 | 0 |
| E2 | 888 | 276 | 92 | 31 | 31 | 1900 | 764 | 336 | 31 | 6980 | 996 | 0 | 0 |
| E3 | 152 | 184 | 0 | 31 | 0 | 584 | 276 | 60 | 0 | 2144 | 632 | 128 | 0 |
| E4 | 460 | 152 | 60 | 0 | 0 | 772 | 284 | 40 | 0 | 4044 | 860 | 244 | 0 |
| E5 | 588 | 124 | 216 | 0 | 0 | 976 | 116 | 116 | 0 | 5316 | 1580 | 0 | 0 |
| E6 | 1992 | 276 | 124 | 0 | 0 | 1916 | 372 | 0 | 0 | 7216 | 648 | 128 | 0 |
| E7 | 1472 | 124 | 31 | 0 | 0 | 2296 | 124 | 31 | 0 | 14332 | 636 | 0 | 0 |
| E8 | 764 | 184 | 92 | 0 | 0 | 888 | 152 | 31 | 0 | 2708 | 644 | 128 | 0 |

TABLE 74

| | T0 (lyophilized) | | | | | T7 (lyophilized) | | | | T14 (lyophilized) | | | |
|---|---|---|---|---|---|---|---|---|---|---|---|---|---|
| | 2-10 | 10-25 | 25-50 | 50-100 | 100 | 2-10 | 10-25 | 25-50 | 50-100 | 2-10 | 10-25 | 25-50 | 50-100 |
| No. | um | um | um | um | um+ | um | um | um | um | um | um | um | um |
| E-CF | 796 | 152 | 152 | 0 | 0 | 1532 | 400 | 60 | 0 | 4256 | 548 | 0 | 0 |
| E1 | 736 | 208 | 156 | 0 | 0 | 644 | 216 | 152 | 0 | 5024 | 368 | 0 | 0 |
| E2 | 1924 | 532 | 244 | 0 | 40 | 1520 | 440 | 168 | 0 | 7784 | 1624 | 0 | 0 |
| E3 | 1288 | 428 | 152 | 0 | 0 | 400 | 124 | 60 | 0 | 9944 | 1008 | 0 | 0 |
| E4 | 660 | 220 | 0 | 0 | 0 | 1560 | 232 | 132 | 0 | 9436 | 368 | 0 | 0 |
| E5 | 1716 | 520 | 124 | 0 | 0 | 1156 | 384 | 39 | 0 | 8244 | 1288 | 428 | 124 |
| E6 | 3944 | 480 | 404 | 0 | 0 | 1928 | 168 | 100 | 33 | 7880 | 256 | 0 | 0 |
| E7 | 1412 | 152 | 92 | 92 | 0 | 3600 | 668 | 332 | 0 | 18964 | 336 | 216 | 32 |
| E8 | 3424 | 928 | 352 | 32 | 0 | 2172 | 424 | 0 | 0 | 4044 | 368 | 0 | 0 |

The formulations were thermally stressed for 14 days at 40° C. and monitored by SEC-HPLC for total recovery, fusion protein recovery, and percent impurities consisting of HMW and LMW. The results are shown in Tables 75 to 80.

The liquid formulation containing arginine and trehalose (E6) and the liquid formulation containing alanine, arginine, and trehalose (E8) had no change in % HMW on SEC after incubation for 14 days. The results demonstrated that the presence of arginine protected the liquid formulations against thermal stress. The % LMW was lowest for the liquid formulation containing alanine, sucurose, and mannitol (E5). The % Main peak at T14 was highest for the liquid formulation containing arginine and trehalose (E6) and the liquid formulation containing alanine, arginine, and trehalose (E8). The results also demonstrated that the total recovery was relatively higher for the liquid formulation containing alanine and trehalose (E2) and the liquid formulation containing alanine, arginine, and trehalose (E8).

The lyophilized formulations were relatively more stable for 14 days at 40° C. with respect to total recovery and main peak recovery. The % HMW was slightly higher for E-CF and the main peak recovery and % LMW at T14 was relatively lower for the formulation containing arginine and trehalose (E6).

TABLE 75

| | T0 (liquid) | | | | | | |
|---|---|---|---|---|---|---|---|
| | % | % | % | HMW | Main | LMW | Total |
| No. | HMW | Main | LMW | AUC | AUC | AUC | AUC |
| E-CF | 2.0 | 89.6 | 8.3 | 219 | 9572 | 890 | 10681 |
| E1 | 3.6 | 95.7 | 0.7 | 336 | 8941 | 69 | 9346 |
| E2 | 1.8 | 97.2 | 1.0 | 164 | 8858 | 89 | 9111 |
| E3 | 1.9 | 97.3 | 0.8 | 173 | 8913 | 77 | 9163 |
| E4 | 1.7 | 97.7 | 0.6 | 152 | 8903 | 56 | 9111 |
| E5 | 1.8 | 97.3 | 0.9 | 163 | 8868 | 80 | 9111 |
| E6 | 1.6 | 96.6 | 1.8 | 148 | 8871 | 164 | 9183 |
| E7 | 1.8 | 96.8 | 1.4 | 182 | 9662 | 138 | 9982 |
| E8 | 1.2 | 98.3 | 0.5 | 111 | 9286 | 48 | 9445 |

TABLE 76

| | T7 (liquid) | | | | | | | |
|---|---|---|---|---|---|---|---|---|
| | | | | | | | | % |
| | | | | | | | | total |
| | | | | | | | | re- |
| | % | % | % | HMW | Main | LMW | Total | cov- |
| No. | HMW | Main | LMW | AUC | AUC | AUC | AUC | ery |
| E-CF | 14.5 | 82.5 | 3.0 | 1384 | 7868 | 289 | 9541 | 89.3 |
| E1 | 9.4 | 84.7 | 6.0 | 863 | 7790 | 550 | 9203 | 98.5 |
| E2 | 9.7 | 84.9 | 5.4 | 874 | 7677 | 492 | 9043 | 99.3 |
| E3 | 10.9 | 84.2 | 4.8 | 976 | 7513 | 429 | 8918 | 97.3 |
| E4 | 10.1 | 87.2 | 2.7 | 863 | 7480 | 236 | 8579 | 94.2 |
| E5 | 11.3 | 86.1 | 2.6 | 928 | 7037 | 210 | 8175 | 89.7 |
| E6 | 1.1 | 96.2 | 2.7 | 100 | 8787 | 244 | 9131 | 99.4 |
| E7 | 9.0 | 88.6 | 2.4 | 851 | 8415 | 231 | 9497 | 95.1 |
| E8 | 1.1 | 96.3 | 2.6 | 99 | 8825 | 242 | 9166 | 97.0 |

TABLE 77

| | T14 (liquid) | | | | | | | |
|---|---|---|---|---|---|---|---|---|
| | | | | | | | | % |
| | | | | | | | | Total |
| | | | | | | | | re- |
| | % | % | % | HMW | Main | LMW | Total | cov- |
| No. | HMW | Main | LMW | AUC | AUC | AUC | AUC | ery |
| E-CF | 18.7 | 75.4 | 5.8 | 1780 | 7163 | 552 | 9495 | 88.9 |
| E1 | 13.9 | 79.8 | 6.2 | 1247 | 7135 | 559 | 8941 | 95.7 |
| E2 | 14.0 | 79.9 | 6.1 | 1244 | 7076 | 538 | 8858 | 97.2 |
| E3 | 16.7 | 78.9 | 4.4 | 1385 | 6554 | 369 | 8308 | 90.7 |
| E4 | 14.2 | 82.1 | 3.6 | 1218 | 7020 | 309 | 8547 | 93.8 |
| E5 | 17.0 | 79.7 | 3.3 | 1363 | 6392 | 269 | 8024 | 88.1 |
| E6 | 1.5 | 95.0 | 3.5 | 126 | 7883 | 289 | 8298 | 90.4 |
| E7 | 15.4 | 81.2 | 3.4 | 1228 | 6498 | 272 | 7998 | 80.1 |
| E8 | 1.2 | 94.6 | 4.2 | 110 | 8843 | 397 | 9350 | 99.0 |

TABLE 78

| | T0 (lyophilized) | | | | | | | |
|---|---|---|---|---|---|---|---|---|
| | | | | | | | | % total |
| | % | % | % | HMW | Main | LMW | Total | recovery |
| No. | HMW | Main | LMW | AUC | AUC | AUC | AUC | from T0 liquid |
| E-CF | 1.4 | 91.7 | 7.0 | 147 | 9714 | 737 | 10598 | 99.2 |
| E1 | 1.3 | 93.5 | 5.2 | 115 | 8358 | 463 | 8936 | 95.6 |
| E2 | 1.5 | 97.0 | 1.4 | 134 | 8422 | 124 | 8680 | 95.3 |
| E3 | 1.6 | 96.5 | 2.0 | 132 | 8229 | 167 | 8528 | 93.1 |
| E4 | 1.3 | 96.5 | 2.2 | 116 | 8622 | 193 | 8931 | 98.0 |
| E5 | 1.3 | 96.8 | 2.0 | 110 | 8472 | 172 | 8754 | 96.1 |
| E6 | 1.2 | 95.9 | 2.9 | 111 | 8895 | 272 | 9278 | 101.0 |
| E7 | 1.4 | 95.6 | 3.0 | 137 | 9514 | 301 | 9952 | 99.7 |
| E8 | 1.4 | 95.9 | 2.7 | 125 | 8586 | 246 | 8957 | 94.8 |

TABLE 79

| | T7 (lyophilized) | | | | | | | |
|---|---|---|---|---|---|---|---|---|
| | % | % | % | HMW | Main | LMW | Total | % total |
| No. | HMW | Main | LMW | AUC | AUC | AUC | AUC | recovery |
| E-CF | 3.4 | 90.9 | 5.7 | 342 | 9298 | 583 | 10223 | 96.5 |
| E1 | 0.8 | 93.7 | 5.4 | 81 | 9064 | 527 | 9672 | 108.2 |
| E2 | 0.8 | 94.8 | 4.3 | 79 | 8961 | 409 | 9449 | 108.9 |
| E3 | 1.0 | 96.8 | 2.2 | 88 | 8978 | 207 | 9273 | 108.7 |
| E4 | 1.5 | 96.7 | 1.8 | 132 | 8256 | 152 | 8540 | 95.6 |
| E5 | 0.9 | 96.2 | 3.0 | 77 | 8460 | 260 | 8797 | 100.5 |
| E6 | 0.7 | 96.6 | 2.7 | 69 | 9181 | 258 | 9508 | 102.5 |
| E7 | 0.8 | 96.3 | 2.9 | 74 | 8953 | 271 | 9298 | 93.4 |
| E8 | 0.7 | 96.2 | 3.1 | 69 | 9328 | 296 | 9693 | 108.2 |

TABLE 80

| | T14 (lyophilized) | | | | | | | |
|---|---|---|---|---|---|---|---|---|
| | % | % | % | HMW | Main | LMW | Total | % Total |
| No. | HMW | Main | LMW | AUC | AUC | AUC | AUC | recovery |
| E-CF | 3.2 | 88.6 | 8.2 | 331 | 9240 | 855 | 10426 | 98.4 |
| E1 | 0.9 | 90.2 | 8.9 | 89 | 9201 | 907 | 10197 | 114.1 |
| E2 | 0.9 | 91.2 | 7.9 | 88 | 8772 | 762 | 9622 | 110.9 |
| E3 | 1.1 | 92.5 | 6.4 | 108 | 8854 | 609 | 9571 | 112.2 |
| E4 | 1.7 | 93.2 | 5.1 | 161 | 9044 | 500 | 9705 | 108.7 |
| E5 | 1.3 | 92.4 | 6.3 | 125 | 8678 | 593 | 9396 | 107.3 |
| E6 | 0.9 | 94.1 | 5.0 | 90 | 9097 | 485 | 9672 | 104.2 |
| E7 | 1.0 | 93.5 | 5.5 | 105 | 9903 | 580 | 10588 | 106.4 |
| E8 | 0.8 | 93.6 | 5.6 | 80 | 9520 | 569 | 10169 | 113.5 |

The liquid and lyophilized formulations E1 (trehalose), E2 (alanine and trehalose), E4 (alanine and mannitol), and E8 (alanine, arginine, and trehalose) that were thermally stressed for 14 days at 40° C. were also analyzed by reducing and non-reducing SDS-PAGE. Qualitative analysis by silver staining indicated that formulation E2 (alanine and trehalose) demonstrated the lowest impurities.

Liquid and lyophilized formulations E1 (trehalose), E2 (alanine and trehalose), E4 (alanine and mannitol), and E8 (alanine, arginine, and trehalose) were also analysed by ELISA. The results are shown in Table 81.

TABLE 81

| No. | Timepoint | Avg [C] μg/mL |
|---|---|---|
| E1 (liquid) | T0 | 21.81 |
| | T14 | 8.01 |
| E1 (lyophilized) | T0 | 20.97 |
| | T14 | 23.55 |
| E2 (liquid) | T0 | 19.68 |
| | T7 | 9.93 |
| | T14 | 8.29 |
| E2 (lyophilized) | T0 | 20.20 |
| | T7 | 18.52 |
| | T14 | 21.78 |
| E4 (liquid) | T0 | 22.96 |
| | T14 | 10.28 |
| E4 (lyophilized) | T0 | 22.60 |
| | T14 | 22.64 |
| E8 (liquid) | T0 | 31.16 |
| | T14 | 17.84 |
| E8 (lyophilized) | T0 | 21.77 |
| | T14 | 19.42 |

### 5.2. Freeze-Thaw Study

The formulations at T0 were subjected to five freeze-thaws and then analyzed for osmolality, pH, RALS, A280, Flowcam, and SEC-HPLC. The results for osmolality, pH, A280, and RALS are shown in Table 81A.

TABLE 81A

| | Osmolality | | | | | | | | | RALS | |
|---|---|---|---|---|---|---|---|---|---|---|---|
| | (mOsm/kg) | | pH | | Concentration | | | | | (565 Volts) | |
| | T0 | Freeze- | T0 | Freeze- | T0 liquid | | Freeze-thaw | | % | | Freeze- |
| No. | liquid | thaw | liquid | thaw | A280 | μg/ml | A280 | μg/ml | recovery | T0 | thaw |
| E-CF | 30 | 31 | 7.54 | 7.53 | 0.034 | 22.19 | 0.034 | 22.19 | 100.0 | 0.764 | 0.818 |
| E1 | 348 | 350 | 7.52 | 7.49 | 0.036 | 23.5 | 0.031 | 20.23 | 86.1 | 1.416 | 1.396 |
| E2 | 347 | 348 | 7.53 | 7.49 | 0.037 | 24.15 | 0.031 | 20.23 | 83.8 | 1.337 | 1.33 |
| E3 | 348 | 348 | 7.54 | 7.5 | 0.037 | 24.15 | 0.032 | 20.89 | 86.5 | 1.656 | 1.437 |
| E4 | 332 | 332 | 7.53 | 7.56 | 0.034 | 22.19 | 0.03 | 19.58 | 88.2 | 0.942 | 1.016 |
| E5 | 340 | 344 | 7.52 | 7.53 | 0.037 | 24.15 | 0.031 | 20.23 | 83.8 | 1.401 | 1.154 |
| E6 | 384 | 388 | 7.57 | 7.62 | 0.034 | 22.19 | 0.029 | 18.93 | 85.3 | 1.339 | 1.937 |
| E7 | 353 | 355 | 7.58 | 7.55 | 0.04 | 26.11 | 0.037 | 24.15 | 92.5 | 1.404 | 1.877 |
| E8 | 381 | 386 | 7.57 | 7.6 | 0.033 | 21.54 | 0.03 | 19.58 | 90.9 | 1.255 | 1.159 |

As shown in Table 81A, concentration decreased slightly after freeze-thaw, except for formulations E-CF.

The formulations were assessed for presence of sub-particulates by Flowcam after five freeze-thaw cycles. The results are shown in Table 82. Sub-visible particles in the 2-10 μm range increased for all formulations.

TABLE 82

| | T0 liquid | | | | | Freeze-thaw | | | | |
|---|---|---|---|---|---|---|---|---|---|---|
| | 2-10 | 10-25 | 25-50 | 50-100 | 100 | 2-10 | 10-25 | 25-50 | 50-100 | 100 |
| No. | um | um | um | um | um+ | um | um | um | um | um+ |
| E-CF | 580 | 152 | 92 | 0 | 0 | 828 | 276 | 60 | 0 | 0 |
| E1 | 1684 | 152 | 92 | 0 | 0 | 1132 | 124 | 31 | 31 | 0 |
| E2 | 888 | 276 | 92 | 31 | 31 | 1316 | 276 | 124 | 0 | 0 |
| E3 | 152 | 184 | 0 | 31 | 0 | 520 | 60 | 0 | 0 | 0 |
| E4 | 460 | 152 | 60 | 0 | 0 | 1072 | 216 | 0 | 0 | 60 |
| E5 | 588 | 124 | 216 | 0 | 0 | 5220 | 716 | 132 | 33 | 0 |
| E6 | 1992 | 276 | 124 | 0 | 0 | 1704 | 240 | 34 | 0 | 0 |
| E7 | 1472 | 124 | 31 | 0 | 0 | 3308 | 292 | 72 | 0 | 0 |
| E8 | 764 | 184 | 92 | 0 | 0 | 676 | 124 | 0 | 0 | 0 |

The formulations were subjected to five freeze-thaws and monitored by SEC-HPLC for total recovery, fusion protein recovery, and percent impurities consisting of HMW and LMW. The results are shown in Tables 83 and 84. The % HMW was similar across the formulations, except for E-CF and the formulation containing trehalose (E1), which had relatively lower HMW species after freeze-thaw. The formulation containing alanine and sucrose (E3) and the formulation containing alanine, sucrose, and mannitol (E5) presented the lowest amount of % LMW. Formulation E6 containing alanine, arginine, and trehalose presented the lowest % recovery after freeze-thaw.

TABLE 83

| | T0 liquid | | | | | | |
|---|---|---|---|---|---|---|---|
| | % | % | % | HMW | Main | LMW | Total |
| No. | HMW | Main | LMW | AUC | AUC | AUC | AUC |
| E-CF | 2.0 | 89.6 | 8.3 | 219 | 9572 | 890 | 10681 |
| E1 | 3.6 | 95.7 | 0.7 | 336 | 8941 | 69 | 9346 |
| E2 | 1.8 | 97.2 | 1.0 | 164 | 8858 | 89 | 9111 |
| E3 | 1.9 | 97.3 | 0.8 | 173 | 8913 | 77 | 9163 |
| E4 | 1.7 | 97.7 | 0.6 | 152 | 8903 | 56 | 9111 |
| E5 | 1.8 | 97.3 | 0.9 | 163 | 8868 | 80 | 9111 |
| E6 | 1.6 | 96.6 | 1.8 | 148 | 8871 | 164 | 9183 |
| E7 | 1.8 | 96.8 | 1.4 | 182 | 9662 | 138 | 9982 |
| E8 | 1.2 | 98.3 | 0.5 | 111 | 9286 | 48 | 9445 |

TABLE 84

| | Freeze-thaw | | | | | | | |
|---|---|---|---|---|---|---|---|---|
| | % | % | % | HMW | Main | LMW | Total | % total |
| No. | HMW | Main | LMW | AUC | AUC | AUC | AUC | recovery |
| E-CF | 0.6 | 87.1 | 12.3 | 72 | 10039 | 1412 | 11523 | 107.9 |
| E1 | 1.3 | 93.1 | 5.7 | 122 | 9028 | 553 | 9703 | 103.8 |
| E2 | 1.4 | 95.5 | 3.1 | 127 | 8808 | 288 | 9223 | 101.2 |
| E3 | 1.5 | 95.8 | 2.8 | 134 | 8767 | 252 | 9153 | 99.9 |
| E4 | 1.5 | 95.7 | 2.8 | 139 | 8825 | 262 | 9226 | 101.3 |
| E5 | 1.3 | 95.2 | 3.5 | 121 | 8819 | 325 | 9265 | 101.7 |
| E6 | 1.1 | 92.2 | 6.7 | 81 | 6782 | 495 | 7358 | 80.1 |
| E7 | 1.1 | 92.4 | 6.5 | 115 | 9456 | 665 | 10236 | 102.5 |
| E8 | 1.2 | 95.3 | 3.5 | 110 | 8980 | 334 | 9424 | 99.8 |

### 5.3. Simulated Shear Stress Study

The formulations at T0 were subjected to simulated shear stress for 24 hours at 900 RPM at room temperature and analysed for osmolality, pH, A280, RALS, SEC, and DLS. The results for osmolality, pH, A280, and RALS are shown in Table 85.

TABLE 85

| | Osmolality | | | | | | | | RALS | |
|---|---|---|---|---|---|---|---|---|---|---|
| | (mOsm/kg) | | pH | | Concentration | | | | T0 | |
| | T0 | | T0 | | T0 liquid | | Shear | | liquid | Shear |
| No. | liquid | Shear | liquid | Shear | A280 | μg/ml | A280 | μg/ml | 565 Volts | |
| E-CF | 30 | 32 | 7.54 | 7.57 | 0.034 | 22.19 | 0.076 | 35.25 | 0.764 | 4.909 |
| E1 | 348 | 349 | 7.52 | 7.48 | 0.036 | 23.5 | 0.054 | 28.72 | 1.416 | 6.637 |
| E2 | 347 | 348 | 7.53 | 7.51 | 0.037 | 24.15 | 0.053 | 28.07 | 1.337 | 4.472 |
| E3 | 348 | 352 | 7.54 | 7.55 | 0.037 | 24.15 | 0.064 | 33.29 | 1.656 | 5.153 |
| E4 | 332 | 336 | 7.53 | 7.6 | 0.034 | 22.19 | 0.064 | 32.64 | 0.942 | 5.162 |
| E5 | 340 | 345 | 7.52 | 7.51 | 0.037 | 24.15 | 0.066 | 32.64 | 1.401 | 5.862 |
| E6 | 384 | 387 | 7.57 | 7.65 | 0.034 | 22.19 | 0.083 | 35.25 | 1.339 | 9.998 |
| E7 | 353 | 358 | 7.58 | 7.59 | 0.04 | 26.11 | 0.076 | 37.86 | 1.404 | 5.643 |
| E8 | 381 | 385 | 7.57 | 7.63 | 0.033 | 21.54 | 0.077 | 38.51 | 1.255 | 9.905 |

As shown in Table 85, formulation E1 containing trehalose and E2 containing alanine and trehalose had the lowest concentrations after simulated shear stress suggesting that there were less light scattering particles which contributed or interfered with absorbance measurement at A280. Further, formulation E2 containing alanine and trehalose had better resistance to shear stress with the lowest RALS value. Formulation E6 containing arginine and trehalose, and E8 containing alanine, arginine, and trehalose were relatively more sensitive to shear stress compared to the other formulations as shown by higher RALS values.

The formulations were subjected to simulated shear stress for 24 hours at 900 RPM and monitored by SEC-HPLC for total recovery, fusion protein recovery, and percent impurities consisting of HMW and LMW. The results are shown in Tables 86 and 87. The % HMW increased for all liquid formulations after shear as monitored by SEC. The % LMW increased significantly for formulation E1 containing trehalose, and E8 containing alanine, arginine, and trehalose. Formulation E7 containing proline and trehalose also had the highest % main peak and E2 containing alanine and trehalose presented the highest % total recovery after shear, suggesting relatively less sensitivity to shear stress.

TABLE 86

| | T0 liquid | | | | | | |
|---|---|---|---|---|---|---|---|
| | % | % | % | HMW | Main | LMW | Total |
| No. | HMW | Main | LMW | AUC | AUC | AUC | AUC |
| E-CF | 2.0 | 89.6 | 8.3 | 219 | 9572 | 890 | 10681 |
| E1 | 3.6 | 95.7 | 0.7 | 336 | 8941 | 69 | 9346 |
| E2 | 1.8 | 97.2 | 1.0 | 164 | 8858 | 89 | 9111 |
| E3 | 1.9 | 97.3 | 0.8 | 173 | 8913 | 77 | 9163 |
| E4 | 1.7 | 97.7 | 0.6 | 152 | 8903 | 56 | 9111 |
| E5 | 1.8 | 97.3 | 0.9 | 163 | 8868 | 80 | 9111 |
| E6 | 1.6 | 96.6 | 1.8 | 148 | 8871 | 164 | 9183 |
| E7 | 1.8 | 96.8 | 1.4 | 182 | 9662 | 138 | 9982 |
| E8 | 1.2 | 98.3 | 0.5 | 111 | 9286 | 48 | 9445 |

TABLE 87

| | | | | | | | | % |
|---|---|---|---|---|---|---|---|---|
| | | | | | | | | total |
| | Simulated Shear Stress | | | | | | | re- |
| | % | % | % | HMW | Main | LMW | Total | cov- |
| No. | HMW | Main | LMW | AUC | AUC | AUC | AUC | ery |
| E- | 74.4 | 20.7 | 5.0 | 5213 | 1448 | 348 | 7009 | 65.6 |
| CF | | | | | | | | |
| E1 | 62.6 | 28.6 | 8.9 | 3751 | 1713 | 533 | 5997 | 64.2 |
| E2 | 63.8 | 33.5 | 2.7 | 4350 | 2288 | 185 | 6823 | 74.9 |
| E3 | 73.8 | 24.7 | 1.5 | 4814 | 1612 | 99 | 6525 | 71.2 |
| E4 | 73.6 | 24.8 | 1.6 | 4431 | 1492 | 94 | 6017 | 66.0 |
| E5 | 85.1 | 13.0 | 1.9 | 4701 | 716 | 108 | 5525 | 60.6 |
| E6 | 66.2 | 32.4 | 1.4 | 1936 | 948 | 42 | 2926 | 31.9 |
| E7 | 51.5 | 45.7 | 2.8 | 3218 | 2855 | 177 | 6250 | 62.6 |
| E8 | 59.4 | 32.5 | 8.0 | 2600 | 1424 | 352 | 4376 | 46.3 |

The formulations that were subjected to simulated shear stress were also analysed by DLS. The results are shown in Table 88. Formulation E1 (containing trehalose), E2 (containing alanine and trehalose), E3 (containing alanine and sucrose), and E7 (containing proline and trehalose) had relatively small particle size diameters after simulated shear stress with diameters around 1.2 nm.

TABLE 88

DLS after simulated
shear stress

| No. | P1 d (nm) | Vol % |
|---|---|---|
| E-CF | 78.16 | 100 |
| E1 | 1.309 | 100 |
| E2 | 1.276 | 100 |
| E3 | 1.202 | 100 |
| E4 | 129.3 | 100 |
| E5 | 25.31 | 95.1 |
| E6 | 344.3 | 67.5 |
| E7 | 1.156 | 100 |
| E8 | 254.6 | 56.7 |

In summary, the results of Example 5 demonstrated that arginine protected against thermal stress and alanine protected against shear stress. For example, formulation E8 (containing alanine, arginine, and trehalose) and formulation E2 (containing alanine and trehalose) stabilized SONE-IL12-ABD, and the liquid E2 and E8 formulation had the highest total percent recoveries on SEC after 14 days of incubation.

### Example 6: Stability Studies for Formulations with Varying Stabilizers

Formulations of SONE-IL12-ABD were prepared as shown in Table 89 using a SONE-IL12-ABD concentration of approximately 20 μg/ml.

TABLE 89

| No. | Formulation |
|---|---|
| F-LF | 50 mM alanine, 200 mM trehalose, 25 mM GlyGly, 10 μM |
| | DTPA, 0.02% PS20, pH 7.5 |
| F1 | 35 mM alanine, 15 mM arginine, 200 mM trehalose, 25 mM |
| | GlyGly, 10 μM DTPA, 0.02% PS20, pH 7.5 |
| F2 | 25 mM alanine, 25 mM arginine, 200 mM trehalose, 25 mM |
| | GlyGly, 10 μM DTPA, 0.02% PS20, pH 7.5 |
| F3 | 15 mM alanine, 35 mM arginine, 200 mM trehalose, 25 mM |
| | GlyGly, 10 μM DTPA, 0.02% PS20, pH 7.5 |
| F4 | 50 mM alanine, 150 mM trehalose, 50 mM mannitol, 25 mM |
| | GlyGly, 10 μM DTPA, 0.02% PS20, pH 7.5 |
| F5 | 50 mM alanine, 100 mM trehalose, 100 mM mannitol, 25 mM |
| | GlyGly, 10 μM DTPA, 0.02% PS20, pH 7.5 |
| F6 | 50 mM alanine, 50 mM trehalose, 150 mM mannitol, 25 mM |
| | GlyGly, 10 μM DTPA, 0.02% PS20, pH 7.5 |

The formulations shown in Table 89 were also lyophilized according to the lyophilization cycle shown in Table 66.

The liquid formulations were subjected to temperature ramping RALS and IF to find Tagg and Tm of each formulation. Tm and Tagg values are shown in Table 90. Tm and Tagg are similar across all formulations.

TABLE 90

| No. | Tm (° C.) | Tagg (° C.) |
|---|---|---|
| F-LF | 46 | 67 |
| F1 | 49 | 67 |
| F2 | 50 | 65 |
| F3 | 51 | 66 |
| F4 | 46 | 67 |
| F5 | 48 | 66 |
| F6 | 47 | 67 |

### 6.1. Thermal Stress Study

The liquid and lyophilized formulations were thermally stressed for 14 days at 40° C. and monitored for visual appearance, osmolality, pH, A280, RALS, Flowcam, SEC-HPLC, reducing and non-reducing SDS-page, and ELISA. The results for visual appearance are shown in Table 91, for osmolality, pH, and RALS in Tables 92 and 93, and for A280 in Tables 94 and 95.

As shown in Table 91, all liquid and reconstituted samples were clear, colorless, and had no visible particles. The T7 F4 vial, containing more trehalose than mannitol, had a defective stopper preventing it from lyophilizing, but T14 data on F4 was able to be measured. F6, containing more mannitol than trehalose, had a completely collapsed lyo cake at T0 and did not change at T14. The cakes for lyophilized formulations containing alanine and arginine demonstrated were improved compared to the cakes for lyophilized formulations containing mannitol and trehalose.

TABLE 91

| No. | T0, T7, T14 liquid | T0, T7, T14 lyophilized cake | T0, T7, T14 lyo reconstituted |
|---|---|---|---|
| F-LF | Clear, colorless, no visible particles | White, slightly pulling away from glass (no change from T0) | Clear, colorless, no visible particles |
| F1 | Clear, colorless, no visible particles | White, slightly pulling away from glass (no change from T0) | Clear, colorless, no visible particles |
| F2 | Clear, colorless, no visible particles | White, slightly pulling away from glass (no change from T0) | Clear, colorless, no visible particles |
| F3 | Clear, colorless, no visible particles | White, slightly pulling away from glass (no change from T0) | Clear, colorless, no visible particles |
| F4 | Clear, colorless, no visible particles | White, 20% pulling away from glass at T14 | Clear, colorless, no visible particles |
| F5 | Clear, colorless, no visible particles | White, 70% shrinkage/pulling away at T14 | Clear, colorless, no visible particles |
| F6 | Clear, colorless, no visible particles | White, completely collapsed cake (no change from T0) | Clear, colorless, no visible particles |

TABLE 92

| | Osmolality | | | | | | RALS | | |
|---|---|---|---|---|---|---|---|---|---|
| No. | (mOsm/kg) | | | pH | | | (565 Volts) | | |
| (liquid) | T0 | T7 | T14 | T0 | T7 | T14 | T0 | T7 | T14 |
| F-LF | 298 | 298 | 299 | 7.57 | 7.55 | 7.52 | 1.857 | 1.156 | 1.203 |
| F1 | 308 | 309 | 309 | 7.58 | 7.54 | 7.53 | 1.238 | 1.59 | 1.474 |
| F2 | 315 | 316 | 318 | 7.58 | 7.57 | 7.55 | 1.411 | 1.259 | 1.200 |
| F3 | 324 | 325 | 325 | 7.56 | 7.55 | 7.53 | 1.231 | 1.357 | 1.266 |
| F4 | 291 | 295 | 292 | 7.56 | 7.58 | 7.56 | 1.441 | 1.104 | 1.183 |
| F5 | 288 | 290 | 291 | 7.54 | 7.56 | 7.57 | 1.169 | 1.028 | 1.162 |
| F6 | 284 | 286 | 285 | 7.56 | 7.58 | 7.57 | 1.086 | 1.655 | 1.038 |

TABLE 93

| No. | Osmolality | | | | | | RALS | | |
|---|---|---|---|---|---|---|---|---|---|
| (lyoph- | (mOsm/kg) | | | pH | | | (565 Volts) | | |
| ilized) | T0 | T7 | T14 | T0 | T7 | T14 | T0 | T7 | T14 |
| F-LF | 295 | 310 | 311 | 7.59 | 7.55 | 7.53 | 1.555 | 1.645 | 1.77 |
| F1 | 308 | 315 | 323 | 7.59 | 7.56 | 7.53 | 1.703 | 1.231 | 1.43 |
| F2 | 315 | 318 | 356 | 7.61 | 7.55 | 7.54 | 1.421 | 1.288 | 1.391 |
| F3 | 322 | 332 | 338 | 7.58 | 7.54 | 7.52 | 2.091 | 1.278 | 1.263 |
| F4 | 289 | * | 304 | 7.58 | * | 7.57 | 1.314 | * | 1.208 |
| F5 | 291 | 298 | 303 | 7.55 | 7.58 | 7.56 | 1.14 | 1.193 | 1.088 |
| F6 | 287 | 295 | 300 | 7.55 | 7.57 | 7.58 | 1.036 | 1.01 | 1.232 |

* No data.

TABLE 94

| No. | T0 | | T7 | | T14 | | % Recovery |
|---|---|---|---|---|---|---|---|
| (liquid) | A280 | μg/ml | A280 | μg/ml | A280 | μg/ml | at T14 |
| F-LF | 0.04 | 26.11 | 0.033 | 21.54 | 0.034 | 22.19 | 85 |
| F1 | 0.039 | 25.46 | 0.039 | 25.46 | 0.039 | 25.46 | 100 |
| F2 | 0.045 | 29.37 | 0.032 | 20.89 | 0.03 | 19.58 | 67 |
| F3 | 0.04 | 26.11 | 0.035 | 22.85 | 0.032 | 20.89 | 80 |
| F4 | 0.043 | 28.07 | 0.032 | 20.89 | 0.033 | 21.54 | 77 |
| F5 | 0.039 | 25.46 | 0.029 | 18.93 | 0.033 | 21.54 | 85 |
| F6 | 0.043 | 28.07 | 0.035 | 22.85 | 0.034 | 22.19 | 79 |

TABLE 95

| No. | | | | | | | |
|---|---|---|---|---|---|---|---|
| (lyoph- | T0 | | T7 | | T14 | | % Recovery |
| ilized) | A280 | μg/ml | A280 | μg/ml | A280 | μg/ml | at T14 |
| F-LF | 0.037 | 24.15 | 0.042 | 27.42 | 0.039 | 25.46 | 105 |
| F1 | 0.042 | 27.42 | 0.041 | 26.76 | 0.036 | 23.50 | 86 |
| F2 | 0.033 | 21.54 | 0.038 | 24.80 | 0.034 | 22.19 | 103 |
| F3 | 0.037 | 24.15 | 0.037 | 24.15 | 0.035 | 22.85 | 95 |
| F4 | 0.040 | 26.11 | * | * | 0.030 | 19.58 | 75 |
| F5 | 0.033 | 21.54 | 0.034 | 22.19 | 0.034 | 22.19 | 103 |
| F6 | 0.035 | 22.85 | 0.04 | 26.11 | 0.039 | 25.46 | 111 |

* No data.

As shown in Table 93, osmolality increased more for F1, containing more alanine than arginine, and F2, containing equal amounts of alanine and arginine, in the T14 lyo samples.

As shown in Table 94, concentration decreased for all liquid formulations except for F1, containing more alanine than arginine.

The formulations were assessed for presence of sub-particulates by Flowcam after incubation for 7 and 14 days at 40° C. As shown in Table 96, sub-visible particle counts increased for all liquid formulations after incubation for 14 days, with the formulation F6, containing more mannitol than trehalose, increasing the most. As shown in Table 97, sub-visible particle counts increased for all lyophilized formulations after incubation for 14 days.

TABLE 96

| | T0 (liquid) | | | | T7 (liquid) | | | | T14 (liquid) | | | |
|---|---|---|---|---|---|---|---|---|---|---|---|---|
| | 2-10 | 10-25 | 25-50 | 50-100 | 2-10 | 10-25 | 25-50 | 50-100 | 2-10 | 10-25 | 25-50 | 50-100 |
| No. | um | um | um | um | um | um | um | um | um | um | um | um |
| F-LF | 520 | 124 | 0 | 31 | 1256 | 336 | 31 | 31 | 1164 | 152 | 31 | 0 |
| F1 | 676 | 60 | 0 | 0 | 676 | 124 | 0 | 0 | 856 | 184 | 60 | 0 |
| F2 | 460 | 124 | 60 | 0 | 736 | 184 | 31 | 31 | 1408 | 124 | 92 | 0 |
| F3 | 368 | 92 | 31 | 0 | 612 | 244 | 92 | 0 | 1040 | 92 | 92 | 0 |
| F4 | 612 | 216 | 31 | 0 | 704 | 276 | 31 | 31 | 1316 | 184 | 0 | 0 |
| F5 | 796 | 152 | 31 | 0 | 828 | 92 | 31 | 31 | 1992 | 308 | 31 | 0 |
| F6 | 400 | 124 | 31 | 0 | 1104 | 124 | 31 | 0 | 2480 | 244 | 0 | 0 |

TABLE 97

| | T0 (lyophilized) | | | | | T7 (lyophilized) | | | | | T14 (lyophilized) | | | | |
|---|---|---|---|---|---|---|---|---|---|---|---|---|---|---|---|
| | 2-10 | 10-25 | 25-50 | 50-100 | 100 | 2-10 | 10-25 | 25-50 | 50-100 | 100 | 2-10 | 10-25 | 25-50 | 50-100 | 100 |
| No. | um | um | um | um | um+ | um | um | um | um | um+ | um | um | um | um | um+ |
| F-LF | 520 | 92 | 60 | 0 | 0 | 1040 | 124 | 92 | 0 | 0 | 3708 | 612 | 152 | 0 | 0 |
| F1 | 920 | 400 | 60 | 0 | 0 | 1228 | 492 | 400 | 0 | 31 | 1348 | 428 | 124 | 31 | 31 |
| F2 | 1132 | 492 | 152 | 31 | 0 | 976 | 228 | 0 | 0 | 0 | 2268 | 584 | 276 | 0 | 0 |
| F3 | 2296 | 368 | 92 | 31 | 60 | 1104 | 644 | 124 | 0 | 0 | 1564 | 520 | 60 | 0 | 0 |
| F4 | 1592 | 368 | 124 | 0 | 31 | * | * | * | * | * | 2584 | 336 | 31 | 0 | 0 |
| F5 | 1748 | 276 | 216 | 60 | 31 | 460 | 124 | 92 | 31 | 0 | 1132 | 152 | 92 | 0 | 0 |
| F6 | 1224 | 244 | 184 | 0 | 0 | 676 | 124 | 31 | 0 | 0 | 2052 | 276 | 92 | 31 | 0 |

* No data.

The formulations were thermally stressed for 14 days at 40° C. and monitored by SEC-HPLC for total recovery, fusion protein recovery, and percent impurities consisting of HMW and LMW. The results are shown in Tables 98 to 103.

The liquid formulation F5, containing equal amounts of trehalose and mannitol, had the lowest LMW on SEC at T14. Liquid formulations containing arginine had very low HMW at T14. Percent main peak was highest for formulations containing arginine. The results also demonstrated that formulations containing arginine protected against thermal stress.

The % LMW decreased for all lyophilized formulations on SEC at T14. All lyophilized formulations had very low % HMW at T14. Percent main peak was relatively similar across all lyophilized formulations with F-LF and F5 (containing equal amounts of trehalose and mannitol) having the highest at 96%.

TABLE 98

| | T0 (liquid) | | | | | | |
|---|---|---|---|---|---|---|---|
| | % | % | % | HMW | Main | LMW | Total |
| No. | HMW | Main | LMW | AUC | AUC | AUC | AUC |
| F-LF | 2.9 | 89.0 | 4.8 | 296 | 9151 | 831 | 10278 |
| F1 | 2.1 | 79.9 | 18.0 | 224 | 8679 | 1955 | 10858 |
| F2 | 1.6 | 88.3 | 10.1 | 162 | 8879 | 1020 | 10061 |
| F3 | 1.4 | 89.4 | 9.2 | 144 | 9038 | 927 | 10109 |
| F4 | 1.0 | 91.2 | 7.8 | 101 | 9091 | 777 | 9969 |
| F5 | 1.2 | 91.5 | 7.3 | 121 | 9035 | 720 | 9876 |
| F6 | 1.1 | 89.8 | 9.1 | 113 | 9193 | 929 | 10235 |

TABLE 99

| | T7 (liquid) | | | | | | | |
|---|---|---|---|---|---|---|---|---|
| | | | | | | | | % |
| | | | | | | | | total |
| | | | | | | | | re- |
| | % | % | % | HMW | Main | LMW | Total | cov- |
| No. | HMW | Main | LMW | AUC | AUC | AUC | AUC | ery |
| F- | 9.7 | 86.9 | 2.8 | 829 | 7443 | 294 | 8566 | 83.3 |
| LF | | | | | | | | |
| F1 | 2.6 | 91.2 | 6.3 | 226 | 7981 | 549 | 8756 | 80.6 |
| F2 | 1.5 | 92.9 | 5.6 | 112 | 7086 | 426 | 7624 | 75.8 |
| F3 | 0.9 | 94.5 | 4.5 | 79 | 8159 | 391 | 8629 | 85.4 |
| F4 | 9.8 | 88.5 | 1.6 | 774 | 6977 | 129 | 7880 | 79.0 |
| F5 | 10.1 | 87.7 | 2.2 | 821 | 7121 | 178 | 8120 | 82.2 |
| F6 | 10.3 | 87.6 | 2.1 | 826 | 7030 | 167 | 8023 | 78.4 |

TABLE 100

| | T14 (liquid) | | | | | | | |
|---|---|---|---|---|---|---|---|---|
| | | | | | | | | % |
| | | | | | | | | Total |
| | | | | | | | | re- |
| | % | % | % | HMW | Main | LMW | Total | cov- |
| No. | HMW | Main | LMW | AUC | AUC | AUC | AUC | ery |
| F- | 13.8 | 82.2 | 3.9 | 1163 | 6918 | 331 | 8412 | 81.8 |
| LF | | | | | | | | |
| F1 | 6.0 | 87.3 | 6.7 | 538 | 7779 | 596 | 8913 | 82.1 |
| F2 | 3.4 | 92.0 | 4.5 | 290 | 7783 | 383 | 8456 | 84.0 |
| F3 | 2.0 | 93.2 | 4.8 | 167 | 7871 | 408 | 8446 | 83.5 |
| F4 | 15.3 | 82.9 | 1.8 | 1207 | 6517 | 139 | 7863 | 78.9 |
| F5 | 15.9 | 82.6 | 1.4 | 1244 | 6457 | 112 | 7813 | 79.1 |
| F6 | 15.3 | 82.0 | 2.7 | 1206 | 6449 | 214 | 7869 | 76.9 |

TABLE 101

| | T0 (lyophilized) | | | | | | | |
|---|---|---|---|---|---|---|---|---|
| | | | | | | | | % |
| | | | | | | | | total |
| | | | | | | | | re- |
| | % | % | % | HMW | Main | LMW | Total | cov- |
| No. | HMW | Main | LMW | AUC | AUC | AUC | AUC | ery |
| F- | 4.8 | 88.4 | 6.8 | 479 | 8798 | 680 | 9957 | 96.9 |
| LF | | | | | | | | |
| F1 | 2.4 | 90.5 | 7.2 | 229 | 8806 | 697 | 9732 | 89.6 |
| F2 | 1.8 | 92.1 | 6.1 | 167 | 8586 | 574 | 9327 | 92.7 |
| F3 | 1.6 | 91.6 | 6.9 | 145 | 8529 | 639 | 9313 | 92.1 |
| F4 | 1.5 | 90.6 | 7.9 | 140 | 8573 | 746 | 9459 | 94.9 |
| F5 | 1.3 | 92.8 | 5.9 | 112 | 7757 | 493 | 8362 | 84.7 |
| F6 | 1.3 | 90.4 | 8.3 | 123 | 8787 | 809 | 9719 | 95.0 |

TABLE 102

| | T7 (lyophilized) | | | | | | | |
|---|---|---|---|---|---|---|---|---|
| | | | | | | | | % |
| | | | | | | | | total |
| | | | | | | | | re- |
| | % | % | % | HMW | Main | LMW | Total | cov- |
| No. | HMW | Main | LMW | AUC | AUC | AUC | AUC | ery |
| F- | 1.3 | 95.8 | 2.9 | 125 | 9337 | 287 | 9749 | 97.9 |
| LF | | | | | | | | |
| F1 | 0.9 | 94.8 | 4.2 | 88 | 9056 | 404 | 9548 | 98.1 |
| F2 | 0.8 | 96.6 | 2.5 | 77 | 8841 | 229 | 9147 | 98.1 |
| F3 | 0.8 | 95.5 | 3.7 | 76 | 8839 | 343 | 9258 | 99.4 |
| F4 | * | * | * | * | * | * | * | * |
| F5 | 1.0 | 94.6 | 4.3 | 92 | 8699 | 399 | 9190 | 109.9 |
| F6 | 0.9 | 94.2 | 4.9 | 86 | 8987 | 465 | 9538 | 98.1 |

* No data.

TABLE 103

| | T14 (lyophilized) | | | | | | | |
|---|---|---|---|---|---|---|---|---|
| | % | % | % | HMW | Main | LMW | Total | % Total |
| No. | HMW | Main | LMW | AUC | AUC | AUC | AUC | recovery |
| F-LF | 0.8 | 96.3 | 2.9 | 78 | 9361 | 278 | 9717 | 97.6 |
| F1 | 1.2 | 95.8 | 3.0 | 112 | 8989 | 279 | 9380 | 96.4 |
| F2 | 0.8 | 94.8 | 4.4 | 81 | 9492 | 446 | 10019 | 107.4 |
| F3 | 0.8 | 94.4 | 4.8 | 80 | 8952 | 451 | 9403 | 101.0 |
| F4 | 0.9 | 96.2 | 2.9 | 89 | 9121 | 275 | 9485 | 100.3 |
| F5 | 0.9 | 96.4 | 2.7 | 82 | 9240 | 259 | 9581 | 114.6 |
| F6 | 0.9 | 94.5 | 4.6 | 90 | 9027 | 435 | 9552 | 98.3 |

The liquid and lyophilized formulations F-LF, F1, F2, and F3 that were thermally stressed for 14 days at 40° C. were also analyzed by reducing and non-reducing SDS-PAGE. Qualitative analysis by silver staining indicated that formulation F-LF and the formulations containing mannitol demonstrated the lowest impurities.

Liquid and lyophilized formulations F-LF, F4, F5, and F6 were also analysed by ELISA. The results are shown in Table 104.

TABLE 104

| No. | Timepoint | Avg [C] μg/mL |
|---|---|---|
| F-LF (liquid) | T0 | 19.69 |
| | T14 | 6.28 |
| F-LF (lyophilized) | T0 | 22.6 |
| | T14 | 30.63 |
| F4 (liquid) | T0 | 19.68 |
| | T7 | 21.77 |
| | T14 | 5.66 |
| F4 (lyophilized) | T0 | 21.66 |
| | T7 | 19.66 |
| | T14 | 22.05 |
| F5 (liquid) | T0 | 6.19 |
| | T14 | 22.55 |
| F5 (lyophilized) | T0 | 17.55 |
| | T14 | 26.74 |
| F6 (liquid) | T0 | 6.99 |
| | T14 | 19.57 |
| F6 (lyophilized) | T0 | 19.66 |
| | T14 | 21.77 |

### 6.2. Freeze-Thaw Study

The formulations at T0 were subjected to five freeze-thaws and then analyzed for osmolality, pH, RALS, A280, Flowcam, and SEC-HPLC. The results for osmolality, pH, A280, and RALS are shown in Table 105.

TABLE 105

| | Osmolality | | | | | | | | | RALS | |
|---|---|---|---|---|---|---|---|---|---|---|---|
| | (mOsm/kg) | | pH | | Concentration | | | | | (565 Volts) | |
| | T0 | Freeze- | T0 | Freeze- | T0 liquid | | Freeze-thaw | | % | T0 | Freeze- |
| No. | liquid | thaw | liquid | thaw | A280 | μg/ml | A280 | μg/ml | recovery | liquid | thaw |
| F-LF | 298 | 295 | 7.57 | 7.59 | 0.04 | 26.11 | 0.029 | 18.93 | 73 | 1.857 | 1.132 |
| F1 | 308 | 307 | 7.58 | 7.61 | 0.039 | 25.46 | 0.03 | 19.58 | 77 | 1.238 | 1.233 |
| F2 | 315 | 318 | 7.58 | 7.62 | 0.045 | 29.37 | 0.038 | 24.8 | 84 | 1.411 | 1.380 |
| F3 | 324 | 324 | 7.56 | 7.6 | 0.04 | 26.11 | 0.032 | 20.89 | 80 | 1.231 | 1.223 |
| F4 | 291 | 292 | 7.56 | 7.57 | 0.043 | 28.07 | 0.037 | 24.15 | 86 | 1.441 | 1.238 |
| F5 | 288 | 289 | 7.54 | 7.57 | 0.039 | 25.46 | 0.037 | 24.15 | 95 | 1.169 | 1.133 |
| F6 | 284 | 283 | 7.56 | 7.57 | 0.043 | 28.07 | 0.036 | 23.5 | 84 | 1.086 | 0.921 |

The formulations were assessed for presence of sub-particulates by Flowcam after five freeze-thaw cycles. The results are shown in Table 106. Sub-visible particles in the 2-10 μm range increased for all formulations.

TABLE 106

| | T0 liquid | | | | | Freeze-thaw | | | | |
|---|---|---|---|---|---|---|---|---|---|---|
| | 2-10 | 10-25 | 25-50 | 50-100 | 100 | 2-10 | 10-25 | 25-50 | 50-100 | 100 |
| No. | um | um | um | um | um+ | um | um | um | um | um+ |
| F-LF | 520 | 124 | 0 | 31 | 0 | 1196 | 152 | 31 | 0 | 0 |
| F1 | 676 | 60 | 0 | 0 | 0 | 764 | 232 | 100 | 0 | 0 |
| F2 | 460 | 124 | 60 | 0 | 0 | 1204 | 516 | 44 | 0 | 0 |
| F3 | 368 | 92 | 31 | 0 | 0 | 1224 | 428 | 92 | 0 | 0 |
| F4 | 612 | 216 | 31 | 0 | 0 | 1164 | 152 | 0 | 0 | 0 |
| F5 | 796 | 152 | 31 | 0 | 0 | 980 | 124 | 0 | 0 | 0 |
| F6 | 400 | 124 | 31 | 0 | 0 | 1828 | 496 | 0 | 0 | 0 |

The formulations were subjected to five freeze-thaws and monitored by SEC-HPLC for total recovery, fusion protein recovery, and percent impurities consisting of HMW and LMW. The results are shown in Tables 107 and 108. The % LMW significantly decreased on SEC for F1 after freeze-thaw. The % Main after freeze-thaw was consistent around 93% for all formulations. F-LF containing alanine and trehalose, and F4 containing more trehalose than mannitol, had the highest percent total recovery after freeze-thaw at 90%.

TABLE 107

| | T0 liquid | | | | | | |
|---|---|---|---|---|---|---|---|
| | % | % | % | HMW | Main | LMW | Total |
| No. | HMW | Main | LMW | AUC | AUC | AUC | AUC |
| F-LF | 2.9 | 89.0 | 4.8 | 296 | 9151 | 831 | 10278 |
| F1 | 2.1 | 79.9 | 18.0 | 224 | 8679 | 1955 | 10858 |
| F2 | 1.6 | 88.3 | 10.1 | 162 | 8879 | 1020 | 10061 |
| F3 | 1.4 | 89.4 | 9.2 | 144 | 9038 | 927 | 10109 |
| F4 | 1.0 | 91.2 | 7.8 | 101 | 9091 | 777 | 9969 |
| F5 | 1.2 | 91.5 | 7.3 | 121 | 9035 | 720 | 9876 |
| F6 | 1.1 | 89.8 | 9.1 | 113 | 9193 | 929 | 10235 |

TABLE 108

| | Freeze-thaw | | | | | | | |
|---|---|---|---|---|---|---|---|---|
| | | | | | | | | % |
| | | | | | | | | total |
| | | | | | | | | re- |
| | % | % | % | HMW | Main | LMW | Total | cov- |
| No. | HMW | Main | LMW | AUC | AUC | AUC | AUC | ery |
| F- | 1.8 | 92.1 | 6.1 | 165 | 8577 | 572 | 9314 | 90.6 |
| LF | | | | | | | | |
| F1 | 1.4 | 93.9 | 4.7 | 130 | 8716 | 438 | 9284 | 85.5 |
| F2 | 1.1 | 93.2 | 5.7 | 103 | 8415 | 514 | 9032 | 89.8 |
| F3 | 1.1 | 93.1 | 5.8 | 102 | 8387 | 520 | 9009 | 89.1 |
| F4 | 1.1 | 94.0 | 5.0 | 95 | 8507 | 448 | 9050 | 90.8 |
| F5 | 0.9 | 93.5 | 5.6 | 81 | 8269 | 493 | 8843 | 89.5 |
| F6 | 0.9 | 93.1 | 6.0 | 86 | 8520 | 548 | 9154 | 89.4 |

### 6.3. Simulated Shear Stress Study

The formulations at T0 were subjected to simulated shear stress for 24 hours at 900 RPM at room temperature and analysed for osmolality, pH, A280, RALS, SEC, and DLS. The results for osmolality, pH, A280, and RALS are shown in Tables 109 and 110.

TABLE 109

| | Osmolality | | | | | | | | | |
|---|---|---|---|---|---|---|---|---|---|---|
| | (mOsm/kg) | | pH | | Concentration | | | | | |
| | T0 | | T0 | | T0 liquid | | Shear | | | % |
| No. | liquid | Shear | liquid | Shear | A280 | μg/ml | A280 | A330 | μg/ml | Recovery |
| F-LF | 298 | 297 | 7.57 | 7.56 | 0.04 | 26.11 | 0.052 | 0.011 | 26.76 | 102 |
| F1 | 308 | 307 | 7.58 | 7.57 | 0.039 | 25.46 | 0.053 | 0.011 | 27.42 | 108 |
| F2 | 315 | 317 | 7.58 | 7.59 | 0.045 | 29.37 | 0.08 | 0.031 | 31.98 | 109 |
| F3 | 324 | 325 | 7.56 | 7.57 | 0.04 | 26.11 | 0.071 | 0.019 | 33.94 | 130 |
| F4 | 291 | 294 | 7.56 | 7.57 | 0.043 | 28.07 | 0.048 | 0.007 | 26.76 | 95 |
| F5 | 288 | 289 | 7.54 | 7.56 | 0.039 | 25.46 | 0.121 | 0.068 | 34.6 | 136 |
| F6 | 284 | 284 | 7.56 | 7.56 | 0.043 | 28.07 | 0.064 | 0.015 | 31.98 | 114 |

TABLE 110

RALS

| | |
|---|---|
| T0 liquid | Shear |

| | |
|---|---|
| No. | 565 Volts |

| | | |
|---|---|---|
| F-LF | 1.857 | 4.882 |
| F1 | 1.238 | 5.180 |
| F2 | 1.411 | 8.257 |
| F3 | 1.231 | 9.733 |
| F4 | 1.441 | 4.482 |
| F5 | 1.169 | 5.503 |
| F6 | 1.086 | 4.556 |

As shown in Table 109, formulation concentration increased slightly for all formulations after simulated shear stress. As shown in Table 110, RALS values increased for all formulations after simulated shear stress. Formulation F2 (containing more alanine than arginine), and F3 (containing more arginine than alanine), had very high RALS values indicating these formulations are sensitive to shear stress.

The formulations were subjected to simulated shear stress for 24 hours at 900 RPM and monitored by SEC-HPLC for total recovery, fusion protein recovery, and percent impurities consisting of HMW and LMW. The results are shown in Tables 111 and 112. The % LMW was lowest for F1 containing more alanine than arginine, and F3 containing more arginine than alanine, on SEC after simulated shear stress. The % HMW increased for all formulations after shear, and F-LF containing alanine and trehalose had the highest % main peak. F-LF containing alanine and trehalose, F4 containing more trehalose than mannitol, and F6 containing more mannitol than trehalose, had the highest % total recovery after simulated shear stress.

TABLE 111

| | T0 liquid | | | | | | |
|---|---|---|---|---|---|---|---|
| | % | % | % | HMW | Main | LMW | Total |
| No. | HMW | Main | LMW | AUC | AUC | AUC | AUC |
| F-LF | 2.9 | 89.0 | 4.8 | 296 | 9151 | 831 | 10278 |
| F1 | 2.1 | 79.9 | 18.0 | 224 | 8679 | 1955 | 10858 |
| F2 | 1.6 | 88.3 | 10.1 | 162 | 8879 | 1020 | 10061 |
| F3 | 1.4 | 89.4 | 9.2 | 144 | 9038 | 927 | 10109 |
| F4 | 1.0 | 91.2 | 7.8 | 101 | 9091 | 777 | 9969 |
| F5 | 1.2 | 91.5 | 7.3 | 121 | 9035 | 720 | 9876 |
| F6 | 1.1 | 89.8 | 9.1 | 113 | 9193 | 929 | 10235 |

TABLE 112

| | | | | | | | | % |
|---|---|---|---|---|---|---|---|---|
| | | | | | | | | total |
| | Simulated Shear Stress | | | | | | | re- |
| | % | % | % | HMW | Main | LMW | Total | cov- |
| No. | HMW | Main | LMW | AUC | AUC | AUC | AUC | ery |
| F- | 33.7 | 62.4 | 3.8 | 1616 | 2991 | 182 | 4789 | 46.6 |
| LF | | | | | | | | |
| F1 | 61.4 | 37.8 | 0.8 | 2180 | 1341 | 28 | 3549 | 32.7 |
| F2 | 53.3 | 34.8 | 12.0 | 2158 | 1407 | 484 | 4049 | 40.2 |
| F3 | 67.5 | 31.7 | 0.9 | 1774 | 833 | 22 | 2629 | 26.0 |
| F4 | 55.0 | 41.9 | 3.1 | 3062 | 2331 | 173 | 5566 | 55.8 |
| F5 | 79.8 | 19.1 | 1.1 | 2820 | 675 | 38 | 3533 | 35.8 |
| F6 | 42.0 | 56.7 | 1.3 | 2140 | 2886 | 65 | 5091 | 49.7 |

The formulations that were subjected to simulated shear stress were also analysed by DLS. The results are shown in Table 113. Formulation F-LF containing alanine and trehalose, and F4 containing more trehalose than mannitol, had particle diameters around 1.2 and 1.5 nm for 100% of its volume on DLS, suggesting that these formulations protect SONE-IL12-ABD against shear stress.

TABLE 113

DLS after simulated
shear stress

| No. | P1 d (nm) | Vol % |
|---|---|---|
| F-LF | 1.159 | 100 |
| F1 | 64.39 | 70.1 |
| F2 | 52.2 | 83.4 |
| F3 | 208.6 | 73.4 |
| F4 | 1.475 | 100 |
| F5 | 75.80 | 66.2 |
| F6 | 42.87 | 84.4 |

In summary, the results of Example 6 demonstrated that arginine protected against thermal stress and alanine protected against shear stress. The results further demonstrated that formulations containing the combination of alanine and arginine were more stable against thermal stress than against the shear stress. Additionally, the results demonstrated that the appearance of the lyophilized cake for formulation F-LF was relatively improved compared to formulations containing a combination of trehalose and mannitol.

### Example 7: Lyophilization Development

The formulation of SONE-IL12-ABD shown in Table 114 using a SONE-IL12-ABD concentration of 20 μg/ml was prepared.

TABLE 114

| No. | Formulation |
|---|---|
| LF | 50 mM alanine, 200 mM trehalose, 25 mM GlyGly, 10 μM |
| | DTPA, 0.02% PS20, pH 7.5 |

Formulation LF was studied for its thermal properties using modulated differential scanning calorimetry (MDSC). The MDSC conditions and results are shown in Table 115.

TABLE 115

| | Super | Freezing | Tg′ | Melting |
|---|---|---|---|---|
| Events | cooling (° C.) | (° C.) | (° C.) | (° C.) |
| Heat flow | −19.2 | −14.22 | −36.15, −28.61 | 0.55 |
| Reverse heat flow | NA | NA | −32.72, −28.00 | NA |

The formulation shown in Table 114 was also lyophilized according to the lyophilization cycle shown in Table 116.

TABLE 116

| | | | Temperature | Hold | |
|---|---|---|---|---|---|
| Step | Temperature | | Ramp (° C./min) | in hrs | Pressure |
| Loading | ambient | | N/A | N/A | NA |
| Freeze | −50° | C. | 1° C./min. | ~4.5 | NA |
| Primary Drying | −30° | C. | 1° C./min | ~40 | 70 mTorr |
| Secondary Drying | +20° | C. | 1° C./min | ~4.0 | 70 mTorr |

The liquid formulation was subjected to temperature ramping RALS and IF to find Tagg and Tm of the formulation. Tm and Tagg values are shown in Table 117.

TABLE 117

| No. | Tm (° C.) | Tagg (° C.) |
|---|---|---|
| LF | 48 | 67 |

### 7.1. Thermal Stress Study

The liquid and lyophilized formulations were thermally stressed for 14 days at 40° C. and monitored for visual appearance, osmolality, pH, A280, RALS, Flowcam, SEC-HPLC, reducing and non-reducing SDS-page, and ELISA. The results for visual appearance are shown in Table 118, for osmolality, pH, and RALS in Tables 119 and 120, and for A280 in Tables 121 and 122.

As shown in Table 118, liquid and reconstituted samples were clear, colorless, and had no visible particles. Lyophilized cakes at T0, T7, and T14 were all white and slightly pulling away from the glass. Some T0 lyophilized cakes were cracked, while others were not.

TABLE 118

| | T0 & T7 liquid | T7 lyophilized cake | T0 & T7 lyo reconstituted |
|---|---|---|---|
| LF | Clear, colorless, no visible particles | White, slightly pulling away from glass (no change from T0) | Clear, colorless, no visible particles |
| | T14 liquid | T14 lyophilized cake | T14 lyo reconstituted |
| LF | Clear, colorless, no visible particles | White, slightly pulling away from glass (no change from T0) | Clear, colorless, no visible particles |

TABLE 119

| | Osmolality | | | | | | RALS | | |
|---|---|---|---|---|---|---|---|---|---|
| No. | (mOsm/kg) | | | pH | | | (565 Volts) | | |
| (liquid) | T0 | T7 | T14 | T0 | T7 | T14 | T0 | T7 | T14 |
| LF | 296 | 294 | 294 | 7.54 | 7.52 | 7.55 | 1.732 | 1.367 | 1.265 |

TABLE 120

| No. | Osmolality | | | | | | RALS | | |
|---|---|---|---|---|---|---|---|---|---|
| (lyoph- | (mOsm/kg) | | | pH | | | (565 Volts) | | |
| ilized) | T0 | T7 | T14 | T0 | T7 | T14 | T0 | T7 | T14 |
| LF | 296 | 293 | 295 | 7.54 | 7.53 | 7.54 | 1.281 | 1.524 | 1.435 |

TABLE 121

| No. | T0 | | T7 | | T14 | | % Recovery |
|---|---|---|---|---|---|---|---|
| (liquid) | A280 | μg/ml | A280 | μg/ml | A280 | μg/ml | at T14 |
| LF | 0.044 | 28.7 | 0.036 | 23.5 | 0.034 | 22.2 | 77 |

TABLE 122

| No. | T0 | | T7 | | T14 | | % Recovery |
|---|---|---|---|---|---|---|---|
| (lyophilized) | A280 | μg/ml | A280 | μg/ml | A280 | μg/ml | at T14 |
| LF | 0.037 | 24.15 | 0.035 | 22.8 | 0.037 | 24.15 | 100 |

The formulations were assessed for presence of sub-particulates by Flowcam after incubation for 7 and 14 days at 40° C. As shown in Table 123, sub-visible particle counts increased for the liquid formulation after incubation for 14 days. As shown in Table 124, sub-visible particle counts also increased for the lyophilized formulation after incubation for 14 days.

TABLE 123

| | T0 (liquid) | | | | T7 (liquid) | | | | T14 (liquid) | | | |
|---|---|---|---|---|---|---|---|---|---|---|---|---|
| | 2-10 | 10-25 | 25-50 | 50-100 | 2-10 | 10-25 | 25-50 | 50-100 | 2-10 | 10-25 | 25-50 | 50-100 |
| No. | um | um | um | um | um | um | um | um | um | um | um | um |
| LF | 92 | 8 | 0 | 0 | 257 | 47 | 16 | 0 | 369 | 46 | 15 | 0 |

TABLE 124

| | T0 (lyophilized) | | | | T7 (lyophilized) | | | | T14 (lyophilized) | | | |
|---|---|---|---|---|---|---|---|---|---|---|---|---|
| | 2-10 | 10-25 | 25-50 | 50-100 | 2-10 | 10-25 | 25-50 | 50-100 | 2-10 | 10-25 | 25-50 | 50-100 |
| No. | um | um | um | um | um | um | um | um | um | um | um | um |
| LF | 291 | 38 | 31 | 8 | 250 | 54 | 23 | 8 | 260 | 23 | 8 | 0 |

The formulations were thermally stressed for 14 days at 40° C. and monitored by SEC-HPLC for total recovery, fusion protein recovery, and percent impurities consisting of HMW and LMW. The results are shown in Tables 125 to 130. The % HMW increased significantly, % LMW decreased, and % Main peak decreased at T14 for the liquid formulation. The % HMW slightly increased, % Main peak did not change significantly, and % LMW did not change significantly for the lyophilized formulation at T14. There was 100% recovery at T14 for the lyophilized sample.

TABLE 125

| | T0 (liquid) | | | | | | |
|---|---|---|---|---|---|---|---|
| No. | % HMW | % Main | % LMW | HMW AUC | Main AUC | LMW AUC | Total AUC |
| LF | 1.3 | 94.6 | 4.1 | 136 | 9917 | 429 | 10482 |

TABLE 126

| | T7 (liquid) | | | | | | | |
|---|---|---|---|---|---|---|---|---|
| | | | | HMW | Main | LMW | Total | % total |
| No. | % HMW | % Main | % LMW | AUC | AUC | AUC | AUC | recovery |
| LF | 12.8 | 84.0 | 3.2 | 1094 | 7165 | 276 | 8534 | 81.4 |

TABLE 127

| | T14 (liquid) | | | | | | | |
|---|---|---|---|---|---|---|---|---|
| | | | | HMW | Main | LMW | Total | % Total |
| No. | % HMW | % Main | % LMW | AUC | AUC | AUC | AUC | recovery |
| LF | 17.4 | 80.7 | 2.2 | 1410 | 6507 | 184 | 8101 | 77.3 |

TABLE 128

| | T0 (lyophilized) | | | | | | | |
|---|---|---|---|---|---|---|---|---|
| | | | | HMW | Main | LMW | Total | % total |
| No. | % HMW | % Main | % LMW | AUC | AUC | AUC | AUC | recovery |
| LF | 1.7 | 95.5 | 2.7 | 137 | 9093 | 263 | 9493 | 90.6 |

TABLE 129

| | T7 (lyophilized) | | | | | | | |
|---|---|---|---|---|---|---|---|---|
| | | | | HMW | Main | LMW | Total | % total |
| No. | % HMW | % Main | % LMW | AUC | AUC | AUC | AUC | recovery |
| LF | 1.9 | 94.7 | 3.4 | 187 | 9226 | 329 | 9742 | 102.6 |

TABLE 130

| | T14 (lyophilized) | | | | | | | |
|---|---|---|---|---|---|---|---|---|
| | | | | HMW | Main | LMW | Total | % Total |
| No. | % HMW | % Main | % LMW | AUC | AUC | AUC | AUC | recovery |
| LF | 2.2 | 95.3 | 2.3 | 236 | 9268 | 220 | 9723 | 102.4 |

The liquid and lyophilized formulations LF that were thermally stressed for 14 days at 40° C. were also analyzed by reducing and non-reducing SDS-PAGE. Qualitative analysis by silver staining showed no significant differences between liquid and lyophilized formulations.

Liquid and lyophilized formulations LF were also analysed by ELISA. The results are shown in Table 131.

TABLE 131

| No. | Timepoint | [C] in μg/mL |
|---|---|---|
| LF (liquid) | T0 | 16.97 |
| | T7 | 5.09 |
| | T14 | 3.73 |
| LF (lyophilized) | T0 | 15.49 |
| | T7 | 13.48 |
| | T14 | 12.15 |

### 7.2. Freeze-Thaw Study

The formulation LF at T0 was subjected to five freeze-thaws and then analyzed for osmolality, pH, RALS, A280, Flowcam, and SEC-HPLC. The results for osmolality, pH, A280, and RALS are shown in Table 132.

TABLE 132

| | Osmolality | | | | Concentration | | | | | RALS | |
|---|---|---|---|---|---|---|---|---|---|---|---|
| | (mOsm/kg) | | pH | | | | Freeze- | | | (565 Volts) | |
| | T0 | Freeze- | T0 | Freeze- | T0 liquid | | thaw | | % | T0 | Freeze- |
| No. | liquid | thaw | liquid | thaw | A280 | μg/ml | A280 | μg/ml | recovery | liquid | thaw |
| LF | 296 | 296 | 7.54 | 7.56 | 0.044 | 28.7 | 0.04 | 26.11 | 90.9 | 1.732 | 1.525 |

The formulation LF was assessed for presence of sub-particulates by Flowcam after five freeze-thaw cycles. The results are shown in Table 133. Sub-visible particles increased after freeze-thaw.

TABLE 133

| | T0 liquid | | | | | Freeze-thaw | | | | |
|---|---|---|---|---|---|---|---|---|---|---|
| | 2-10 | 10-25 | 25-50 | 50-100 | 100 | 2-10 | 10-25 | 25-50 | 50-100 | 100 |
| No. | um | um | um | um | um+ | um | um | um | um | um+ |
| LF | 92 | 8 | 0 | 0 | 0 | 506 | 230 | 15 | 0 | 0 |

The formulation LF was subjected to five freeze-thaws and monitored by SEC-HPLC for total recovery, fusion protein recovery, and percent impurities consisting of HMW and LMW. The results are shown in Tables 134 and 135. After freeze-thaw % LMW decreased by almost half and % HMW slightly increased.

TABLE 134

| | T0 liquid | | | | | | |
|---|---|---|---|---|---|---|---|
| No. | % HMW | % Main | % LMW | HMW AUC | Main AUC | LMW AUC | Total AUC |
| LF | 1.3 | 94.6 | 4.1 | 136 | 9917 | 429 | 10482 |

TABLE 135

| | Freeze-thaw | | | | | | | |
|---|---|---|---|---|---|---|---|---|
| | | | | HMW | Main | LMW | Total | % total |
| No. | % HMW | % Main | % LMW | AUC | AUC | AUC | AUC | recovery |
| LF | 2.0 | 95.2 | 2.8 | 179 | 8528 | 252 | 8959 | 85.5 |

### 7.3. Simulated Shear Stress Study

The formulation LF at T0 was subjected to simulated shear stress for 24 hours at 900 RPM at room temperature and analysed for osmolality, pH, A280, RALS, SEC, and DLS. The results for osmolality, pH, A280, and RALS are shown in Tables 136 and 137.

TABLE 136

| | Osmolality | | | | | | | | | |
|---|---|---|---|---|---|---|---|---|---|---|
| | (mOsm/kg) | | pH | | Concentration | | | | | |
| | T0 | | T0 | | T0 liquid | | Shear | | | % |
| No. | liquid | Shear | liquid | Shear | A280 | μg/ml | A280 | A330 | μg/ml | Recovery |
| LF | 296 | 295 | 7.54 | 7.57 | 0.044 | 28.7 | 0.054 | 0.005 | 31.98 | 111.4 |

TABLE 137

RALS

| | |
|---|---|
| T0 liquid | Shear |

| | |
|---|---|
| No. | 565 Volts |

| | | |
|---|---|---|
| F-LF | 1.732 | 4.245 |

The formulation LF was subjected to simulated shear stress for 24 hours at 900 RPM and monitored by SEC-HPLC for total recovery, fusion protein recovery, and percent impurities consisting of HMW and LMW. The results are shown in Tables 138 and 139. The % HMW significantly increased, % LMW decreased, and % Main peak decreased on SEC after simulated shear stress.

TABLE 138

| | T0 liquid | | | | | | |
|---|---|---|---|---|---|---|---|
| No. | % HMW | % Main | % LMW | HMW AUC | Main AUC | LMW AUC | Total AUC |
| LF | 1.3 | 94.6 | 4.1 | 136 | 9917 | 429 | 10482 |

TABLE 139

| | Simulated Shear Stress | | | | | | | |
|---|---|---|---|---|---|---|---|---|
| | | | | HMW | Main | LMW | Total | % total |
| No. | % HMW | % Main | % LMW | AUC | AUC | AUC | AUC | recovery |
| LF | 39.1 | 60.4 | 0.5 | 2081 | 3213 | 29 | 5323 | 50.8 |

The formulation LF that was subjected to simulated shear stress was also analysed by DLS. The results are shown in Table 140.

TABLE 140

DLS after simulated
shear stress

| No. | P3 d | Vol % |
|---|---|---|
| F-LF | 37.69 | 82.2 |

In summary, the results of Example 7 demonstrated that formulation LF in its lyophilized dosage form is stable for 14 days at 40° C., retaining full activity by ELISA and having no increase in HMW or LMW species.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, the group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventors expect skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A stable liquid pharmaceutical formulation, comprising:
about 10 μg/mL to about 50 μg/mL fusion protein of SEQ ID NO: 11;
about 25 mM to about 100 mM alanine;
about 100 mM to about 400 mM trehalose;
about 10 mM to about 50 mM glycylglycine;
about 0.01% to about 0.04% polysorbate 20 (v/v); and
about 5 μM to about 20 μM diethylenetriaminepentaacetic acid (DTPA),
wherein the formulation has a pH of about 6.8 to about 8.0.

2. The liquid formulation of claim 1, wherein the formulation comprises the fusion protein of SEQ ID NO: 11 in an amount of about 10 μg/mL to about 30 μg/mL, about 15 μg/mL to about 25 μg/mL, about 10 μg/mL to about 15 μg/mL, about 15 μg/mL to about 20 μg/mL, about 20 μg/mL to about 25 μg/mL, about 25 μg/mL to about 30 μg/mL, about 30 μg/mL to about 35 μg/mL, about 35 μg/mL to about 40 μg/mL, about 40 μg/mL to about 45 μg/mL, about 45 μg/mL to about 50 μg/mL, about 10 μg/mL, about 15 μg/mL, about 20 μg/mL, about 25 μg/mL, about 30 μg/mL, about 35 μg/mL, about 40 μg/mL, about 45 μg/mL, and/or about 50 μg/mL, preferably wherein the formulation comprises the fusion protein of SEQ ID NO: 11 in an amount of about 10 μg/mL to about 30 μg/mL.

3. The liquid formulation of claim 1, wherein the formulation comprises about 20 μg/ml fusion protein of SEQ ID NO: 11.

4. The liquid formulation of any one of claims 1 to 3, wherein the formulation comprises about 25 mM to about 75 mM alanine, about 40 mM to about 60 mM alanine, about 25 mM to about 30 mM alanine, about 30 mM to about 40 mM alanine, about 40 mM to about 50 mM alanine, about 50 mM to about 60 mM alanine, about 60 mM to about 70 mM alanine, about 70 mM to about 80 mM alanine, about 80 mM to about 90 mM alanine, about 90 mM to about 100 mM alanine, about 25 mM alanine, about 30 mM alanine, about 40 mM alanine, about 50 mM alanine, about 60 mM alanine, about 70 mM alanine, about 80 mM alanine, about 90 mM alanine, and/or about 100 mM alanine, preferably wherein the formulation comprises about 50 mM alanine.

5. The liquid formulation of any one of claims 1 to 4, wherein the formulation comprises about 100 mM to about 300 mM trehalose, about 150 mM to about 250 mM trehalose, about 100 mM to about 150 mM trehalose, about 150 mM to about 200 mM trehalose, about 200 mM to about 250 mM trehalose, about 250 mM to about 300 mM trehalose, about 300 mM to about 350 mM trehalose, about 350 mM to about 400 mM trehalose, about 100 mM trehalose, about 150 mM trehalose, about 200 mM trehalose, about 250 mM trehalose, about 300 mM trehalose, about 350 mM trehalose, and/or about 400 mM trehalose, preferably wherein the formulation comprises about 200 mM trehalose.

6. The liquid formulation of any one of claims 1 to 5, wherein the formulation comprises about 10 mM to about 40 mM glycylglycine, about 10 mM to about 30 mM glycylglycine, about 10 mM to about 15 mM glycylglycine, about 15 mM to about 20 mM glycylglycine, about 20 mM to about 25 mM glycylglycine, about 25 mM to about 30 mM glycylglycine, about 35 mM to about 40 mM glycylglycine, about 40 mM to about 45 mM glycylglycine, about 45 mM to about 50 mM glycylglycine, about 10 mM glycylglycine, about 15 mM glycylglycine, about 20 mM glycylglycine, about 25 mM glycylglycine, about 30 mM glycylglycine, about 35 mM glycylglycine, about 40 mM glycylglycine, about 45 mM glycylglycine, and/or about 50 mM glycylglycine, preferably wherein the formulation comprises about 25 mM glycylglycine.

7. The liquid formulation of any one of claims 1 to 6, wherein the formulation comprises about 0.01% to about 0.03% polysorbate 20 (v/v), about 0.01% to about 0.02% polysorbate 20 (v/v), about 0.02% to about 0.03% polysorbate 20 (v/v), about 0.03% to about 0.04% polysorbate 20 (v/v), about 0.01% polysorbate 20 (v/v), about 0.02% polysorbate 20 (v/v), about 0.03% polysorbate 20 (v/v), and/or about 0.04% polysorbate 20 (v/v), preferably wherein the formulation comprises about 0.02% polysorbate 20 (v/v).

8. The liquid formulation of any one of claims 1 to 7, wherein the formulation comprises about 5 μM to about 15 μM DTPA, about 5 μM to about 10 μM DTPA, about 10 μM to about 15 M DTPA, about 15 μM to about 20 μM DTPA, about 5 μM DTPA, about 10 μM DTPA, about 15 μM DTPA, and/or about 20 μM DTPA, preferably wherein the formulation comprises about 10 μM DTPA.

9. The liquid formulation of any one of claims 1 to 8, wherein the formulation has a pH of about 7.0 to about 7.8, about 7.2 to about 7.8, about 7.4 to about 7.6, about 7.0 to about 7.2, about 7.2 to about 7.4, about 7.4 to about 7.6, about 7.6 to about 7.8, about 7.8 to about 8.0, about 6.8, about 6.9, about 7.0, about 7.1, about 7.2, about 7.3, about 7.4, about 7.5, about 7.6, about 7.7, about 7.8, about 7.9, and/or about 8.0, preferably wherein the formulation has a pH of about 7.5.

10. A liquid pharmaceutical formulation, comprising:
about 20 μg/mL fusion protein of SEQ ID NO: 11;
about 50 mM alanine;
about 200 mM trehalose;
about 25 mM glycylglycine;
about 0.02% polysorbate 20 (v/v); and
about 10 μM DTPA,
wherein the formulation has a pH of about 7.5.

11. The liquid formulation of any one of claims 1 to 10, wherein the formulation is a reconstituted formulation.

12. The liquid formulation of claim 11, wherein the reconstituted formulation is reconstituted from a lyophilized formulation.

13. A solid pharmaceutical formulation, comprising:
about 0.01% to about 0.03% (w/w) fusion protein of SEQ ID NO: 11;
about 2% to about 8% alanine (w/w);
about 50% to about 95% trehalose (w/w);
about 2% to about 8% glycylglycine (w/w);
about 0.1% to about 0.4% polysorbate 20 (w/w); and
about 0.002% to about 0.006% diethylenetriaminepentaacetic acid (DTPA) (w/w).

14. The solid pharmaceutical formulation of claim 13, comprising:
about 0.024 to about 0.028% (w/w) fusion protein of SEQ ID NO: 11;
about 4.0% and about 5.0% alanine (w/w);
about 70% to about 80% trehalose (w/w);
about 3.0% to about 4.0% glycylglycine (w/w);
about 0.15% to about 0.30% polysorbate 20 (w/w); and
about 0.0045% to about 0.0055% DTPA (w/w).

15. The formulation of claim 13 or 14, wherein the formulation is a lyophilized formulation.

16. The formulation of any one of the preceding claims, wherein the formulation is suitable for parenteral administration.

17. The formulation of any one of claims 1 to 15, wherein the formulation is suitable for intravenous administration.

18. The formulation of any one of claims 1 to 15, wherein the formulation is suitable for subcutaneous administration.

19. A method of treating an IL-12-related disease or disorder in a subject in need thereof, the method comprising administering the formulation of any one of claims 1 to 15 to the subject.
